(19) United States
(12) Patent Application Publication
(10) Pub. No.: US 2024/0207377 A1
(43) Pub. Date: Jun. 27, 2024

(54) ALPHA-1-ANTITRYPSIN (AAT) IN THE TREATMENT AND/OR PREVENTION OF NEUROLOGICAL DISORDERS

(71) Applicant: AGERONIX SA, Carouge (CH)

(72) Inventor: Nikolay ZHUKOVSKY, Chambesy (CH)

(21) Appl. No.: 18/288,654

(22) PCT Filed: May 3, 2022
(86) PCT No.: PCT/EP2022/061882

§ 371 Date: Oct. 27, 2023

(30) Foreign Application Priority Data
May 3, 2021 (WO) PCT/EP2021/061597
Nov. 2, 2021 (EP) 21206096.6
Apr. 14, 2022 (EP) 22168622.3

(51) Int. Cl. A61K 38/55; A61P 25/28

(52) U.S. Cl. CPC A61K 38/55; A61P 25/28

(57) ABSTRACT

The invention relates to a composition comprising a therapeutically effective amount of an alpha1-antitrypsin (AAT) protein, a variant, an isoform and/or a fragment thereof or a vector or a genetically modified cell comprising a sequence encoding AAT for use in the treatment and/or prevention of a disease or disorder of the nervous system or a symptom thereof.

The invention relates to a composition comprising a therapeutically effective amount of an alpha1-antitrypsin (AAT) protein, a variant, an isoform and/or a fragment thereof or a vector or a genetically modified cell comprising a sequence encoding AAT for use in the treatment and/or prevention of a disease or disorder of the nervous system or a symptom thereof.

Diseases or disorders of the nervous system are diseases or disorders which can dramatically affect the peripheral and/or central nervous system (PNS/CNS). In the last decade, neuroinflammation has become more and more central in our understanding of neurological disorders. Inflammation per se may directly or indirectly trigger the disease but it does undoubtedly contribute to the pathogenesis of the disease throughout the peripheral (PNS) and central nervous systems (CNS). Peripheral diseases like Guillain-Barré Syndrome (GBS) (Chang et al. 2012), Charcot-Marie-Tooth Disease (Hoyle et al. 2015), neuropathic pain, fibromyalgia and other neuropathies (PN) (Martin-Aguilar, Pascual-Goni, and Querol 2019) as well as central diseases including Parkinson's (PD) and motor neuron disease (Marogianni et al. 2020), Alzheimer's diseases (AD) (Hampel et al. 2020) and other dementias, Multiple Sclerosis (MS) (Baecher-Allan, Kaskow, and Weiner 2018; Matthews 2019), Amyotrophic Lateral Sclerosis (ALS), ischemia and traumatic brain injuries, depression and autism spectrum disorder have been all linked to mechanisms driven by activated microglia (Skaper et al. 2018).

Hereditary, peripheral neuropathies constitute a highly diverse group of disorders whose most frequent form is collectively known as Charcot-Marie-Tooth disease (CMT), with a world-wide prevalence of 1:2,500 and a high genetic heterogeneity (>100 different genes involved) (Bird, T. D., 1993, GeneReviews®). In this broad range of genetical pattern possibilities, CMT1A is the most common form and accounts for 80% of the CMTs of type 1. It is characterized by intra-chromosomal duplication of the PMP22 gene (Stavrou, Sargiannidou et al. 2021). The major component of peripheral neuropathies is damages to the myelin sheath, either after its abnormal development (dysmyelination) in the inherited forms (CMT1A-F and -X) or direct in the acquired ones (acute/chronic inflammatory demyelinating polyneuropathy; AIDP/CIDP).

Myelin is produced by Schwann cells (SCs) in the PNS and is crucial for proper transmission of the electric impulse in the nerves. In the intricate neuron/glia cross-communication that is required for proper myelin regulation (Rao and Pearse 2016), several diverse signaling pathways are involved, which include growth factors, integrins and cell adhesion molecules but more importantly, the pivotal neuregulin 1 type III (NRG1-III) signal through ERBB2/3 receptors (Taveggia, C., et al., 2005, Neuron 47(5): 681-694.) and its proteolytic sheddase modulator, the tumor necrosis factor-α-converting enzyme, TACE (also known as ADAM17) (Fleck, D. et al., 2016, J Biol Chem 291(1): 318-333). Although diverse and cutting-edge therapeutic strategies are currently being explored (CRISPR/Cas9 editing, viral-based gene delivery, siRNA nanoparticle), none successfully completed a phase III in clinical trials and CMTs are left with no actual treatments (Fridman, V. and M. A. Saporta, 2021, Neurotherapeutics 18(4): 2236-2268.). Moreover, very few of these strategies target the NRG1/EBRB2/3/TACE pathway even though inhibition of TACE has been shown to promote myelination. TACE/ADAM17, is a transmembrane protein that includes an extracellular zinc-dependent protease domain. In the context of CMT1A, ADAM17 is known for its inhibitory effect on SCs mediated myelination by cleaving NRG1-III in the epidermal growth factor domain in a ligand independent manner (La Marca, R., 2011, Nat Neurosci 14(7): 857-865.). Conflicting evidence has been reported in the literature with respect to the role of the human protease alpha-1-Antitrypsin (AAT), specifically, in 2013 AAT was shown not to interact with TACE (van′t Wout E. F. et al., 2014, Hum Mol Genet.; 23(4):929-4) in contrast to an earlier report in 2010 that claimed AAT does indeed interact with TACE and inhibits its activity in a dose dependent manner (Bergin, D. A. et al., 2010, J Clin Invest 120(12): 4236-4250.).

The variety of impairments caused by neurological disorders has been increasingly considered as a worldwide public health challenge and its burden is expected to rise in the coming decades.

Diseases or disorders of the nervous system can be caused by viruses. Viruses, such as for example Coronaviruses, cause diseases, including diseases or disorders of the nervous system, in animals and humans around the world. Coronaviruses are RNA viruses.

Human coronaviruses (HCoV) are mainly known to cause infections of the upper and lower respiratory tract. Examples for human coronaviruses are: a beta coronavirus that causes Middle East Respiratory Syndrome (named MERS-CoV), a beta coronavirus that causes severe acute respiratory syndrome (named SARS-CoV, or SARS-CoV-1), a novel coronavirus that causes coronavirus disease 2019 or COVID-19 (named SARS-CoV-2), alpha coronavirus 299E, alpha coronavirus NL63, beta coronavirus OC43, and beta coronavirus HKU1.

The disease or syndrome caused by a SARS-CoV-2 infection is also referred to as COVID-19. A SARS-CoV-2 infection can be either asymptomatic or lead to a disease or syndrome related to mild or severe symptoms. The most common symptoms of a disease or syndrome related to a SARS-CoV-2 (also referred to as SARS-CoV-19) infection are fever and cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste. Further symptoms include lack of appetite, loss of weight, stomach pain, conjunctivitis, skin rash, lymphoma, apathy, and somnolence.

Patients with severe symptoms can develop pneumonia. A significant number of patients with pneumonia require passive oxygen therapy. Non-invasive ventilation and high-flow nasal oxygen therapy can be applied in mild and moderate non-hypercapnia pneumonia cases. A lung-saving ventilation strategy must be implemented in severe acute respiratory syndrome or acute respiratory distress syndrome (SARS/ARDS) and mechanically ventilated patients.

While the principal complication of coronavirus disease 2019 (COVID-19) is respiratory failure, a considerable number of patients have been reported with neurological symptoms affecting both the peripheral and central nervous systems (Niazkar, Zibaee et al. 2020 Neurol Sci 41(7): 1667-1671; Nordvig, Fong et al. 2021, Neurol Clin Pract 11(2): e135-e146). The hematogenic pathway, retro-/antero-grade transport along peripheral nerves as well as rare direct invasion, are considered as potential neuroinvasion mechanisms of neurotropic virus including SARS-CoV-2 (Barrantes 2021 Brain Behav Immun Health 14: 100251; Tavcar, Potokar et al. 2021, Front Cell Neurosci 15: 662578). Severe cases of SARS-CoV-2 often exhibit disproportionate and abnormal inflammatory responses, including systemic upregulation of cytokines, chemokines, and pro-inflammatory cues (Najjar et al. 2020, J Neuroinflammation 17(1): 231). Such systemic hyper-inflammation could impair the neurovascular endothelial function, damage the blood brain barrier ultimately activating CNS immune system and contributing to CNS complications (Amruta, Chastain et al., 2021, Cytokine Growth Factor Rev 58: 1-15).

Despite the attention that the COVID-19 pandemic has recently taken, several other viruses are associated with major brain disorders like Alzheimer's, Parkinson's and multiple sclerosis' disease. The diseases or syndromes related to virus infections further include a wide range of diseases or syndromes such as inflammatory diseases and are a major burden for society.

The biological common trait of many CNS and PNS neurodegenerative diseases is a sustained and acute inflammatory response due to cytokine release orchestrated in feed-forward loops (also called “cytokine storm”). Therefore dampening of the inflammatory reaction stands as a central target of therapeutical strategies. However, the subtleties of inflammatory mechanisms underlying its multiple mediators are not fully understood.

Thus, there is a need for improved therapies for diseases or disorders of the nervous system.

The above technical problem is solved by the embodiments disclosed herein and as defined in the claims.

Accordingly, the invention relates to, inter alia, the following embodiments:
1. A composition for use in the treatment and/or prevention of a disease or disorder of the nervous system, the composition comprising a therapeutically effective amount of an alpha1-antitrypsin (AAT) protein, a variant, an isoform and/or a fragment thereof.
2. A vector comprising a nucleic acid sequence encoding an AAT protein for use in the treatment and/or prevention of a disease or disorder of the nervous system.
3. A genetically modified cell comprising a nucleic acid sequence encoding an AAT protein for use in the treatment and/or prevention of a disease or disorder of the nervous system.
4. The composition for use of embodiment 1, the vector for use of embodiment 2 or the genetically modified cell for use of embodiment 3, wherein the disease or disorder of the nervous system is an inflammatory disease or disorder of the nervous system.
5. The composition for use of embodiment 4, the vector for use of embodiment 4 or the genetically modified cell for use of embodiment 4, wherein the inflammatory disease or disorder of the nervous system is a myeloid cell-mediated disease or disorder of the nervous system.
6. The composition for use of any one of embodiments 1, 4 or 5, the vector for use of any one of embodiments 2, 4 or 5 or the genetically modified cell for use of any one of embodiments 3 to 5, wherein the disease or syndrome of the nervous system is a disease or syndrome selected from the group of dementia, multiple sclerosis, amyotrophic lateral sclerosis, Alzheimer's disease, Parkinson's disease and Huntington's disease.
7. The composition for use of any one of embodiments 1, 4 to 6, the vector for use of any one of embodiments 2, 4 to 6 or the genetically modified cell for use of any one of embodiments 3 to 6, wherein the disease or disorder of the nervous system is at least one symptom of a disease or disorder of the nervous system selected from the group consisting of: tremor, memory loss, slurred speech, dizziness, change in vision and headache.
8. The composition for use of embodiment 1, the vector for use of embodiment 2 or the genetically modified cell for use of embodiment 3, wherein the disease or disorder of the nervous system is a disease or disorder of the peripheral nervous system.
9. The composition for use of embodiment 8, the vector for use of embodiment 8 or the genetically modified cell for use of embodiment 8, wherein the disease or disorder of the peripheral nervous system is motor and sensory neuropathy of the peripheral nervous system.
10. The composition for use of embodiment 9, the vector for use of embodiment 9 or the genetically modified cell for use of embodiment 9, wherein the sensory neuropathy of the peripheral nervous system is a hereditary motor and sensory neuropathy of the peripheral nervous system.
11. The composition for use of embodiment 10, the vector for use of embodiment 10 or the genetically modified cell for use of embodiment 10, wherein the hereditary motor and sensory neuropathy of the peripheral nervous system is Charcot-Marie-Tooth disease or a symptom thereof, preferably at least one symptom selected from the group consisting of weakness in legs, ankles and/or feet, loss of muscle bulk in legs and/or feet, high foot arches, curled toes, decreased ability to run, difficulty lifting foot at the ankle, abnormal gait, frequent tripping or falling and decreased sensation or a loss of feeling in legs and/or feet.
12. The composition for use of any one of embodiments 1, 4 to 11, wherein the AAT protein, a variant, an isoform and/or a fragment thereof is human plasma-extracted.
13. The composition for use of any one of embodiments 1, 4 to 11, wherein the alpha1-antitrypsin (AAT) protein, a variant, an isoform and/or a fragment thereof is recombinant alpha1-antitrypsin (rhAAT), a variant, an isoform and/or a fragment thereof.
14. The composition for use of any one of embodiments 1, 4 to 13, wherein the composition comprises at least one pharmaceutical carrier.
15. The composition for use of embodiment 14, wherein the pharmaceutical carrier is a blood-brain barrier permeability enhancer.
16. The composition for use of any one of embodiments 1, 4 to 11, the vector for use of any one of embodiments 2, 4 to 6 or the genetically modified cell for use of any one of embodiments 3 to 7, wherein the composition, the vector or the genetically modified cell is formulated for intracerebral administration, intravenous injection, intravenous infusion, infusion with a dosator pump, inhalation nasal-spray, eye-drops, skin-patches, slow release formulations, ex vivo gene therapy or ex vivo cell-therapy.

Accordingly, in one embodiment, the invention relates to a composition for use in the treatment and/or prevention of a disease or disorder of the nervous system, the composition comprising a therapeutically effective amount of an alpha1-antitrypsin (AAT) protein, a variant, an isoform and/or a fragment thereof.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “disease or disorder of the nervous system”, as used herein, refers to a group of disease or disorders, wherein the pathology involves the nervous system. In some embodiments, the disease or disorder of the nervous system described herein is a disease or disorder selected from the group consisting of 12q14 microdeletion syndrome, 15q13.3 microdeletion syndrome, 15q24 microdeletion syndrome, 22q11.2 deletion syndrome, 22q13.3 deletion syndrome, 2-methylbutyryl-CoA dehydrogenase deficiency, 2q23.1 microdeletion syndrome, 2q37 deletion syndrome, 3-alpha hydroxyacyl-CoA dehydrogenase deficiency, 3MC syndrome, XXXY syndrome, XYYY syndrome, XXXXY syndrome, 5q14.3 microdeletion syndrome, 6-pyruvoyl-tetrahydropterin synthase deficiency, Aarskog syndrome, Abetalipoproteinemia, ABri amyloidosis, absence of septum pellucidum, Aceruloplasminemia, Acrocallosal syndrome, acrofacial dysostosis Catania type, acrofacial dysostosis Rodriguez type, acute cholinergic dysautonomia, acute CNS demyelinating event, acute disseminated encephalomyelitis, acute intermittent porphyria, acute motor and sensory axonal neuropathy syndrome, ADCY5-related dyskinesia, adenosine monophosphate deaminase 1 deficiency, adenylosuccinase deficiency, Adie syndrome, adrenomyeloneuropathy, adult polyglucosan body disease, adult-onset nemaline myopathy, advanced sleep phase syndrome, agenesis of the corpus callosum, age-related peripheral neuropathy, age-related peripheral neuropathy, Agnosia, Aicardi syndrome, Aicardi-Goutieres syndrome, AIDS Dementia Complex, Al Gazali Aziz Salem syndrome, Alaninuria, Albinism deafness syndrome, alcohol or nutritional deficiencies induced sensorimotor deficiency, alcoholic neuropathy, alcoholic peripheral neuropathy, Alexander disease, ALG11-CDG (CDG-Ip), ALG12-CDG (CDG-Ig), ALG13-CDG, ALG1-CDG (CDG-Ik), ALG2-CDG (CDG-Ii), ALG3-CDG (CDG-Id), ALG6-CDG (CDG-Ic), ALG8-CDG (CDG-Ih), ALG9-CDG (CDG-IL), Allan-Herndon-Dudley syndrome, alopecia epilepsy oligophrenia syndrome of Moynahan, alopecia, epilepsy, pyorrhea, mental subnormality, alopecia-contractures-dwarfism-intellectual disability syndrome, alopecia-intellectual disability syndrome, Alpers syndrome, alpha-ketoglutarate dehydrogenase deficiency, alpha-mannosidosis, alpha-thalassemia x-linked intellectual disability syndrome, alternating hemiplegia of childhood, Alzheimer disease type 4, Alzheimer's disease, Alzheimer's disease without neurofibrillary tangles, aminoacylase 1 deficiency, aminolevulinate dehydratase deficiency porphyria, Amish lethal microcephaly, Amish Nemaline Myopathy, amyloid neuropathy, amyopathic dermatomyositis, amyotrophic lateral sclerosis, amyotrophic lateral sclerosis type 6, amyotrophic lateral sclerosis-parkinsonism/dementia complex 1, amytrophic lateral sclerosis, anaplastic astrocytoma, anaplastic ganglioglioma, anaplastic oligodendroglioma, Andermann syndrome, Andersen-Tawil syndrome, anemia sideroblastic ataxia, spinocerebellar ataxia, Anencephaly, Angioma hereditary neurocutaneous, Aniridia, Aniridia renal agenesis psychomotor retardation, Antisynthetase syndrome, Aortic arch anomaly, Apraxia, Arachnoid cysts, Arachnoiditis, Aromatic L-amino acid decarboxylase deficiency, Arthrogryposis multiplex congenita, distal, X-linked, Arthrogryposis renal dysfunction cholestasis syndrome, Arts syndrome, Aspartylglycosaminuria, Ataxia, Ataxia telangiectasia, Ataxia with oculomotor apraxia type 1, Ataxia with Oculomotor Apraxia Type 2, Ataxia with oculomotor apraxia type 4, Ataxia with vitamin E deficiency, Ataxia-teleangiectasia, Atelosteogenesis type 2, Atelosteogenesis type 3, Atkin syndrome, Atypical Rett syndrome, Autism with port-wine stain, Autosomal dominant centronuclear myopathy, Autosomal dominant cerebellar ataxia/deafness/narcolepsy, autosomal dominant Charcot-Marie-Tooth disease type 2 with giant axons, autosomal dominant deafness-onychodystrophy syndrome, autosomal dominant intermediate Charcot-Marie-Tooth disease, autosomal dominant leukodystrophy with autonomic disease, autosomal dominant neuronal ceroid lipofuscinosis 4B, autosomal dominant nocturnal frontal lobe epilepsy, autosomal dominant non-syndromic intellectual disability, autosomal dominant optic atrophy plus syndrome, autosomal dominant partial epilepsy with auditory features, autosomal dominant spinal muscular atrophy, autosomal recessive axonal neuropathy with neuromyotonia, autosomal recessive centronuclear myopathy, autosomal recessive Charcot-Marie-Tooth disease with hoarseness, autosomal recessive intermediate Charcot-Marie-Tooth disease type A, autosomal recessive intermediate Charcot-Marie-Tooth disease type B, autosomal recessive juvenile Parkinson disease, Autosomal recessive neuronal ceroid lipofuscinosis 4A, Adult neuronal ceroid lipofuscinosis, Autosomal recessive primary microcephaly, Autosomal recessive spastic ataxia 4, Autosomal recessive spastic paraplegia type 49, Autosomal recessive spinocerebellar ataxia 9, B4GALT1-CDG (CDG-IId), Bannayan-Riley-Ruvalcaba syndrome, Barth syndrome, Battaglia-Neri syndrome, Becker muscular dystrophy, Behavioral variant of frontotemporal dementia, Behçet disease, Bell's palsy, Benign essential blepharospasm, Benign familial neonatal epilepsy, Benign familial neonatal-infantile seizures, Benign hereditary chorea, Benign rolandic epilepsy (BRE), Beta-Propeller Protein-Associated Neurodegeneration, Bethlem myopathy, bilateral frontal polymicrogyria, bilateral frontoparietal polymicrogyria, bilateral generalized polymicrogyria, bilateral parasagittal parieto-occipital polymicrogyria, bilateral perisylvian polymicrogyria, Binswanger's disease, Biotinidase deficiency, Biotin-thiamine-responsive basal ganglia disease, Birk-Barel syndrome, Bixler Christian Gorlin syndrome, Blepharonasofacial malformation syndrome, Bobble-head doll syndrome, Bohring-Opitz syndrome, Borjeson-Forssman-Lehmann syndrome, Bowen-Conradi syndrome, Brachioskeletogenital syndrome, Brachydactyly-mesomelia-intellectual disability-heart defects syndrome, Brain dopamine-serotonin vesicular transport disease, Brain-lung-thyroid syndrome, Branchial arch syndrome X-linked, Brody myopathy, Brooks Wisniewski Brown syndrome, Brown-Sequard syndrome, Bullous dystrophy, C syndrome, Cabezas syndrome, CADASIL, Camptocormism, Camptodactyly arthropathy coxa vara pericarditis syndrome, CANOMAD syndrome, Cantu syndrome, Cap myopathy, Cardiofaciocutaneous syndrome, Carey-Fineman-Ziter syndrome, Carney complex, Cataract ataxia deafness, Catel Manzke syndrome, Caudal appendage deafness, Caudal regression sequence, Central core disease, Central nervous system germinoma, Central neurocytoma, Central pain syndrome, Central pontine myelinolysis, Cerebellar ataxia, Cerebellar degeneration, Cerebellar hypoplasia, Cerebelloparenchymal disorder 3, Cerebellum agenesis hydrocephaly, Cerebral autosomal recessive arteriopathy, Cerebral cavernous malformation, Cerebral dysgenesis, neuropathy, ichthyosis, and palmoplantar keratoderma syndrome, Cerebral folate deficiency, Cerebral gigantism jaw cysts, Cerebral palsy, Cerebral palsy ataxic, Cerebral palsy athetoid, Cerebral palsy spastic hemiplegic, Cerebral palsy spastic monoplegic, Cerebral palsy spastic quadriplegic, Cerebral sclerosis, Cerebro-facio-articular syndrome, Cerebro-oculo-facio-skeletal syndrome, Cerebrooculonasal syndrome, Cerebrospinal fluid leak, Cerebrotendinous xanthomatosis, Ceroid lipofuscinosis neuronal 1, Cervical hypertrichosis peripheral neuropathy, Chanarin-Dorfman syndrome, Charcot-Marie-Tooth disease, Charcot-Marie-Tooth disease type 1A, Chediak-Higashi syndrome, Chiari malformation, Chiari malformation type 1, Chiari malformation type 2, Chiari malformation type 4, Childhood apraxia of speech, Childhood-onset nemaline myopathy, Chorea-acanthocytosis, Choroid plexus carcinoma, Choroid plexus papilloma, Christianson syndrome, Chromosome 17p13.1 deletion syndrome, Chromosome 17q11.2 deletion syndrome, Chromosome 19q13.11 deletion syndrome, Chromosome 1p36 deletion syndrome, Chromosome 3p-syndrome, Chronic hiccups, Chronic lymphocytic inflammation, Chronic progressive external ophthalmoplegia, Chudley Rozdilsky syndrome, Cisplatin induced sensory neuropathy, Cleft palate short stature vertebral anomalies, Cluster headache, COACH syndrome, COASY protein-associated neurodegeneration, coats disease, Cobb syndrome, Cockayne syndrome type I, Cockayne syndrome type II, Cockayne syndrome type III, Coenzyme Q10 deficiency, Coffin-Lowry syndrome, Coffin-Siris syndrome, COG1-CDG (CDG-IIg), COG4-CDG (CDG-IIj), COG5-CDG (CDG-IIi), COG7-CDG (CDG-IIe), COG8-CDG (CDG-IIh), Cohen syndrome, cold-induced sweating syndrome, complex regional pain syndrome, congenital central hypoventilation syndrome, congenital cytomegalovirus, congenital fiber type disproportion, congenital fibrosis of extraocular muscles, congenital generalized lipodystrophy type 4, congenital insensitivity to pain, congenital insensitivity to pain with anhidrosis, congenital intrauterine infection-like syndrome, congenital laryngeal palsy, congenital mirror movement disorder, congenital muscular dystrophy, congenital myasthenic syndrome, congenital rubella, congenital toxoplasmosis, continuous spike-wave during slow sleep syndrome, convulsions, corneal hypesthesia, Cornelia de Lange syndrome, Corpus callosum agenesis, Cortical blindness, Cortical dysgenesis, Corticobasal degeneration, Costello syndrome, Crane-Heise syndrome, Craniofrontonasal dysplasia, Craniopharyngioma, Craniorachischisis, Craniotelencephalic dysplasia, Creutzfeldt-Jakob disease, Crome syndrome, Curry Jones syndrome, cylindrical spirals myopathy, cyprus facial neuromusculoskeletal syndrome, cytomegalic inclusion disease, D-2-hydroxyglutaric aciduria, Dandy-Walker cyst, Dandy-Walker like malformation, Dandy-Walker malformation, Danon disease, Dapsone induced neuropathy, DDOST-CDG (CDG-Ir), DEAF1-associated disorders, Dentatorubral-pallidoluysian atrophy, Dermatomyositis, Developmental dysphasia familial, Diabetic neuropathy, Dihydrolipoamide dehydrogenase deficiency, Dihydropteridine reductase deficiency, Diphtheria, Distal myopathy with vocal cord weakness, DOOR syndrome, Dopamine beta hydroxylase deficiency, Dopamine transporter deficiency syndrome, Dopa-responsive dystonia, DPAGT1-CDG (CDG-Ij), DPM1-CDG (CDG-Ie), DPM2-CDG, DPM3-CDG (CDG-Io), Dravet syndrome, Duane syndrome, Dubowitz syndrome, Duchenne muscular dystrophy, Dykes Markes Harper syndrome, Dysautonomia like disorder, Dysequilibrium syndrome, Dyskeratosis congenita, Dyskeratosis congenita autosomal dominant, Dyskeratosis congenita autosomal recessive, Dyskeratosis congenita X-linked, Dyssynergia cerebellaris myoclonica, Dystonia 2, DYT-PRKRA, DYT-THAP1, DYT-TOR1A, DYT-TUBB4A, early infantile epileptic encephalopathy, early infantile epileptic encephalopathy 25, early-onset anterior polar cataract, early-onset autosomal dominant alzheimer disease, early-onset parkinsonism-intellectual disability syndrome, eastern equine encephalitis, empty sella syndrome, encephalitis lethargica, encephalocraniocutaneous lipomatosis, encephalopathy, eosinophilic fasciitis, eosinophilic granulomatosis, ependymoma, epidermolysa bullosa simplex with muscular dystrophy, epilepsy juvenile absence, epilepsy occipital calcifications, epilepsy progressive myoclonic type 3, epilepsy with myoclonic-atonic seizures, epiphyseal dysplasia hearing loss dysmorphism, episodic ataxia, erythromelalgia, essential tremor, Fabry disease, facial onset neuronopathy, facioscapulohumeral muscular dystrophy, Fallot complex, familial amyloidosis, familial bilateral striatal necrosis, familial caudal dysgenesis, familial congenital palsy of trochlear nerve, familial dysautonomia, familial encephalopathy, familial exudative vitreoretinopathy, familial focal epilepsy, familial hemiplegic migraine, familial hemophagocytic lymphohistiocytosis, familial infantile convulsions familial infantile paroxysmal choreoathetosis, familial porencephaly, familial transthyretin amyloidosis, familiar or sporadic hemiplegic migraine, farber disease, fatal familial insomnia, fatal infantile encephalomyopathy, fatty acid hydroxylase-associated neurodegeneration, FBXL4-related encephalomyopathic mitochondrial DNA depletion syndrome, Febrile infection-related epilepsy syndrome, Feigenbaum Bergeron Richardson syndrome, Filippi syndrome, Fine-Lubinsky syndrome, Fingerprint body myopathy, Fitzsimmons Walson Mellor syndrome, Fitzsimmons-Guilbert syndrome, Floating-Harbor syndrome, Flynn Aird syndrome, focal dermal hypoplasia, focal segmental glomerulosclerosis, Fountain syndrome, FOXG1 syndrome, Fragile X syndrome, Fragile XE syndrome, Friedreich ataxia, Frontometaphyseal dysplasia, Frontotemporal dementia, Frontotemporal lobar dementia, Fryns syndrome, Fucosidosis, Fukuyama type muscular dystrophy, Fumarase deficiency, Galactosialidosis, Galloway-Mowat syndrome, Gamma aminobutyric acid transaminase deficiency, Gangliocytoma, GAPO syndrome, Gaucher disease type 1, Gaucher disease type 2, Gaucher disease type 3, Gemignani syndrome, Genitopatellar syndrome, Genoa syndrome, Gerstmann syndrome, Gerstmann-Straussler-Scheinker disease, Giant axonal neuropathy, Gillespie syndrome, Gliomatosis cerebri, Glucose transporter type 1 deficiency syndrome, Glutamine deficiency, congenital, Glutaric acidemia type 1, Glutaric acidemia type II, Glutaric acidemia type III, Glycogen storage disease type 13, Glycogen storage disease type 2, Glycogen storage disease type 3, Glycogen storage disease type 4, Glycogen storage disease type 5, Glycogen storage disease type 7, GM1 gangliosidosis type 1, GM1 gangliosidosis type 2, GM1 gangliosidosis type 3, GM3 synthase deficiency, GMS syndrome, Goldberg-Shprintzen megacolon syndrome, Gomez Lopez Hernandez syndrome, GOSR2-related progressive myoclonus ataxia, Graham-Cox syndrome, Granulomatosis with polyangiitis, Griscelli syndrome type 1, Grubben de Cock Borghgraef syndrome, GTP cyclohydrolase I deficiency, GTPCH1-deficient DRD, Guanidinoacetate methyltransferase deficiency, Guillain-Barre syndrome, Gurrieri syndrome, Gyrate atrophy of choroid and retina, Hair defect-photosensitivity-intellectual disability syndrome, Hallermann-Streiff syndrome, Hall-Riggs syndrome, Hamanishi Ueba Tsuji syndrome, Hansen's disease, Harding ataxia, Harlequin syndrome, Harrod Doman Keele syndrome, Hartnup disease, Hashimoto encephalopathy, Hemangioblastoma, Hemicrania continua, Hemimegalencephaly, Hennekam syndrome, hereditary angiopathy, hereditary coproporphyria, hereditary diffuse leukoencephalopathy, hereditary fibrosing poikiloderma with tendon contractures, myopathy, and pulmonary fibrosis, hereditary geniospasm, hereditary hemorrhagic telangiectasia, hereditary hemorrhagic telangiectasia type 2, hereditary hemorrhagic telangiectasia type 3, hereditary hemorrhagic telangiectasia type 4, hereditary hyperekplexia, hereditary motor and sensory neuropathy type 5, hereditary neuropathy with liability to pressure palsies, hereditary predisposition to pressure palsies (focal and symmetrical), hereditary proximal myopathy with early respiratory failure, hereditary sensorimotor neuropathy with hyperelastic skin, hereditary sensory and autonomic neuropathy type 1e, hereditary sensory and autonomic neuropathy type 2, hereditary sensory and autonomic neuropathy type 7, hereditary sensory and autonomic neuropathy type v, hereditary sensory neuropathy type 1, hereditary spastic paraplegia, hereditary vascular retinopathy, Hernendez-Aguirre Negrete syndrome, herpes simplex encephalitis, herpes zoster oticus, HIBCH deficiency, Homocystinuria, Horizontal gaze palsy with progressive scoliosis, Hoyeraal Hreidarsson syndrome, HSD10 disease, HTLV-1 associated myelopathy/tropical spastic paraparesis, Human HOXA1 Syndromes, Human immunodeficiency virus induced neuropathy, Huntington disease, Huntington's disease, Hurler syndrome, Hurler-Scheie syndrome, hydranencephaly, hydrocephalus (e.g. due to congenital stenosis of aqueduct of sylvius), hydrocephalus-cleft palate-joint contractures syndrome, hydroxykynureninuria, hyperbetaalaninemia, hypercoagulability syndrome due to glycosylphosphatidylinositol deficiency, hyperkalemic periodic paralysis, hypermethioninemia, hyperphenylalaninemia, hyperprolinemia, hyperprolinemia type 2, hypertrophic neuropathy of Dejerine-Sottas, hypocalcemia, autosomal dominant, hypokalemic periodic paralysis, hypomelanosis of Ito, hypomyelination (e.g., with atrophy of basal ganglia and/or cerebellum), hypoparathyroidism-intellectual disability-dysmorphism syndrome, hypospadias-intellectual disability, Goldblatt type syndrome, hypothalamic hamartomas, ichthyosis alopecia eclabion ectropion intellectual disability, idiopathic intracranial hypertension, idiopathic spinal cord herniation, inclusion body myositis, incontinentia pigmenti, infantile axonal neuropathy, infantile cerebellar retinal degeneration, infantile choroidocerebral calcification syndrome, infantile myofibromatosis, infantile neuroaxonal dystrophy, infantile onset spinocerebellar ataxia, infantile spasms broad thumbs, infantile-onset ascending hereditary spastic paralysis, infection-induced acute encephalopathy 3, intellectual deficit Buenos-Aires type, athetosis intellectual disability, hypoplastic corpus callosum intellectual disability, intellectual disability-developmental delay-contractures syndrome, intellectual disability-dysmorphism-hypogonadism-diabetes mellitus syndrome, Intellectual disability-severe speech delay-mild dysmorphism syndrome, intellectual disability-spasticity-ectrodactyly syndrome, Intermediate congenital nemaline myopathy, Internal carotid agenesis, Intraneural perineurioma, IRVAN syndrome, Isaacs' syndrome, Isodicentric chromosome 15 syndrome, Johanson-Blizzard syndrome, Johnson neuroectodermal syndrome, Joubert syndrome, Juberg Marsidi syndrome, Juvenile amyotrophic lateral sclerosis, Juvenile dermatomyositis, Juvenile Huntington disease, Juvenile polymyositis, Juvenile primary lateral sclerosis, Kabuki syndrome, Kanzaki disease, Kapur Toriello syndrome, Kaufman oculocerebrofacial syndrome, KBG syndrome, KCNQ2-Related Disorders, Kearns-Sayre syndrome, Kennedy disease, Keratosis follicularis dwarfism, cerebral atrophy, Kernicterus, Keutel syndrome, King Denborough syndrome, Kleine Levin syndrome, Klumpke paralysis, Kosztolanyi syndrome, Kozlowski-Krajewska syndrome, Krabbe's disease, Kuru, Kuzniecky Andermann syndrome, L-2-hydroxyglutaric aciduria, La Crosse encephalitis, Laband syndrome, Lafora disease, Laing distal myopathy, Lambert Eaton myasthenic syndrome, Landau-Kleffner syndrome, I-arginine:glycine amidinotransferase deficiency, Late-onset distal myopathy, Markesbery-Griggs type, lateral meningocele syndrome, Laurence-Moon syndrome, LCHAD deficiency, Leber hereditary optic neuropathy, Leigh syndrome, Lennox-Gastaut syndrome, Lenz Majewski hyperostotic dwarfism, Lenz microphthalmia syndrome, Lesch Nyhan syndrome, Leukodystrophy, Leukoencephalopathy (e.g. with thalamus and brainstem involvement and high lactate), Levic Stefanovic Nikolic syndrome, Lewis-Sumner syndrome, Lhermitte-Duclos disease, Li-Fraumeni syndrome, limb-girdle muscular dystrophy (e.g. type 1A, 1B, 1C, 1D, 1E, 1F, 1G, 1H, 2A, 2B, 2C, 2D, 2E, 2F, 2H, 2I, 2J, 2K, 2L, 2M, 2N, 2O, 2P, 2Q, 2S, 2T), limbic encephalitis with LGI1 antibodies, Limited cutaneous systemic sclerosis, Lipoic acid synthetase deficiency, Lissencephaly 1, Lissencephaly 2, Lissencephaly X-linked, Localized hypertrophic neuropathy, Locked-in syndrome, Logopenic progressive aphasia, Lowe oculocerebrorenal syndrome, Lowry Maclean syndrome, Lujan syndrome, Lyme disease, Mac Dermot Winter syndrome, macrocephaly-short stature-paraplegia syndrome, macrothrombocytopenia progressive deafness, mal de debarquement syndrome, male pseudohermaphroditism intellectual disability syndrome, malignant hyperthermia, malignant hyperthermia arthrogryposis torticollis, malignant migrating partial seizures of infancy, MAN1B1-CDG, Mandibulofacial dysostosis (e.g. with microcephaly), Mannosidosis, Marchiafava Bignami disease, Marden-Walker syndrome, Marfanoid habitus-autosomal recessive intellectual disability syndrome, Marinesco-Sjogren syndrome, Martsolf syndrome, McDonough syndrome, McLeod neuroacanthocytosis syndrome, Meckel syndrome, MECP2 duplication syndrome, medrano roldan syndrome, medulloblastoma, megalencephalic leukoencephalopathy (e.g. with subcortical cysts), megalencephaly-polymicrogyria-polydactyly-hydrocephalus syndrome, megaloblastic anemia, megalocornea-intellectual disability syndrome, Mehes syndrome, MEHMO syndrome, Meier-Gorlin syndrome, Meige syndrome, Melnick-Needles syndrome, Meningioma, meningitis, menkes disease, meralgia paresthetica, metaphyseal dysostosis-intellectual disability-conductive deafness syndrome, methionine adenosyltransferase deficiency, methylcobalamin deficiency cbl g type, methylmalonic acidemia with homocystinuria type cbic, mgat2-cdg (cdg-iia), micro syndrome, microbrachycephaly ptosis cleft lip, microcephalic osteodysplastic primordial dwarfism type 1, microcephalic osteodysplastic primordial dwarfism type 2, microcephalic primordial dwarfism, (e.g., Montreal type, Toriello type), microcephaly, microcephaly autosomal dominant, microcephaly brain defect spasticity hypernatremia, microcephaly cervical spine fusion anomalies, microcephaly deafness syndrome, microcephaly glomerulonephritis marfanoid habitus, microcephaly microcornea syndrome, microcephaly-cardiomyopathy, Microduplication Xp11.22-p11.23 syndrome, microphthalmia syndromic 10, microphthalmia syndromic 4, microphthalmia syndromic 8, microphthalmia with linear skin defects syndrome, microscopic polyangiitis, migraine (e.g. with brainstem aura), mild phenylketonuria, Miller-Dieker syndrome, Miller-Fisher syndrome, minicore myopathy with external ophthalmoplegia, mitochondrial complex i deficiency, mitochondrial complex II deficiency, mitochondrial DNA depletion syndrome, encephalomyopathic form with methylmalonic aciduria, mitochondrial DNA-associated Leigh syndrome, mitochondrial encephalomyopathy lactic acidosis and stroke-like episodes, mitochondrial membrane protein-associated neurodegeneration, mitochondrial myopathy and sideroblastic anemia, mitochondrial myopathy with diabetes, mitochondrial myopathy with lactic acidosis, mitochondrial neurogastrointestinal encephalopathy syndrome, mitochondrial trifunctional protein deficiency, mixed connective tissue disease, Miyoshi myopathy, Moebius syndrome, MOGS-CDG (CDG-IIb), Mohr-Tranebjaerg syndrome, molybdenum cofactor deficiency, monoamine oxidase A deficiency, Morse-Rawnsley-Sargent syndrome, Morvan's fibrillary chorea, Mousa Al din Al Nassar syndrome, Moyamoya disease, MPDU1-CDG (CDG-If), MPI-CDG (CDG-Ib), MPV17-related hepatocerebral mitochondrial DNA depletion syndrome, mucolipidosis type 4, mucopolysaccharidosis type III, mucopolysaccharidosis type IIIA, mucopolysaccharidosis type IIIB, mucopolysaccharidosis type IIIC, mucopolysaccharidosis type IIID, multifocal motor neuropathy, multiple congenital anomalies-hypotonia-seizures syndrome, multiple congenital anomalies-hypotonia-seizures syndrome type 2, multiple myeloma, multiple sulfatase deficiency, multiple system atrophy, multiple system atrophy, multisystemic smooth muscle dysfunction syndrome, muscle eye brain disease, muscular dystrophy white matter spongiosis, megaconial type muscular dystrophy, Muscular phosphorylase kinase deficiency, Musculocontractural Ehlers-Danlos syndrome, Myasthenia gravis, Myelitis, Myelocerebellar disorder, Myelomeningocele, MYH7-related scapuloperoneal myopathy, Myhre syndrome, Myoclonic epilepsy with ragged red fibers, Myoclonus cerebellar ataxia deafness, Myoclonus-dystonia, Myoglobinuria recurrent, Myopathy with extrapyramidal signs, Myosin storage myopathy, Myotonia congenita, Myotonic dystrophy type 1, Myotonic dystrophy type 2, N syndrome, Nance-Horan syndrome, Narcolepsy, NBIA/DYT/PARK-PLA2G6, Necrotizing autoimmune myopathy, Neonatal adrenoleukodystrophy, Neonatal meningitis, Neonatal progeroid syndrome, Neu Laxova syndrome, Neuroblastoma, Neurocutaneous melanosis, Neurofaciodigitorenal syndrome, Neuroferritinopathy, Neurofibromatosis type 1, Neurofibromatosis type 2, Neuroleptic malignant syndrome, Neuromyelitis optica spectrum disorder, Neuronal ceroid lipofuscinosis, Neuronal ceroid lipofuscinosis 10, Neuronal ceroid lipofuscinosis 2, Neuronal ceroid lipofuscinosis 3, Neuronal ceroid lipofuscinosis 5, Neuronal ceroid lipofuscinosis 6, Neuronal ceroid lipofuscinosis 7, Neuronal ceroid lipofuscinosis 9, Neuronal intranuclear inclusion disease, Neuropathic pain, Neuropathy ataxia retinitis pigmentosa syndrome, Neuropathy, distal hereditary motor, Jerash type, Neuropathy, hereditary motor and sensory, Okinawa type, Neuropathy, hereditary motor and sensory, Russe type, Neutral lipid storage disease with myopathy, Nevoid basal cell carcinoma syndrome, New-onset refractory status epilepticus, Nicolaides-Baraitser syndrome, Niemann-Pick disease type A, Niemann-Pick disease type B, Niemann-Pick disease type C1, Niemann-Pick disease type C2, Non-sleep wake disorder, Nondystrophic myotonia, Noonan syndrome, Norrie disease, Northern epilepsy, Oculocerebrocutaneous syndrome, Oculofaciocardiodental syndrome, Oculopharyngeal muscular dystrophy, Oculopharyngodistal myopathy, Okamoto syndrome, Olfactory neuroblastoma, Oligoastrocytoma, Oligodendroglioma, Oliver syndrome, Olivopontocerebellar atrophy, Omphalocele cleft palate syndrome lethal, OPHN1 syndrome, Opsoclonus-myoclonus syndrome, Optic atrophy 2, Optic pathway glioma, Ornithine transcarbamylase deficiency, Orofaciodigital syndrome 1, Orofaciodigital syndrome 10, Orofaciodigital syndrome 2, Orofaciodigital syndrome 3, Orofaciodigital syndrome 4, Orofaciodigital syndrome 5, Orofaciodigital syndrome 6, Orthostatic intolerance due to NET deficiency, Osteopenia and sparse hair, Osteoporosis-pseudoglioma syndrome, Oto-palato-digital syndrome type 1, Oto-palato-digital syndrome type 2, Ouvrier Billson syndrome, Pachygyria-intellectual disability-epilepsy syndrome, PACS1-related syndrome, painful orbital and systemic neurofibromas-marfanoid habitus syndrome, Pallidopyramidal syndrome, Pallister W syndrome, Pallister-Killian mosaic syndrome, Pantothenate kinase-associated neurodegeneration, paralysis agitans, paralysis juvenile, paralysis of Hunt, Paramyotonia congenita, Paraneoplastic/autoimmune (anti-Hu-associated) neuropathy, Parkinson, Parkinson disease type 3, Parkinson disease type 9, Paroxysmal exertion-induced dyskinesia, Paroxysmal extreme pain disorder, Paroxysmal hemicrania, Paroxysmal kinesigenic choreoathetosis, Paroxysomal nonkinesigenic dyskinesia, Parsonage Turner syndrome, Partington syndrome, PCDH19-related female-limited epilepsy, Pediatric autoimmune neuropsychiatric disorders associated with Streptococcus infections, PEHO syndrome, Pelizaeus-Merzbacher disease, Periventricular heterotopia, Periventricular leukomalacia, Perry syndrome, Peters plus syndrome, Pfeiffer Mayer syndrome, Pfeiffer Palm Teller syndrome, Pfeiffer-type cardiocranial syndrome, PGM3-CDG, PHACE syndrome, Phosphoglycerate kinase deficiency, Phosphoglycerate mutase deficiency, Phosphoserine aminotransferase deficiency, Photosensitive epilepsy, Pitt-Hopkins syndrome, Pitt-Hopkins-like syndrome, Plasmacytoma, Pleomorphic xanthoastrocytoma, PMM2-CDG (CDG-Ia), POEMS syndrome, Poliomyelitis, POLR3-Related Leukodystrophy, Polyarteritis nodosa, Polycystic lipomembranous osteodysplasia with sclerosing leukoencephalopathy, Polyneuropathy-intellectual disability-acromicria-premature menopause syndrome, Pontine tegmental cap dysplasia, Pontocerebellar hypoplasia, Pontocerebellar hypoplasia type 1, Pontocerebellar hypoplasia type 2, Pontocerebellar hypoplasia type 3, Pontocerebellar hypoplasia type 4, Pontocerebellar hypoplasia type 5, Pontocerebellar hypoplasia type 6, post-Polio syndrome, Porphyria, posterior column ataxia, posterior column ataxia with retinitis pigmentosa, postnatal progressive microcephaly, postnatal seizures, and postnatal brain atrophy, Potassium aggravated myotonia, Potocki-Lupski syndrome, PPM-X syndrome, Prader-Willi habitus, Primary amebic meningoencephalitis, Primary angiitis of the central nervous system, Primary basilar impression, Primary carnitine deficiency, Primary central nervous system lymphoma, Primary Familial Brain Calcification, Primary lateral sclerosis, Primary melanoma of the central nervous system, Primary orthostatic tremor, Primary progressive aphasia, Primrose syndrome, Progressive bulbar palsy, Progressive encephalomyelitis with rigidity and myoclonus, Progressive external ophthalmoplegia, autosomal recessive 1, progressive hemifacial atrophy, progressive non-fluent aphasia, Progressive Supranuclear Palsy, prolidase deficiency, Proteus syndrome, Proud syndrome, pseudoaminopterin syndrome, Pseudocholinesterase deficiency, pseudoneonatal adrenoleukodystrophy, pseudoprogeria syndrome, pseudotrisomy 13 syndrome, pseudoxanthoma elasticum, Pudendal Neuralgia, Pure autonomic failure, pyridoxal 5′-phosphate-dependent epilepsy, pyridoxine-dependent epilepsy, pyruvate dehydrogenase phosphatase deficiency, Qazi Markouizos syndrome, Radiation induced brachial plexopathy, Ramos Arroyo Clark syndrome, Rapid-onset dystonia-parkinsonism, Rasmussen encephalitis, Reardon Wilson Cavanagh syndrome, Reducing body myopathy, Refsum disease, Renal dysplasia-limb defects syndrome, Renier Gabreels Jasper syndrome, Restless legs syndrome, Retinal arterial macroaneurysm with supravalvular pulmonic stenosis, Retinal vasculopathy with cerebral leukodystrophy, Rett syndrome, Reversible cerebral vasoconstriction syndrome, RFT1-CDG (CDG-In), Rhabdoid tumor, Rhizomelic chondrodysplasia punctata type 1, Riboflavin transporter deficiency, Richards-Rundle syndrome, Richieri Costa Da Silva syndrome, Rigid spine syndrome, Ring chromosome 10, Ring chromosome 14, Ring chromosome 20, Rippling muscle disease, RNAse T2-deficient leukoencephalopathy, Roussy Levy syndrome, RRM2B-related mitochondrial DNA depletion syndrome, Ruvalcaba syndrome, Salla disease, Sandhoff disease, Sandifer syndrome, Sarcoidosis induced neuropathy, Say Barber Miller syndrome, Say Meyer syndrome, Scapuloperoneal syndrome, SCARF syndrome, Schaaf-Yang syndrome, Scheie syndrome, Schimke immunoosseous dysplasia, Schindler disease type 1, Schinzel Giedion syndrome, Schisis association, Schizencephaly, Schwannomatosis, Schwartz Jampel syndrome, Scott Bryant Graham syndrome, Seaver Cassidy syndrome, Seckel syndrome, Semantic dementia, Sensory ataxic neuropathy, Sepiapterin reductase deficiency, Septo-optic dysplasia spectrum, SeSAME syndrome, SETBP1 disorder, severe congenital nemaline myopathy, severe intellectual disability-progressive spastic diplegia syndrome, Gustavson type severe X-linked intellectual disability, Shapiro syndrome, Short-chain acyl-CoA dehydrogenase deficiency, Shprintzen omphalocele syndrome, Shprintzen-Goldberg craniosynostosis syndrome, Sialidosis type 1, Sialidosis, type II, Sickle cell anemia, Simpson-Golabi-Behmel syndrome, Single upper central incisor, Sjogren-Larsson syndrome, SLC35A1-CDG (CDG-IIf), SLC35A2-CDG, SLC35C1-CDG (CDG-IIc), Slow-channel congenital myasthenic syndrome, Smith-Fineman-Myers syndrome, Smith-Lemli-Opitz syndrome, Smith-Magenis syndrome, Sneddon syndrome, Snyder-Robinson syndrome, Sonoda syndrome, spasmodic dysphonia, spastic ataxia charlevoix-saguenay type, spastic diplegia cerebral palsy, spastic diplegia infantile type, spastic paraplegia 1, spastic paraplegia 10, spastic paraplegia 11, spastic paraplegia 12, spastic paraplegia 13, spastic paraplegia 14, spastic paraplegia 15, spastic paraplegia 16, spastic paraplegia 17, spastic paraplegia 18, spastic paraplegia 19, spastic paraplegia 2, spastic paraplegia 23, spastic paraplegia 24, spastic paraplegia 25, spastic paraplegia 26, spastic paraplegia 29, spastic paraplegia 3, spastic paraplegia 31, spastic paraplegia 32, spastic paraplegia 39, spastic paraplegia 4, spastic paraplegia 51, spastic paraplegia 5a, spastic paraplegia 6, spastic paraplegia 7, spastic paraplegia 8, spastic paraplegia 9, spastic paraplegia facial cutaneous lesions, Spastic paraplegia-epilepsy-intellectual disability syndrome, Spastic paraplegia-glaucoma-intellectual disability syndrome, Spastic tetraplegia-retinitis pigmentosa-intellectual disability syndrome, spastic tetraplegia-thin corpus callosum-progressive postnatal microcephaly syndrome, Spina bifida occulta, spinal atrophy ophthalmoplegia pyramidal syndrome, spinal meningioma, spinal muscular atrophy 1, spinal muscular atrophy type 2, spinal muscular atrophy type 3, spinal muscular atrophy-progressive myoclonic epilepsy syndrome, spinal shock, spinocerebellar ataxia, spinocerebellar ataxia 1, spinocerebellar ataxia 10, spinocerebellar ataxia 11, spinocerebellar ataxia 12, spinocerebellar ataxia 13, spinocerebellar ataxia 14, spinocerebellar ataxia 15, spinocerebellar ataxia 17, spinocerebellar ataxia 18, spinocerebellar ataxia 19 and 22, spinocerebellar ataxia 2, spinocerebellar ataxia 20, spinocerebellar ataxia 21, spinocerebellar ataxia 23, spinocerebellar ataxia 25, spinocerebellar ataxia 26, spinocerebellar ataxia 27, spinocerebellar ataxia 28, spinocerebellar ataxia 29, spinocerebellar ataxia 3, spinocerebellar ataxia 30, spinocerebellar ataxia 31, spinocerebellar ataxia 34, spinocerebellar ataxia 4, spinocerebellar ataxia 5, spinocerebellar ataxia 7, spinocerebellar ataxia 8, spinocerebellar ataxia 9, spinocerebellar ataxia autosomal recessive 3, spinocerebellar ataxia autosomal recessive 4, spinocerebellar ataxia autosomal recessive 5, spinocerebellar ataxia autosomal recessive 6, spinocerebellar ataxia autosomal recessive 7, spinocerebellar ataxia autosomal recessive 8, spinocerebellar ataxia type 6, spinocerebellar ataxia with axonal neuropathy type 1, spinocerebellar ataxia with dysmorphism, spinocerebellar ataxia x-linked type 2, spinocerebellar ataxia x-linked type 3, spinocerebellar ataxia x-linked type 4, spinocerebellar degeneration and corneal dystrophy, split hand urinary anomalies spina bifida, split spinal cord malformation, spondyloepiphyseal dysplasia congenita, SRD5A3-CDG (CDG-Iq), SSR4-CDG, STAC3 Disorder, Status epilepticus, Steinfeld syndrome, Stiff person syndrome, Stocco dos Santos syndrome, Striatonigral degeneration infantile, Sturge-Weber syndrome, subacute sclerosing panencephalitis, subcortical band heterotopia, subependymal giant cell astrocytoma, Subependymoma, Succinic semialdehyde dehydrogenase deficiency, Susac syndrome, Symmetrical thalamic calcifications, Syndromic X-linked intellectual disability 7, Tangier disease, TANGO2-Related Metabolic Encephalopathy and Arrhythmias, Tarlov cysts, Tay-Sachs disease, Tel Hashomer camptodactyly syndrome, Telfer Sugar Jaeger syndrome, Temple syndrome, Temple-Baraitser syndrome, Temporal epilepsy, Temtamy syndrome, Tethered cord syndrome, Thoracic dysplasia hydrocephalus syndrome, Thoracic outlet syndromes, Thyrotoxic periodic paralysis, TMEM165-CDG (CDG-IIk), Toriello-Carey syndrome, Tourette syndrome, Toxic neuropathies (e.g. alcoholic neuropathy, chemotherapy-induced neuropathy), Tranebjaerg Svejgaard syndrome, Transverse myelitis, Trichinosis, Trichorhinophalangeal syndrome type 2, Trigeminal neuralgia, Triosephosphate isomerase deficiency, Triple A syndrome, Troyer syndrome, Tuberous sclerosis complex, Tubular aggregate myopathy, Tumefactive multiple sclerosis, Typical congenital nemaline myopathy, Tyrosine hydroxylase deficiency, Tyrosinemia type 1, Ullrich congenital muscular dystrophy, Unverricht-Lundborg disease, Van Benthem-Driessen-Hanveld syndrome, Van Den Bosch syndrome, Variant Creutzfeldt-Jakob disease, Variegate porphyria, Vasculitis induced neuropathy, Vein of Galen aneurysm, Vici syndrome, Viljoen Kallis Voges syndrome, Vincristine induced neuropathy, Visual snow syndrome, Vitamin B6 induced neuropathy, VLCAD deficiency, Vogt-Koyanagi-Harada disease, Von Hippel-Lindau disease, Walker-Warburg syndrome, Weaver syndrome, Welander distal myopathy, Wernicke-Korsakoff syndrome, West syndrome, Whipple disease, White matter hypoplasia-corpus callosum agenesis-intellectual disability syndrome, Wiedemann Oldigs Oppermann syndrome, Williams syndrome, Wilson disease, Wilson-Turner syndrome, Wolf-Hirschhorn syndrome, Wolman disease, Woodhouse Sakati syndrome, Worster Drought syndrome, Wrinkly skin syndrome, Wyburn-Mason syndrome, Xeroderma pigmentosum, Xia-Gibbs syndrome, XK aprosencephaly, X-linked cerebral adrenoleukodystrophy, X-linked Charcot-Marie-Tooth disease type 1, X-linked Charcot-Marie-Tooth disease type 1A, X-linked Charcot-Marie-Tooth disease type 2, X-linked Charcot-Marie-Tooth disease type 3, X-linked Charcot-Marie-Tooth disease type 4, X-linked Charcot-Marie-Tooth disease type 5, X-linked Charcot-Marie-Tooth disease type 6, X-linked complicated corpus callosum agenesis, X-linked complicated spastic paraplegia type 1, X-linked creatine deficiency, X-linked dystonia-parkinsonism/Lubag, X-linked hereditary sensory and autonomic neuropathy with deafness, X-linked intellectual disability—corpus callosum agenesis—spastic quadriparesis, X-linked intellectual disability—short stature—obesity, X-linked intellectual disability, Najm type, X-linked intellectual disability, Schimke type, Siderius type X-linked intellectual disability, Turner type X-linked intellectual disability, X-linked intellectual disability-dysmorphism-cerebral atrophy syndrome, X-linked intellectual disability-plagiocephaly syndrome, X-linked lissencephaly with abnormal genitalia, X-linked myopathy with excessive autophagy, X-linked myotubular myopathy, X-linked non-specific intellectual disability, X-linked periventricular heterotopia, X-linked skeletal dysplasia-intellectual disability syndrome, Zechi Ceide syndrome, Zellweger syndrome, and ZTTK syndrome. In some embodiments, the disease or disorder of the nervous system is a psychiatric disorder. In some embodiments, the disease or disorder of the nervous system is a disease or disorder classified according to the DSM-V (American Psychiatric Association, & American Psychiatric Association, 2013, Diagnostic and statistical manual of mental disorders: DSM-5. Arlington, VA.). In some embodiments, the disease or disorder of the nervous system is a disease or disorder of the central nervous system. In some embodiments, the disease or disorder of the nervous system is an inflammatory disease or disorder of the nervous system. In some embodiments, the disease or disorder of the nervous system described herein is a disease or disorder selected from the group consisting of dementia, multiple sclerosis, amyotrophic lateral sclerosis, Alzheimer's disease, Parkinson's disease, Huntington's disease, frontotemporal lobar dementia, ataxia-teleangiectasia, multiple system atrophy, progressive supranuclear palsy, Krabbe's disease, agenesis of the corpus callosum associated with peripheral neuropathy, Duchenne muscular dystrophy, Guillain-Barré syndrome, Charcot-Marie-Tooth disease Type 1A, hereditary neuropathy with liability to pressure palsies, diabetic neuropathy, toxic neuropathies, age-related peripheral neuropathy, epilepsy, sleep disorders, encephalopathy and neuropathic pain. In some embodiments, the disease or disorder of the nervous system is a neurodegenerative disease or disorder. The term “neurodegenerative disease or disorder”, as used herein, refers to a group of disease or disorders of the nervous system which are characterised by damage and/or death of neuronal subtypes. In some embodiments, the neurodegenerative disease or disorder described herein is at least one disease or disorder selected from the group of dementia, Alzheimer's disease, Parkinson's disease, amyotrophic lateral sclerosis, multiple sclerosis, Huntington's disease, and prion disease. In some embodiments, the disease or disorder of the nervous system described herein, is a toxin and/or drug-induced neuropathy. In some embodiments, the drug-induced neuropathy described herein is induced by, partially induced by or suspected to be induced by at least one agent selected from the group consisting of chemotherapeutic agents, TNF-alpha inhibitors, antiretroviral agents, cardiac medications, statins and antibiotics.

In some embodiments, the drug-induced neuropathy described herein is induced by, partially induced by or suspected to be induced by at least one agent selected from the group consisting of thalidomide, disulfiram, pyridoxine, colchicine, phenytoin, lithium, chloroquine, hydroxychloroquine, cisplatin, oxaliplatin, taxane, vinca alkaloids, bortezomib, suramin, misonidazole, einfliximab, etanercept, zalcitabine, didanosine, stavudine, amiodarone, perhexiline, metronidazole, dapsone, podophyllin, fluoroquinolones, isoniazid and nitrofurantoin.

In some embodiments, the toxin-induced neuropathy described herein is induced by, partially induced by or suspected to be induced by at least one agent selected from the group consisting of organic solvents, heavy metals and organophosphates.

In some embodiments, the toxin and/or drug-induced neuropathy described herein is induced by, partially induced by or suspected to be induced by alcohol and/or cigarette smoke.

In some embodiments, the toxin and/or drug-induced neuropathy described herein is characterized by at least one selected from the group of dorsal root ganglion toxicity, microtubular axon transport function abnormalities, voltage gated abnormalities, sodium channel abnormalities and demyelination.

An “effective amount” of an agent, e.g., a therapeutic agent, refers to an amount effective, at dosages and for periods of time necessary, to achieve the desired therapeutic or prophylactic result. Furthermore, the effective amount may depend on the individual patient's history, age, weight, family history, genetic makeup, stage of the thyroid-related autoimmune disease, the types of preceding or concomitant treatments, if any, and other individual characteristics of the subject to be treated.

In some cases, an effective amount of the composition of the invention can be any amount that reduces the severity, or occurrence, of symptoms of the disease, disorder and/or condition to be treated without producing significant toxicity to the subject. In some cases, an effective amount of the pharmaceutical composition of the invention can be any amount that reduces the number of diseased cells (e.g., dysregulated immune cells), autoantibodies, and/or other disease markers (e.g. cytokines) without producing significant toxicity to the subject.

The effective amount of the pharmaceutical composition of the invention (and any additional therapeutic agent) can remain constant or can be adjusted as a sliding scale or variable dose depending on the subject's response to treatment. In some cases, the frequency of administration can be any frequency that reduces the severity, or occurrence, of symptoms of the disease, disorder and/or condition to be treated without producing significant toxicity to the subject. Various factors can influence the actual effective amount used for a particular application. For example, the frequency of administration, duration of treatment, use of multiple treatment agents, route of administration, and severity of the disease, disorder and/or condition may require an increase or decrease in the actual effective amount administered.

The terms “peptide”, “protein”, “polypeptide”, “polypeptidic” and “peptidic” are used herein interchangeably to designate a series of amino acid residues connected to the other by peptide bonds between the alpha-amino and carboxy groups of adjacent residues.

The term “Alpha1-Antitrypsin protein” or “AAT”, as used herein refers to a protein with an amino acid sequence as defined by the SEQ ID NO: 1 or a nucleotide sequence encoding a protein with an amino acid sequence as defined by the SEQ ID NO: 1. In some embodiments, the AAT described herein is a protein, peptide or polypeptide. AAT protein can be obtained by isolation from blood (e.g. human blood) or can be produced recombinantly.

The term “variant” refers to a protein, peptide or polypeptide having an amino acid sequence that differ to some extent from the AAT native sequence peptide, that is an amino acid sequence that vary from the AAT native sequence by amino acid substitutions, whereby one or more amino acids are substituted by another with same characteristics and conformational roles. Preferably, a variant described herein is at least 80%, 81%, 82%, 83%, 84%, 85%, 86%, 87%, 88%, 89%, 90%, 91%, 92%, 93%, 94%, 95%, 96%, 97%, 98%, or 99% homologous to amino acids of SEQ ID NO: 1. The amino acid sequence variants can have substitutions, deletions, and/or insertions at certain positions within the amino acid sequence of the native amino acid sequence, e.g. at the N- or C-terminal sequence or within the amino acid sequence. Substitutions can also be conservative, in this case, the conservative amino acid substitutions are herein defined as exchanges within one of the following five groups:
I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gin
IV. Large, aromatic residues: Phe, Tyr, Trp
V. Large, aliphatic, nonpolar residues: Met, Leu, lie, Val, Cys.

The term “isoform”, as used herein, refers to a splice variant resulting from alternative splicing of the AAT mRNA. Isoforms of AAT are known in the art (see e.g. Matsuda, E., Ishizaki, R., Taira, T., Iguchi-Ariga, S. M., & Ariga, H., 2005, Biological & pharmaceutical bulletin, 28(5), 898-901).

The term “fragment”, as used herein, refers to a sequence containing less amino acids in length than the AAT protein and/or isoform thereof, in particular less amino acids than the sequence of AAT as set forth in SEQ ID NO:1. The fragment is preferably a functional fragment, e.g. a fragment with the same biological activities as the AAT protein as set forth in SEQ ID NO:1. The functional fragment preferably derived from the AAT protein as set forth in SEQ ID NO:1. Any AAT fragment can be used as long as it exhibits the same properties or substantially the same, i.e. is biologically active, as the native AAT sequence from which it derives. In some embodiments, the fragment described herein has the same or substantially the same inhibitory properties as AAT for one or more human neutrophil serine proteases, preferably the fragment has second-order constants of association of the AAT fragment with NE of at least about 6.5×10⁷, with PR₃ of at least about 8.1×10⁶, and/or with CG of at least about 4.1×10⁵ M⁻¹ s⁻¹, respectively (for the measurement method see e.g. Beatty, K., et al., 1980, J. Biol. Chem. 255, 3931-3934.; Rao, N. V., et al., 1991, Structural and functional properties. J. Biol. Chem. 266, 9540-9548.).

Preferably, the (functional) fragment shares about 5 consecutive amino-acids, at least about 7 consecutive amino-acids, at least about 15 consecutive amino-acids, at least about 20 consecutive amino-acids, at least about 25 consecutive amino-acids, at least about 20 consecutive amino-acids, at least about 30 consecutive amino-acids, at least about 35 consecutive amino-acids, at least about 40 consecutive amino-acids, at least about 45 consecutive amino-acids, at least about 50 consecutive amino-acids, at least about 55 consecutive amino-acids, at least about 60 consecutive amino-acids, at least about 100 consecutive amino-acids, at least about 150 consecutive amino-acids, at least about 200 consecutive amino-acids, at least about 300 consecutive amino-acids, or more of the native human AAT amino acid sequence as set forth in SEQ ID NO:1. In some embodiments, the (functional) fragment described herein, comprises an expression optimized signal protein.

In some aspects, the amino acid sequence of AAT, the variant, isoform or fragment thereof is identical to a corresponding amino acid sequence in SEQ ID NO: 1.

To date, the natural inhibitor for a broad set of proteases alpha-1-Antitrypsin (AAT) has been successfully used to attenuate inflammation, this in different type of human tissue (Bergin, David A., et al., 2021, Archivum immunologiae et therapiae experimentalis 60.2: 81-97).

The inventors found, that AAT can reduce neuronal pathology pathways (FIG. 2-4, Table 2-11). This reduction of neuronal pathology pathways was observed in resting cells (FIG. 4B) and stimulated cells (FIG. 4C) and is therefore useful in preventing and/or treating diseases or disorders of the nervous system and symptoms thereof.

TACE activity modulation is involved in myelin regulation and as an inflammation hallmark of the acquired form of neuropathies.

The inventors found, that AAT is able to inhibit TACE in a dose-dependent manner which, without being bound by theory, rescues myelin production by SCs, and thus subsequently prevents, or slowing, and/or reverses the progression of diseases and/or disorders of the nervous system.

Excitingly, AAT offers some hope to a disease that currently has no curative treatment available for the underlying genetic process at this time, and no treatment consistently found to be effective in slowing the progression of the disease process.

Accordingly, the invention is at least in part based on the finding that AAT is useful in treating disease or disorders of the nervous system as described herein.

In certain embodiments, the invention relates to a vector comprising a nucleic acid sequence encoding an AAT protein for use in the treatment and/or prevention of a disease or disorder of the nervous system.

The term “vector”, as used herein, refers to a viral vector or to a nucleic acid (DNA or RNA) molecule such as a plasmid or other vehicle, which contains one or more heterologous nucleic acid sequence(s) of the invention and, preferably, is designed for transfer between different host cells. The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

The terms “nucleic acid”, “polynucleotide”, and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity; i.e., an analogue of A will base-pair with T.

The use of a vector as described herein can reduce limitations of proteins such as blood brain barrier penetration and/or enzymatic degradation of AAT. As such, a vector can be implemented in delivery systems such as cells that deliver the AAT to neuronal cells such as neuronal cells of the brain.

Accordingly, the invention is at least in part based on the finding that a vector as described herein is useful in the treatment and/or prevention of diseases or disorders of the nervous system.

In certain embodiments, the invention relates to a genetically modified cell comprising a nucleic acid sequence encoding an AAT protein for use in the treatment and/or prevention of a disease or disorder of the nervous system.

The term “genetically modified cell”, as used herein, refers to a cell modified by means of genetic engineering. In some embodiments, the cell is an immune effector cell. The term as used herein “engineered” and other grammatical forms thereof may refer to one or more changes of nucleic acids, such as nucleic acids within the genome of an organism. The term “engineered” may refer to a change, addition and/or deletion of a gene. Engineered cells can also refer to cells that contain added, deleted, and/or changed genes.

In some embodiments, the genetically modified cell described herein include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally genetically modified cell are included herein.

In some embodiments, the invention relates to a composition comprising the genetically modified cell described herein instead of or in addition to the AAT protein, variant or isoform thereof. The genetically modified cell described herein may therefore be used in cell therapy to deliver AAT in a subject or to a tissue/organ of a subject.

The use of a genetically modified cell as described herein can reduce limitations of proteins such as blood brain barrier penetration and/or enzymatic degradation of AAT. As such, a vector can be implemented in delivery systems such as cells that deliver the AAT to neuronal cells such as neuronal cells of the brain.

Accordingly, the invention is at least in part based on the finding that a genetically modified cell as described herein can improve the prevention and/or therapy of diseases or disorders of the nervous system.

In certain embodiments, the invention relates to the composition for use of the invention, the vector for use of the invention or the genetically modified cell for use of the invention, wherein the disease or disorder of the nervous system is an inflammatory disease or disorder of the nervous system.

The term “inflammatory disease or disorder of the nervous system”, as used herein, refers to a disorder or disorder of the nervous system that is characterized by increased inflammation. Inflammation is characterized by a dysregulation of inflammation markers and/or increased immune cell infiltration, activation, proliferation, and/or differentiation in the blood, in a tissue, in an organ and/or in a certain cell-type.

The inflammation of the disease or disorder of the nervous system can be caused for example by physical injury, ionizing radiation, infections (e.g., by pathogens), immune reactions due to hypersensitivity, cancer, chemical irritants, medications, toxins, alcohol, nutrients (e.g., nutrient excess), plaque deposit, toxic metabolites, autoimmunity, aging, microbes, air pollution and/or (passive) smoking.

An inflammation marker is a marker that is indicative for inflammation in a subject. Inflammatory markers include, without limitation, CRP, erythrocyte sedimentation rate (ESR), and procalcitonin (PCT), Interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-33, IL-32, IL-33, IL-35 or IL-36) Tumor necrosis factor (e.g., TNF alpha, TNF beta), Interferon (e.g., interferon alpha, interferon beta, interferon gamma) MIP-I, MCP-I, RANTES, other chemokines and/or other cytokines. An inflammatory marker may also be detectable indirectly, e.g., by detection of an inhibitory factor of an inflammatory marker (e.g., binding factor and/or antagonist). In some embodiments, the inflammatory marker is measured in cells involved in inflammation, in cells affected by cells involved in inflammation, in a tissue, and/or in the blood. In some embodiments, the inflammation marker is indicative for immune cell infiltration, activation, proliferation and/or differentiation. Detection of the inflammation marker or the ratio of two or more inflammation markers is detected outside the normal range. The normal range of inflammation markers and whether a marker (ratio) has to be below or above a threshold to be indicative for inflammation is known to the person skilled in the art. In some embodiments, the inflammation marker is a microglial marker such as a microglial identification, proliferation, accumulation and/or activation marker. In some embodiments, the gene expression level, the RNA transcript level, the protein expression level, the protein activity level and/or the enzymatic activity level of at least one inflammation marker is detected. In some embodiments at least one inflammation marker is detected quantitatively and/or qualitatively.

The inventors found, that AAT (or the vector/genetically modified cell described herein) can reduce neuronal inflammatory pathways (FIG. 2-4, Table 2-11). This reduction of inflammatory pathways was observed in resting cells (FIG. 4B) and stimulated cells (FIG. 4C) and is therefore useful in preventing and/or treating diseases or disorders of the nervous system and symptoms thereof.

Accordingly, the invention is at least in part based on the finding that AAT (or the vector/genetically modified cell described herein) is useful in treating inflammatory diseases or disorders of the nervous system as described herein.

In certain embodiments, the invention relates to the composition for use of the invention, the vector for use of the invention or the genetically modified cell for use of the invention, wherein the inflammatory disease or disorder of the nervous system is a myeloid cell-mediated disease or disorder of the nervous system.

The term “myeloid cell-mediated disease or disorder of the nervous system”, as used herein, refers to a disorder or disorder of the nervous system that is characterized by increased myeloid cell-mediated inflammation. Myeloid cell-mediated inflammation can be detected by any method known in the art, for example by cytokine measurements and/or quantitative and/or qualitative analysis of the myeloid cells (see e.g. Davis, B. M., Salinas-Navarro, M., Cordeiro, M. F. et al., 2017, Sci Rep 7, 1576).

In some embodiments, the myeloid cell-mediated disease or disorder of the nervous system is a disease or disorder, wherein the primary pathology is myeloid cell-mediated inflammation.

In some embodiments, the myeloid cell-mediated disease or disorder of the nervous system is a microglia cell-mediated disease or disorder of the nervous system.

The inventors found, that AAT (or the vector/genetically modified cell described herein) can reduce neuronal myeloid cell-mediated inflammatory pathways (FIG. 2-4, Table 2 to 11). This reduction of myeloid cell-mediated inflammatory pathways was observed in resting cells (FIG. 4B) and stimulated cells (FIG. 4C) and is therefore useful in preventing and/or treating diseases or disorders of the nervous system and symptoms thereof.

Accordingly, the invention is at least in part based on the finding that AAT (or the vector/genetically modified cell described herein) is useful in treating microglia cell-mediated diseases or disorders of the nervous system as described herein.

In certain embodiments, the invention relates to the composition for use of the invention, the vector for use of the invention or the genetically modified cell for use of the invention, wherein the disease or syndrome of the nervous system is a disease or syndrome selected from the group of dementia, multiple sclerosis, amyotrophic lateral sclerosis, Alzheimer's disease, Parkinson's disease and Huntington's disease.

The term “dementia”, as used herein, refers to a cognitive disorder characterized by dementia (i.e., general deterioration or progressive decline of cognitive abilities or dementia-like symptoms). Dementia disorders are often associated with, or caused by, one or more aberrant processes in the brain or central nervous system (e.g. neurodegeneration). Dementia disorders commonly progress from mild through severe stages and interfere with the ability of a subject to function independently in everyday life. Dementia may be classified as cortical or subcortical depending on the area of the brain affected. Dementia disorders do not include disorders characterized by a loss of consciousness (as in delirium) or depression, or other functional mental disorders (pseudodementia). Dementia disorders include the irreversible dementias such as those associated with neurodegenerative diseases such Alzheimer's disease, vascular dementia, Lewy body dementia, Jakob-Creutzfeldt disease, Pick's disease, progressive supranuclear palsy, Frontal lobe dementia, idiopathic basal ganglia calcification, Huntington disease, multiple sclerosis, and Parkinson's disease, as well as reversible dementias due to trauma (posttraumatic encephalopathy), intracranial tumors (primary or metastatic), subdural hematomas, metabolic and endocrinologic conditions (hypo- and hyperthyroidism, Wilson's disease, uremic encephalopathy, dialysis dementia, anoxic and post-anoxic dementia, and chronic electrolyte disturbances), deficiency states (Vitamin B12 deficiency and pellagra (vitamin B6)), infections (AIDS, syphilitic meningoencephalitis, limbic encephalitis, progressive multifocal leukoencephalopathy, fungal infections, tuberculosis), and chronic exposure to alcohol, aluminum, heavy metals (arsenic, lead, mercury, manganese), or prescription drugs (anticholinergics, sedatives, barbiturates, etc.).

The term “multiple sclerosis”, as used herein, refers to a disease or disorder characterized by inflammation, demyelination, oligodendrocyte death, membrane damage and axonal death. In some embodiments, the multiple sclerosis described herein refers to relapsing/remitting multiple sclerosis or progressive multiple sclerosis. In some embodiments, multiple sclerosis is at least one of the four main multiple sclerosis varieties as defined in an international survey of neurologists (Lublin and Reingold, 1996, Neurology 46(4):907-11), which are namely, relapsing/remitting multiple sclerosis, secondary progressive multiple sclerosis, progressive/relapsing multiple sclerosis, or primary progressive multiple sclerosis (PPMS).

In some embodiments, the multiple sclerosis described herein refers to symptoms of multiple sclerosis which comprise vision problems, dizziness, vertigo, sensory dysfunction, weakness, problems with coordination, loss of balance, fatigue, pain, neurocognitive deficits, mental health deficits, bladder dysfunction, bowel dysfunction, sexual dysfunction, heat sensitivity.

The term “Huntington's disease”, as used herein, refers to a neurodegenerative disease caused by a tri-nucleotide repeat expansion (e.g., CAG, which is translated into a poly-Glutamine, or PolyQ, tract) in the HTT gene that results in production of pathogenic mutant huntingtin protein (HTT, or mHTT). In some embodiments, mutant huntingtin protein accelerates the rate of neuronal cell death in certain regions of the brain. In some embodiments, the Huntington's disease described herein refers to symptoms of Huntington's disease which comprise impaired motorfunction, cognitive impairment, depression, anxiety, movement disturbances, chorea, rigidity, muscle contracture (dystonia), slow eye movements or abnormal eye movements, impaired gait, altered posture, impaired balance, unintended weight loss, sleep rhythm disturbances, circadian rhythm disturbances and autonomic nervous system dysfunction.

The term “amyotrophic lateral sclerosis”, as used herein, refers to a progressive neurodegenerative disease that affects upper motor neurons (motor neurons in the brain) and/or lower motor neurons (motor neurons in the spinal cord) and results in motor neuron death. In some embodiments, amyotrophic lateral sclerosis includes all of the classifications of amyotrophic lateral sclerosis known in the art, including, but not limited to classical amyotrophic lateral sclerosis (typically affecting both lower and upper motor neurons), Primary Lateral Sclerosis (PLS, typically affecting only the upper motor neurons), Progressive Bulbar Palsy (PBP or Bulbar Onset, a version of amyotrophic lateral sclerosis that typically begins with difficulties swallowing, chewing and speaking), Progressive Muscular Atrophy (PMA, typically affecting only the lower motor neurons) and familial amyotrophic lateral sclerosis (a genetic version of amyotrophic lateral sclerosis).

In some embodiments, the term “amyotrophic lateral sclerosis” refers to symptoms of amyotrophic lateral sclerosis, which include, without limitation, progressive weakness, atrophy, fasciculation, hyperreflexia, dysarthria, dysphagia and/or paralysis of respiratory function.

The term “Alzheimer's disease” (AD), as used herein, refers to mental deterioration associated with a specific degenerative brain disease that is characterized by senile plaques, neuritic tangles and progressive neuronal loss which manifests clinically in progressive memory deficits, confusion, behavioral problems, inability to care for oneself and/or gradual physical deterioration.

In some embodiments, subjects suffering Alzheimer's disease are identified using the NINCDS-ADRDA (National Institute of Neurological and Communicative Disorders and the Alzheimer's Disease and Related Disorders Association) criteria:
1) Clinical Dementia Rating (CDR)=1; Mini Mental State Examination (MMSE) between 16 and 24 points and Medial temporal atrophy (determined by Magnetic Resonance Imaging, MRI)>3 points in Scheltens scale. In some embodiments, the term Alzheimer's disease includes all the stages of the disease, including the following stages defined by NINCDS-ADRDA Alzheimer's Criteria for diagnosis in 1984.
2) Definite Alzheimer's disease: The patient meets the criteria for probable Alzheimer's disease and has histopathologic evidence of AD via autopsy or biopsy.
Probable or prodromal Alzheimer's disease: Dementia has been established by clinical and neuropsychological examination. Cognitive impairments also have to be progressive and be present in two or more areas of cognition. The onset of the deficits has been between the ages of 40 and 90 years and finally there must be an absence of other diseases capable of producing a dementia syndrome.
3) Possible or non-prodromal Alzheimer's disease: There is a dementia syndrome with an atypical onset, presentation; and without a known etiology; but no co-morbid diseases capable of producing dementia are believed to be in the origin of it. In some embodiments, the term Alzheimer's disease refers one stage of Alzheimer's disease. In some embodiments, the term Alzheimer's disease refers to two stages of Alzheimer's disease. In some embodiments, the term “Alzheimer's disease” refers to symptoms of Alzheimer's disease, which include without limitation, loss of memory, confusion, difficulty thinking, changes in language, changes in behavior, and/or changes in personality.

The term “Parkinson's disease”, as used herein, refers to a neurological syndrome characterized by a dopamine deficiency, resulting from degenerative, vascular, or inflammatory changes in the basal ganglia of the substantia nigra. Symptoms of Parkinson's disease include, without limitation, the following: rest tremor, cogwheel rigidity, bradykinesia, postural reflex impairment, good response to 1-dopa treatment, the absence of prominent oculomotor palsy, cerebellar or pyramidal signs, amyotrophy, dyspraxia, and/or dysphasia. In a specific embodiment, the present invention is utilized for the treatment of a dopaminergic dysfunction-related syndrome. In some embodiments, Parkinson's disease includes any stage of Parkinson's disease. In some embodiments, the term Parkinson's disease includes the early stage of Parkinson's disease, which refers broadly to the first stages in Parkinson's disease, wherein a person suffering from the disease exhibits mild symptoms that are not disabling, such as an episodic tremor of a single limb (e.g., the hand), and which affect only one side of the body.

In some embodiments, the term Parkinson's disease includes the advanced stage of Parkinson's disease, which refers to a more progressive stage in Parkinson's disease, wherein a person suffering from the disease exhibits symptoms which are typically severe and which may lead to some disability (e.g., tremors encompassing both sides of the body, balance problems, etc.). Symptoms associated with advanced-stage Parkinson's disease may vary significantly in individuals and may take several years to manifest after the initial appearance of the disease.

In some embodiments, the term “Parkinson's disease” refers to symptoms of Parkinson's disease, which include without limitation, tremors (e.g., tremor which is most pronounced during rest), shaking (e.g. trembling of hands, arms, legs, jaw and face), muscular rigidity, lack of postural reflexes, slowing of the voluntary movements, retropulsion, mask-like facial expression, stooped posture, poor balance, poor coordination, bradykinesia, postural instability, and/or gait abnormalities.

Accordingly, the invention is at least in part based on the finding that AAT (or the vector/genetically modified cell described herein) is particularly useful in treating certain diseases or disorders of the nervous system as described herein.

In certain embodiments, the invention relates to the composition for use of the invention, the vector for use of the invention or the genetically modified cell for use of the invention, wherein the disease or disorder of the nervous system is at least one symptom of a disease or disorder of the nervous system selected from the group consisting of: tremor, memory loss, slurred speech, dizziness, change in vision and headache.

In certain embodiments, the invention relates to the composition for use of the invention, the vector for use of the invention, or the genetically modified cell for use of the invention, wherein the disease or disorder of the nervous system is a Schwann cell-mediated disease or disorder.

In certain embodiments, the invention relates to the composition for use of the invention, the vector for use of the invention, or the genetically modified cell for use of the invention, wherein the disease or disorder of the nervous system is a TACE-mediated disease or disorder.

In certain embodiments, the invention relates to the composition for use of the invention, the vector for use of the invention, or the genetically modified cell for use of the invention, wherein the disease or disorder of the nervous system is a disease or disorder of the peripheral nervous system.

The term “disease or disorder of the peripheral nervous system”, as used herein, refers to includes any disease or disorder that substantially affects the peripheral nervous system, preferable any disease or disorder that primarily affects the peripheral nervous system.

In certain embodiments, the invention relates to the composition for use of the invention, the vector for use of the invention or the genetically modified cell for use of the invention, wherein the disease or disorder of the peripheral nervous system is motor and sensory neuropathy of the peripheral nervous system.

In certain embodiments, the invention relates to the composition for use of the invention, the vector for use of the invention or the genetically modified cell for use of the invention, wherein the sensory neuropathy of the peripheral nervous system is an acquired motor and sensory neuropathy of the peripheral nervous system.

Acquired motor and sensory neuropathy of the peripheral nervous system such as acquired demyelinating diseases include, without limitation nerve injury, diabetic peripheral neuropathy, drug-related peripheral neuropathies, leprosy, and inflammatory neuropathies. These neuropathies can affect both myelinated Schwann cells and peripheral axons/neurons.

In certain embodiments, the invention relates to the composition for use of the invention, the vector for use of the invention or the genetically modified cell for use of the invention, wherein the sensory neuropathy of the peripheral nervous system is a Guillain-Barré Syndrome.

In certain embodiments, the invention relates to the composition for use of the invention, the vector for use of the invention or the genetically modified cell for use of the invention, wherein the sensory neuropathy of the peripheral nervous system is a hereditary motor and sensory neuropathy of the peripheral nervous system.

In certain embodiments, the invention relates to the composition for use of the invention, the vector for use of the invention, or the genetically modified cell for use of the invention, wherein the hereditary motor and sensory neuropathy of the peripheral nervous system is Charcot-Marie-Tooth disease or a symptom thereof.

The term “Charcot-Marie-Tooth disease”, as used herein, refers to a hereditary motor and sensory neuropathy of the peripheral nervous system characterized by progressive loss of muscle tissue and/or touch sensation across various parts of the body. In some embodiments, the Charcot-Marie-Tooth disease described herein is at least one subtype selected from the group of CMT1, CMTX, CMT4, CMT2, Severe, early onset CMT, CMT 5, CMT 6, CMT 7, and intermediate CMT.

Symptoms of Charcot-Marie-Tooth disease include, without limitation, weakness in legs, ankles and/or feet, loss of muscle bulk in legs and/or feet, high foot arches, curled toes (hammertoes), decreased ability to run, difficulty lifting foot at the ankle (footdrop), abnormal gait, frequent tripping or falling and decreased sensation or a loss of feeling in legs and/or feet.

In a mouse model for disease and disorders of the peripheral nervous system such as CMT1A the inventors confirmed the finding that AAT is an effective therapy context.

With that great promise is placed on AAT in ameliorating the dysmyelination of axons, allowing myelin sheaths to form properly around axons and by doing so potentially allowing CMT1A patients to live normal and healthy lives.

In certain embodiments, the invention relates to the composition for use of the invention, wherein the AAT protein, a variant, an isoform and/or a fragment thereof is human plasma-extracted.

In certain embodiments, the invention relates to the composition for use of the invention, wherein the alpha1-antitrypsin (AAT) protein, a variant, an isoform and/or a fragment thereof is recombinant alpha1-antitrypsin (rhAAT), a variant, an isoform and/or a fragment thereof.

In certain embodiments, the invention relates to the composition for use of the invention, wherein the composition comprises at least one pharmaceutical carrier.

The term “pharmaceutical carrier”, as used herein, refers to an agent (e.g. a molecule or a cell) that improves drug delivery properties of the composition, the vector and/or the genetically modified cell for use of the invention. In some embodiments the drug delivery property describe herein comprises at least one property selected from the group of penetration ability (e.g. cell-membrane and/or blood brain barrier), site specific delivery (e.g. brain specific delivery), controlled release delivery and stability (e.g., reduction of enzymatic degradation). In some embodiments, the pharmaceutical carrier described herein is an agent selected from the group of delivery cell, liposome, nanoparticle, fusion protein, niosome, nanosphere, micelle, nanocapsule, nanoshell, lipid particle and dendrimer.

In some embodiments the pharmaceutical carrier described herein is a pharmaceutically acceptable diluent or carrier.

In certain embodiments, the invention relates to the composition for use of the invention, wherein the pharmaceutical carrier is a blood-brain barrier permeability enhancer.

The term “blood-brain barrier permeability enhancer”, as used herein, refers to an agent that can be used to deliver the composition, vector and/or genetically modified cell for use of the invention to the nervous system, including the brain and to pass the blood brain barrier. Any strategy known in the art may be used to achieve the enhancement of the blood-brain barrier permeability (see e.g., Salameh, T. S., & Banks, W. A., 2014, Advances in pharmacology, 71, 277-299; Tashima, T., 2020, Receptor-Mediated Transcytosis. Chemical and Pharmaceutical Bulletin, 68(4), 316-32; Pardridge, W. M., 2020, Frontiers in aging neuroscience, 11, 373; Upadhyay, R. K., 2014, BioMed research international).

In some embodiments, the composition for use of the invention is fused to a blood-brain barrier enhancing protein. In some embodiments the blood-brain barrier enhancing protein described herein is at least one full protein, variant, isoform and/or fragment of the protein selected from the group of transferrin, insulin, insulin-like growth factor, low density lipoprotein.

Trojan horse strategies may also be used (see e.g. Pardridge, W. M., 2017, BioDrugs 31, 503-519). In some embodiments, the composition for use of the invention is linked to an antibody or a fragment thereof that binds to an endogenous BBB receptor transporter, such as the insulin receptor or transferrin receptor.

The composition for use of the invention may also be altered to increase lipophilicity and subsequently improve BBB crossing properties (see e.g. Upadhyay, R. K., 2014. BioMed research international, Article ID 869269, 37 pages). In some embodiments, the composition for use of the invention comprises modifications that increase the lipophilicity. In some embodiments the modifications that increase the lipophilicity described herein comprise the addition of at least one hypdrophilic peptide, replacement of sequence parts with at least one hypdrophilic peptide, the addition of lipid moieties, and/or replacement of non-lipid moieties with lipid moieties.

In certain embodiments, the invention relates to the composition for use of the invention, the vector for use of the invention or the genetically modified cell for use of the invention, wherein the composition, the vector or the genetically modified cell is formulated for intracerebral administration, intravenous injection, intravenous infusion, infusion with a dosator pump, inhalation nasal-spray, eye-drops, skin-patches, slow release formulations, ex vivo gene therapy or ex vivo cell-therapy.

The pharmaceutical compositions of the present invention may also be delivered to the patient, by several technologies including DNA injection of nucleic acid encoding the AAT protein, a variant, an isoform and/or a fragment thereof of the invention (also referred to as DNA vaccination) with and without in vivo electroporation, liposome mediated, nanoparticle facilitated, recombinant vectors such as recombinant lentivirus, recombinant adenovirus, and recombinant adenovirus associated virus as described herein.

The compositions may be injected intra venously or locally injected into the brain or spinal cord or electroporated in the tissue of interest.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other embodiments, the subject can be a normal subject. The term does not denote a particular age or sex. Thus, adult, child and new-born subjects, whether male or female, are intended to be covered. Preferably, the subject is a human. Most preferably a human suffering from a disease or syndrome related to a virus infection, preferably a coronavirus infection, more preferably a SARS-CoV-2 infection. In some embodiments, the subject is suffering from a neurological disorder independent of a viral infection.

The term “about,” particularly in reference to a given quantity, is meant to encompass deviations of plus or minus ten (10) percent, preferably 5 percent, even more preferably 2 percent and most preferably 1 percent.

The present invention relates to a composition for use in the treatment and/or prevention of disease or syndrome related to any virus infection in a subject in need thereof, the composition comprising a therapeutically effective amount of an Alpha1-Antitrypsin protein, a variant, an isoform and/or a fragment thereof.

The alpha1-antitrypsin (AAT) protein, a variant, an isoform and/or a fragment thereof can be a plasma-extracted AAT, a variant, an isoform and/or a fragment thereof, in particular a human plasma-extracted AAT, a variant, an isoform and/or a fragment thereof; or a recombinant alpha1-antitrypsin (rhAAT) protein, a variant, an isoform and/or a fragment thereof, preferably the alpha1-antitrypsin (AAT) protein, a variant, an isoform and/or a fragment thereof is a recombinant alpha1-antitrypsin (rhAAT) protein, a variant, an isoform and/or a fragment thereof. The virus infection can be due to a DNA virus (double or single stranded), an RNA virus (single or double stranded, whether positive of negative), a reverse transcribing virus or any emerging virus, whether enveloped or non-enveloped.

In some embodiments of the invention, the composition is used in the treatment and/or prevention of disease or syndrome related to a respiratory virus infection in a subject in need thereof. In some embodiments, the respiratory virus described herein is a virus selected from the group of Rhinovirus, RSV, Parainfluenza, Metapneumovirus, Coronavirus, Enterovirus, Adenovirus, Bocavirus, Polyomavirus, Herpes simplex virus, and Cytomegalovirus.

In some embodiments of the invention, the composition is used in the treatment and/or prevention of disease or syndrome related to a DNA virus infection in a subject in need thereof.

In some embodiments, the DNA virus described herein is selected from the group consisting of Adenovirus, Rhinovirus, RSV, Influenza virus, Parainfluenza virus, Metapneumovirus, Coronavirus, Enterovirus, Adenovirus, Bocavirus, Polyomavirus, Herpes simplex virus, Cytomegalovirus, Bocavirus, Polyomavirus, and Cytomegalovirus.

In some embodiments of the invention, the composition is used in the treatment and/or prevention of disease or syndrome related to an RNA virus infection in a subject in need thereof. The RNA virus may be an enveloped or coated virus or a nonenveloped or naked RNA virus. The RNA virus may be single stranded RNA (ssRNA) virus or a double stranded RNA (dsRNA) virus. The single stranded RNA virus may be a positive sense ssRNA virus or a negative sense ssRNA virus.

In some embodiments, the RNA virus described herein is selected from the group consisting of Rhinovirus, RSV, Influenza virus, Parainfluenza virus, Metapneumovirus, Coronavirus, Enterovirus Adenovirus, Bocavirus, Polyomavirus, Herpes simplex virus, and Cytomegalovirus.

In some embodiments of the invention, the composition is used in the treatment and/or prevention of disease or syndrome related to a Coronavirus infection in a subject in need thereof. In some embodiments, the Coronavirus described herein is a Coronavirus from the genus selected from the group of α-CoV, β-CoV, γ-CoV or δ-CoV. In another specific embodiment, the Coronavirus described herein is of the genus α-CoV or β-CoV. In some embodiments, the Coronavirus described herein is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-CoV or “novel coronavirus 2012”), Severe acute respiratory syndrome coronavirus (SARS-CoV or “SARS-classic”), and Severe acute respiratory syndrome coronavirus 2 (SARS-CoV-2 or “novel coronavirus 2019”). Preferably, the virus infection is an RNA virus infection, most preferably a coronavirus infection due to a coronavirus selected from the non-limiting group comprising MERS-CoV, SARS-CoV and SARS-CoV-2. Most preferably the virus infection is a SARS-CoV-2 infection.

It is to be understood that the invention includes all diseases or syndromes related a virus infection, preferably a coronavirus infection, more preferably a SARS-CoV-2 infection. The disease or syndrome related to a SARS-CoV-2-infection is also referred to as COVID-19. In one embodiment, the disease or syndrome related a virus infection is an inflammation, e.g. of blood vessels throughout the body (e.g. Kawasaki disease), an immune disease (e.g. Grave's disease) and/or a respiratory syndrome, in particular a severe acute respiratory syndrome. The disease or syndrome can be related a virus infection in that the virus infection is associated with the disease or syndrome, prior to the disease or syndrome, contributes to the disease or syndrome and/or causes the disease or syndrome. For example, the virus infection may induce inflammation that directly or indirectly induces a disease or syndrome. The inflammation related to the virus infection may be acute or chronic inflammation. The inflammation related to the virus infection may subsequently persist systemically and/or in a specific organ e.g. the brain and/or induce lasting damages. In some embodiments of the invention, the composition is used in the treatment and/or prevention of an inflammatory disease or syndrome of the nervous system related to a virus infection in a subject in need thereof.

The term “inflammatory disease or syndrome of the nervous system”, as used herein, refers to a disease, a syndrome and/or a condition that is characterized by increased inflammation in the nervous system compared to a healthy reference subject. The disease or syndrome described herein including disease or syndrome of the nervous system is related to a virus. Inflammation is characterized by a dysregulation of inflammation markers and/or increased immune cell infiltration, activation, proliferation, and/or differentiation in the blood and/or the brain. An inflammation marker is a marker that is indicative for inflammation in a subject. In certain embodiments the inflammatory marker described herein is a marker selected from the group of CRP, erythrocyte sedimentation rate (ESR), and procalcitonin (PCT), Interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-33, IL-32, IL-33, IL-35 or IL-36) Tumor necrosis factor (e.g., TNF alpha, TNF beta), Interferon (e.g., interferon gamma) MIP-1, MCP-I, RANTES, other chemokines and/or other cytokines. An inflammatory marker may also be detectable indirectly, e.g., by detection of an inhibitory factor of an inflammatory marker (e.g., binding factor and/or antagonist). In some embodiments, the inflammatory marker is measured in cells involved in inflammation, in cells affected by cells involved in inflammation, in the cerebrospinal fluid, and/or in the blood. In some embodiments, the inflammation marker is indicative for immune cell infiltration, activation, proliferation and/or differentiation. Detection of the inflammation marker or the ratio of two or more inflammation markers is detected outside the normal range. The normal range of inflammation markers and whether a marker (ratio) has to be below or above a threshold to be indicative for inflammation is known to the person skilled in the art. In some embodiments, the gene expression level, the RNA transcript level, the protein expression level, the protein activity level and/or the enzymatic activity level of at least one inflammation marker is detected. In some embodiments at least one inflammation marker is detected quantitatively and/or qualitatively to determine the inflammatory disease or syndrome of the nervous system in a subject in need of treatment and/or prevention.

In some embodiments, the inflammatory disease or syndrome of the nervous system described herein is characterized by acute inflammation, that is the duration of inflammation symptoms typically takes from about a few minutes (e.g., 2, 5, 10, 15, 30, 45 minutes) to a few days (e.g., 2, 3, 5, 7, 10 or 14 days). Acute inflammation typically occurs as a direct result of a stimulus such as virus infection. In some embodiments, the inflammatory disease or syndrome of the nervous system is characterized by chronic inflammation, that is the duration of symptoms of inflammation typically take at least about a few days (e.g., 2, 3, 5, 7, 10 or 14 days) or the symptoms of inflammation reoccur at least once (e.g., once or more times, twice or more times or three or more times). In some embodiments, the inflammatory disease or syndrome of the nervous system is characterized by chronic low-grade inflammation. Chronic low-grade inflammation can occur in the absence of clinical symptoms.

In certain embodiments, the subject in need of treatment and/or prevention has a history of a virus infection. Therefore, the subject in need of treatment and/or prevention was infected with the virus at least once. In certain embodiments the subject in need of treatment and/or prevention was infected with the virus at least once. In certain embodiments the subject in need of treatment and/or prevention was infected with the virus at least once during childhood. In certain embodiments the subject in need of treatment and/or prevention was infected with the virus at least once. In certain embodiments the subject in need of treatment and/or prevention was infected with the virus at least once during the last 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 year(s). In certain embodiments, the virus infection is active (e.g. detectable) at the timepoint of diagnosis of the inflammatory disease or syndrome of the nervous system in the subject in need of treatment and/or prevention.

Methods for detecting virus infections are known to the person skilled in the art. In some embodiments, the invention relates to a method for detecting a virus selected from the group of virus isolation, nucleic acid based methods, microscopy based methods, host antibody detection, electron microscopy and host cell phenotype.

In some embodiments, the virus infection described herein is detected in a sample such as in a sample selected from the group of nasopharyngeal swab, blood, tissue (e.g. skin), sputum, gargles, bronchial washings, urine, semen, faeces, cerebrospinal fluid, dried blood spots, nasal mucus.

In some embodiments, the virus infection described herein is obtained as an information retrieved from the patient history.

Examples for detection of a (previous) SARS-CoV-2 infection include Human IFN-γ SARS-CoV-2 ELISpot^{PLUS} kit (ALP), strips (Mabtech, 3420-4AST-P1-1) or determination of a T-cell response (Zuo, J., Dowell, A. C., Pearce, H. et al., 2021, Nat Immunol).

In some embodiments, the inflammatory disease or syndrome of the nervous system described herein is an inflammatory disease or syndrome of the sympathetic nervous system. In some embodiments, the inflammatory disease or syndrome of the nervous system described herein is an inflammatory disease or syndrome of the parasympathetic nervous system. In some embodiments, the inflammatory disease or syndrome of the nervous system described herein is an inflammatory disease or syndrome of the central nervous system. In some embodiments, the inflammatory disease or syndrome of the nervous system described herein is an inflammatory disease or syndrome of the peripheral nervous system.

In certain embodiments, the inflammatory disease or syndrome of the nervous system related to a virus is selected from the group of multiple sclerosis, amyotrophic lateral sclerosis, Alzheimer's disease, Parkinson's disease and Huntington's disease.

Examples of established links of inflammatory disease or syndrome of the nervous system and virus infections:

| | Virus | | |
|---|---|---|---|
| Brain disorder | involved | Virus family | Reference |
| Parkinson's | Hepatitis C | Flaviviridae | Tsai, Liou et al. 2016 |
| Disease (PD) | | | Neurology 86(9): 840-846 |
| | H5N1 | Orthomyxoviridae | Jang, Boltz et al. 2012 J |
| | | | Neurosci 32(5): 1545-1559 |
| | SARS-CoV-2 | Coronaviridae | (Sulzer, Antonini et al. |
| | | | 2020 NPJ Parkinsons Dis |
| | | | 6: 18.) |
| | HIV | Retroviridae | Tse, Cersosimo et al. 2004 |
| | | | Parkinsonism Relat Disord |
| | | | 10(6): 323-334. |
| Alzheimer's | Herpes | Herpesviridae | Abbott 2020 Nature |
| Disease (AD) | | | 587(7832): 22-25. |
| Multiple Sclerosis | Epstein-Barr | Herpesviridae | Soldan and Lieberman, |
| (MS) | | | 2020, Drug Discov Today |
| | | | Dis Models 32(Pt A): 35-52 |
| | Herpes | Herpesviridae | Virtanen and Jacobson, |
| | | | 2012, CNS Neurol Disord |
| | | | Drug Targets 11(5): 528- |
| | | | 544. |

The disease or syndrome is preferably related to a coronavirus infection, and more preferably the disease or syndrome is related to a SARS-CoV-2 infection. The disease of syndrome related to a SARS-CoV-2 disease or syndrome is preferably at least one selected form the group consisting of fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste, lack of appetite, loss of weight, stomach pain, conjunctivits, skin rash, lymphoma, apathy, and somnolence, preferably fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste.

The present invention relates to a composition for use in the treatment and/or prevention of disease or syndrome related to a virus infection, preferably a coronavirus infection in a subject in need thereof, the composition comprising a therapeutically effective amount of an Alpha1-Antitrypsin (AAT) protein, a variant, an isoform and/or a fragment thereof. The coronavirus is preferably SARS-CoV-2.

The inventor(s) found that AAT and rhAAT inhibits viral entry of several viruses and reduces inflammation, in particular in microglia of the nervous system. Furthermore, SH-SY5Y cells that are of neuronal origin and are frequently used to study neurodegenerative disease, including Parkinsons's disease (Xicoy, H., Wieringa, B. & Martens, G. J. The SH-SY5Y cell line in Parkinson's disease research: a systematic review. Mol Neurodegeneration 12, 10, 2017) display a relatively high copy number of spike protein priming proteases mRNA, namely trypsin and cathepsin B. Coupled with a relatively high level of ACE2 expression in SH-SY5Y, these neural tissue (Bielarz V, Willemart K, Avalosse N, et al. Susceptibility of neuroblastoma and glioblastoma cell lines to SARS-CoV-2 infection. Brain Res. 2021 May 1) and cells of similar origin, become susceptible to SARS-CoV-2 viral infection.

Accordingly, the invention is at least in part based on the broad effect of AAT on diseases or syndromes related to virus infections.

Subjects can be particularly suitable to treatment and/or prevention with AAT and/or rhAAT protein.

Therefore, the present invention also relates a composition for use in the treatment and/or prevention of a disease or syndrome related to a virus infection, preferably a coronavirus infection, more preferably a SARS-CoV-2 infection in a subject in need thereof, the composition comprising a therapeutically effective amount of an alpha1-antitrypsin (AAT) protein, a variant, an isoform and/or a fragment thereof, wherein the subject in need thereof has at least one altered level selected from i) endogenous alpha-antitrypsin (AAT), at least one spike protein priming protease, angiotensin converting enzyme 2 (ACE2 receptor) and interferon-gamma (IFN-γ) compared to at least one reference subject. The alpha1-antitrypsin (AAT) protein, a variant, an isoform and/or a fragment thereof can be a plasma-extracted AAT, a variant, an isoform and/or a fragment thereof, in particular a human plasma-extracted AAT, a variant, an isoform and/or a fragment thereof; or a recombinant alpha1-antitrypsin (rhAAT) protein, a variant, an isoform and/or a fragment thereof, preferably the alpha1-antitrypsin (AAT) protein, a variant, an isoform and/or a fragment thereof is a recombinant alpha1-antitrypsin (rhAAT) protein, a variant, an isoform and/or a fragment thereof.

The “subject in need thereof” is also referred to as a subject of interest. The subject in need thereof is a subject can be a subject during or with a virus infection, preferably a coronavirus infection, more preferably a SARS-CoV-2 infection (i.e. an infected subject) having a disease or syndrome related to a virus infection, preferably a coronavirus infection, more preferably a SARS-CoV-2 infection. An infected subject can require treatment and/or prevention of a disease or syndrome related to a virus infection, preferably a coronavirus infection, more preferably a SARS-CoV-2 infection. The treatment with AAT and/or rhAAT can inhibit or reduce the entry of the virus infection, preferably the coronavirus infection, more preferably the SARS-CoV-2 virus into the cells by inhibiting the spike protein priming protease, reduce the propagation of the virus infection, preferably the coronavirus infection, more preferably the SARS-CoV-2 virus in the body and/or reduce inflammation as a response to the virus infection, preferably the coronavirus infection, more preferably the SARS-CoV-2 infection. A subject in need of a treatment and/or prevention can have a respiratory syndrome, more preferably an acute respiratory syndrome, even more preferably a severe acute respiratory syndrome. The subject in need of a treatment and/or prevention may have at least one symptom selected from the group of consisting of fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste, lack of appetite, loss of weight, stomach pain, conjunctivits, skin rash, lymphoma, apathy, and somnolence, preferably from the group consisting of fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste. A subject in need of a treatment and/or prevention may require intensive care and/or artificial ventilation. A subject in need of a treatment and/or prevention can be defined by one of the above definitions or any combination thereof.

A subject in need thereof can also be a subject prior to a virus infection, preferably a coronavirus infection, more preferably a SARS-CoV-2 infection, which is particularly susceptible to develop a disease or syndrome after a virus infection, preferably a coronavirus infection, more preferably a SARS-CoV-2 infection. Such a subject may require prevention of a disease or syndrome related to a virus infection, preferably a coronavirus infection, more preferably a SARS-CoV-2 infection prior to infection.

The at least one reference subject can be a group of reference subjects. Preferably, the reference (reference) subject(s) is/are a subjects with or during a virus infection, preferably a coronavirus infection, more preferably a SARS-CoV-2 infection (i.e. an infected subject) which is/are asymptomatic or has/have mild symptoms, more preferably subject(s) is/are subjects with or during a virus infection, preferably a coronavirus infection, more preferably a SARS-CoV-2 infection, which is/are asymptomatic.

Asymptomatic according to the present invention means that the (reference) subject has no symptoms, preferably has no symptoms selected from the group of consisting of no fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste, lack of appetite, loss of weight, stomach pain, conjunctivits, skin rash, lymphoma, apathy, and somnolence, more preferably has no fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste. An asymptomatic (reference) subject preferably have no respiratory syndrome, more preferably no acute respiratory syndrome, even more preferably no severe acute respiratory syndrome. An asymptomatic (reference) subject does not require intensive care and/or artificial ventilation. An asymptomatic (reference) subject can be defined by one of the above definitions or any combination thereof.

A (reference) subject with mild symptoms according to the present invention preferably have no respiratory syndrome, more preferably no acute respiratory syndrome, even more preferably no severe acute respiratory syndrome. A (reference) subject with mild symptoms preferably does not require intensive care and/or artificial ventilation. A (reference) subject with mild symptoms can have at least one symptom selected from the group consisting of fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste, lack of appetite, loss of weight, stomach pain, conjunctivits, skin rash, lymphoma, apathy, and somnolence, more preferably has no fever, cough, fatigue, difficulty breathing, chills, joint or muscle pain, expectoration, sputum production, dyspnea, myalgia, arthralgia or sore throat, headache, nausea, vomiting, diarrhea, sinus pain, stuffy nose, reduced or altered sense of smell or taste, wherein the (reference) subject with mild symptoms does not require intensive care and/or artificial ventilation. A (reference) subject with mild symptoms can be defined by one of the above definitions or any combination thereof.

A reference subject can be a child, in particular a child having an age of less than 10 years, preferably less than 5 years. A reference subject can have an age of between 1 to 10 years, preferably 2 to 5 years.

A (reference) subject during or with a virus infection, particularly a coronavirus infection, more particularly a SARS-CoV-2 infection is a subject, which is preferably a (reference) subject infected with a virus, particularly a coronavirus, more particularly SARS-CoV-2. An infected (reference) subject means that the virus, particularly the coronavirus, more particularly the SARS-CoV-2 virus has entered the cells of the body of the (reference) subject, in is preferably proliferating in the cells of the body of the (reference) subject.

After infection with a virus, particularly a coronavirus, more particularly SARS-CoV-2, the interferon-gamma levels in the infected subject increase. The increased interferon-gamma levels in turn lead to an increase in the level of the angiotensin converting enzyme 2 (ACE2 receptor). Increased levels of the angiotensin converting enzyme 2 (ACE2 receptor) stimulate increased activity and priming of the spike protein by proteases. Then, the endogenous levels of AAT decrease in response to the increased activity level(s) of at least one spike protein priming protease. Afterwards, endogenous level of AAT depletes further as AAT binds (and inhibits) active spike protein priming proteases. Subjects having at least one selected from the group of i) a lower level of endogenous alpha-antitrypsin (AAT), ii) a higher level of at least one spike protein priming protease, iii) a higher level of angiotensin converting enzyme 2 (ACE2 receptor) and iv) a higher level of interferon-gamma (IFN-γ) compared to at least one reference subject, are particularly susceptible to develop a disease or syndrome in response to the virus, particularly the coronavirus, more particularly the SARS-CoV-2 infection. Therefore, these subjects of interest are particularly relevant and suited to receive treatment and/or prevention using a composition comprising a therapeutically effective amount of an alpha1-antitrypsin (AAT) protein, a variant, an isoform and/or a fragment thereof. The alpha1-antitrypsin (AAT) protein, a variant, an isoform and/or a fragment thereof can be a plasma-extracted AAT, a variant, an isoform and/or a fragment thereof, in particular a human plasma-extracted AAT, a variant, an isoform and/or a fragment thereof; or a recombinant alpha1-antitrypsin (rhAAT) protein, a variant, an isoform and/or a fragment thereof, preferably the alpha1-antitrypsin (AAT) protein, a variant, an isoform and/or a fragment thereof is a recombinant alpha1-antitrypsin (rhAAT) protein, a variant, an isoform and/or a fragment thereof. Most preferably, the AAT protein is recombinant alpha1-antitrypsin (rhAAT) protein, produced in a Chinese Hamster Ovary (CHO) cell and/or in a Human Embryonic Kidney (HEK) cell.

The present invention also relates to a composition for use in the treatment and/or prevention of a disease or syndrome related to a virus infection, preferably a coronavirus infection, more preferably a SARS-CoV-2 infection in a subject in need thereof, the composition comprising a therapeutically effective amount of an alpha1-antitrypsin (AAT) protein and/or recombinant alpha1-antitrypsin (rhAAT) protein, a variant, an isoform and/or a fragment thereof, wherein the subject in need thereof has at least one selected from the group consisting of:
1. a lower level of endogenous alpha-antitrypsin (AAT) prior to a virus infection, preferably a coronavirus infection, more preferably a SARS-CoV-2 infection or during a SARS-CoV-2 infection compared to at least one subject during a virus infection, preferably a coronavirus infection, more preferably a SARS-CoV-2 infection, which is asymptomatic or has mild symptoms,
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
3. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
4. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

The at least one subject, which is asymptomatic or has mild symptoms is also referred to as at least one reference subject. The reference subject is defined as described herein.

The “subject in need thereof” is also referred to as a subject of interest. The in need of a treatment and/or prevention of a disease syndrome related to a virus infection, preferably a coronavirus infection, more preferably a SARS-CoV-2 infection is defined as described herein.

The levels as defined in i) to iv) can be protein levels and/or mRNA levels, preferably a protein or mRNA level. Protein level(s) are measured using antibody-based assays such as enzyme-linked immunosorbent assay (ELISA), and/or bio-layer interferometry (BLI) based on fiber optic biosensors (ForteBio Octet).

Level of spike protein priming protease can be determined by measuring the activity of the spike protein protease using fluorogenic peptides derived from SARS-CoV-2 spike protein. The method is described in Jaimes et. al (Javier A. Jaimes, Jean K. Millet, Gary R. Whittaker Proteolytic Cleavage of the SARS-CoV-2 Spike Protein and the Role of the Novel S1/S2 Site, CELL, iScience 23, 101212, Jun. 26, 2020). Transparent Methods Peptides: Fluorogenic peptides derived from SARS-CoV-2 spike (S) S1/S2 sites composed of the sequences HTVSLLRSTSQ (SEQ ID NO: 3) and TNSPRRARSVA (SEQ ID NO: 4) sequences, respectively, and harboring the (7-methoxycoumarin-4-yl) acetyl/2,4-dinitrophenyl (MCA/DNP) FRET pair were synthesized by Biomatik (Wilmington, DE, USA). Recombinant furin can be purchased from New England Biolabs (Ipswich, MA, USA). Recombinant L-1-Tosylamide-2-phenylethyl chloromethyl ketone (TPCK)-treated trypsin can be obtained from Sigma-Aldrich (St Louis, MO, USA). Recombinant PC1, matriptase, cathepsin B, and cathepsin L can be purchased from R&D Systems (Minneapolis, MN, USA). Fluorogenic peptide assay: For each fluorogenic peptide, a reaction is performed in a 100 μL volume with buffer composed of 100 mM Hepes, 0.5% Triton X-100, 1 mM CaCl2 and 1 mM 2-mercaptoethanol pH 7.5 for furin (diluted to 10 U/mL); 25 mM MES, 5 mM CaCl2, 1% (w/v) Brij-35, pH 6.0 for PC1 (diluted to 2.2 ng/μL); PBS for trypsin (diluted to 8 nM); 50 mM Tris, 50 mM NaCl, 0.01% (v/v) Tween® 20, pH 9.0 for matriptase (diluted to 2.2 ng/μL); 25 mM MES, pH 5.0 for cathepsin B (diluted to 2.2 ng/μL); 50 mM MES, 5 mM DTT, 1 mM EDTA, 0.005% (w/v) Brij-35, pH 6.0 for cathepsin L (diluted to 2.2 ng/μL) and with the peptide diluted to 50 μM. Reactions are performed at 30° C. in triplicates, and fluorescence emission is measured every minute for 45 min using a SpectraMax fluorometer (Molecular Devices, Sunnyvale, CA, USA), with λex 330 nm and λem 390 nm wavelengths setting, enabling tracking of fluorescence intensity over time and calculation of Vmax of reactions. Assays should be performed in triplicates with results representing averages of Vmax from three independent experiments.

Level of IFN-γ protein could be measured using a flow cytometry, particle-based immunoassay. The method can be adopted from Huang et. al., (Huang K J, Su I J, Theron M, et al. An interferon-gamma-related cytokine storm in SARS patients. J Med Virol. 2005; 75(2):185-194. doi:10.1002/jmv.20255) BD Human Th1/Th2 Cytokine or Chemokine Bead Array (CBA) Kit. The BD Human Th1/Th2 Cytokine CBA Kit (BD PharMingen, San Diego, CA) to measure IFN-γ levels by flow cytometry in a particle-based immunoassay. This kit allowed simultaneous measurement of six cytokines from 50 ml of patient serum sample. The limits of detection of these immunoassays are 7.1 pg/ml for IFN-γ.

Preferably, the endogenous level of AAT described herein is a protein level. The level of the at least one spike protein protease described herein is preferably mRNA level. The level of the ACE2 receptor described herein is preferably a mRNA level. The level of IFN-γ described herein is preferably a protein level. The protein and/or mRNA levels can be measured in blood, urine or saliva, preferably in blood, more preferably in blood plasma, most preferably in human blood plasma.

Several factors of the entry of viruses, particularly coronaviruses, more particularly the SARS-CoV-2 virus into cells and propagation in cells are already understood, while others are still subject of investigation. Entry of the SARS-CoV-2 virus is mediated by the spike protein, spike protein priming protease(s) and an ACE2 receptor. The SARS-CoV-2 spike protein is also referred to as spike protein S. The spike protein priming protease cleaves the spike protein of SARS-CoV-2 thereby priming the SARS-CoV-2 for entry into the cell. SARS-CoV-2 enters the cell by interaction of the primed spike protein with the ACE receptor. Thus, if at least one or more priming protease(s) is present in the subject of interest, the easier and faster the entry of the SARS-CoV-2 virus into the cells. Also, the more ACE2 receptor(s) is present, the easier and faster the entry of the SARS-CoV-2 into the cells. The infection with SARS-CoV-2 leads to inflammation and thus elevated IFN-γ expression. IFN-γ expression in response to a SARS-CoV-2 infection can in turn lead to an increased expression of the ACE2 receptor. During proliferation of SARS-CoV-2, IFN-γ levels increase further, stimulating the increase in ACE2 interaction with the spike protein and subsequently the priming of the spike protein, AAT levels decrease and upon viral entry into the cells, inflammation increases, which leads to even higher levels of IFN-γ. After infection with SARS-CoV-2, first, the IFN-γ levels increase, second, the AAT levels decrease and the levels of cathepsin L increase and/or other spike protein priming (S-priming) proteases increase.

Therefore, the invention is at least in part based on the discovery, that AAT as well as rhAAT simultaneously reduces viral entry and virus associated inflammation, in particularly virus associated inflammation due to high IFN-γ levels. This combined effect is particularly useful in the patient populations described herein.

In certain embodiments the invention relates to the composition for use according to the invention, wherein the spike protein priming protease is at least one selected from the group consisting of transmembrane protease serine subtype 2 (TMPRSS2), transmembrane protease subtype 6 (TMPRSS6), cathepsin L, cathepsin B, proprotein convertase 1 (PC1), trypsin, elastase, neutrophil elastase, matriptase and furin.

In certain embodiments the invention relates to the composition for use according to the invention, wherein the spike protein priming protease is cathepsin L and/or furin.

AAT is endogenously expressed in the human body. AAT is also referred to as alpha-1-proteinase inhibitor. AAT is capable of inhibiting proteases, specifically spike protein proteases, such as transmembrane protease serine subtype 2 (TMPRSS2), transmembrane protease subtype 6 (TMPRSS6/matriptase-2), cathepsin L, cathepsin B, proprotein convertase 1 (PC1), trypsin, elastase, neutrophil elastase, matriptase and furin. If the levels of the four players of the present invention (AAT, spike protein priming protease(s), ACE2 receptor and IFN-γ) are altered in an infected subject of interest compared to a reference subject, the infected subject of interest may benefit particularly form a treatment and/or prevention of a diseases or syndrome related with a SARS-CoV-2 infection. Low endogenous AAT levels can lead to a higher susceptibility of a subject of interest to develop a disease or syndrome related to SARS-CoV-2, in particular to develop COVID-19.

In certain embodiments the invention relates to the composition for use according to the invention, wherein the lower level of endogenous AAT prior to a virus infection or during a virus infection is caused by AAT-deficiency.

Alpha1-Antitrypsin (hereafter “AAT”) is a protein that naturally occurs in the human body and is produced in the liver, preferably in hepatocytes. According to Janciauskiene et. al., (Janciauskiene S M, Bals R, Koczulla R, Vogelmeier C, Köhnlein T, Welte T. The discovery of a1-antitrypsin and its role in health and disease. Respir Med. 2011; 105(8):1129-1139. doi:10.1016/j.rmed.2011.02.002) the normal plasma concentration of AAT ranges from 0.9 to 1.75 g/L. Considering a MW of 52,000 (Brantly M, Nukiwa T, Crystal RG. Molecular basis of alpha-1-antitrypsin deficiency. Am J Med. 1988; 84(6A):13-31. doi:10.1016/0002-9343(88)90154-4), this corresponds to 16 to 32 μM normal blood plasma concentrations. Crystal 1990 (Crystal RG. Alpha 1-antitrypsin deficiency, emphysema, and liver disease. Genetic basis and strategies for therapy. J Clin Invest. 1990 May; 85(5):1343-52. doi: 10.1172/JC1114578. PMID: 2185272; PMCID: PMC296579) describes that 11 μM is the threshold level for the clinical manifestation of AAT-deficiency. For most healthy individuals, 2 g daily expression of AAT in the liver is enough to reach this critical serum level of 11 μM, the endogenous AAT level is then sufficient to protect the lower respiratory tract from destruction by neutrophil elastase (NE) and inhibiting the progressive destruction of the alveoli, which culminates in emphysema. Crystal 1990 further notes that normal endogenous levels of AAT in healthy individuals vary between 20-53 μM. Endogenous levels of AAT protein in the blood plasma of healthy human subjects prior to a virus infection, preferably a coronavirus infection, more preferably a SARS-CoV-2 infection range between 5 and 60 μM, preferably between 10 and 40 μM, more preferably between 25 to 30 μM, even more preferably between 16 and 32 μM. Alternatively the Endogenous levels of AAT protein in the blood plasma of healthy human subjects prior to a virus infection, preferably a coronavirus infection, more preferably a SARS-CoV-2 infection is preferably higher than 30 μM, more preferably higher than 40 μM, most preferably higher than 50 μM. AAT protein can be plasma AAT protein or recombinant AAT (rhAAT) protein. In some embodiments, Plasma AAT protein is derived from blood plasma. In some embodiments, recombinant AAT protein is produced recombinantly, for example in HEK cells, CHO cells or E. coli cells. Pharmaceutical companies worldwide derive AAT protein from human blood plasma for the treatment of AAT-deficiency, a hereditary disorder. Plasma derived AAT is approved in the US and the EU, by the Food and Drug Administration (FDA) and the European Medicines Agency (EMA), respectively. Preferably, the AAT protein of the invention has the human amino acid sequence, most preferably as set forth in SEQ ID NO: 1.

10 μM of AAT reduces pseudoviral entry by 20-30% in A549 cells, which overexpress the ACE2 receptor.

According to Azouz et. al. (Nurit P. Azouz, Andrea M. Klingler and Marc E. Rothenberg, Alpha1-Antitrypsin (AAT) is an Inhibitor of the SARS-CoV-2-Priming Protease TMPRSS2, (bioRxiv preprint online, https://doi.org/10.1101/2020.05.04.077826, posted on May 5, 2020) concentrations of 1-100 μM AAT achieve dose-dependent inhibition of TMPRSS2 proteolytic activity.

100 μM of AAT reduces pseudoviral entry by 50-75% in A549 cells, which overexpress the ACE2 receptor only. 100 μM of AAT reduces pseudoviral entry by up to 45% only in A549 cells that overexpress both the ACE2 receptor and the spike protein priming protease TMPRSS2.

It is important to note that viral entry is observed independently of TMPRSS2. Demonstrating that priming proteases such as furin and/or cathepsin L are able to replace TMPRSS2, probably among others. In this regard, AAT as well as rhAAT reduces the activity of the proteases cathepsin B, cathepsin L, trypsin, furin, PC1, Matriptase, elastase and neutrophil elastase.

Therefore, the inhibitory effect of AAT as well as rhAAT on viral entry extends beyond the effect of other TMPRSS2 inhibitors, in that AAT as well as rhAAT effectively inhibits several priming proteases (e.g., proteases that can replace TMPRSS2 function) and subsequent ACE2 mediated viral entry.

Therefore, AAT as well as rhAAT reduces viral entry by reducing priming protease activity, in particular by broadly and efficiently reducing priming protease(s) activity.

Accordingly, the invention is at least in part based on the surprising finding that compositions comprising AAT and/or rhAAT, variants, isoforms and/or fragments thereof, are particularly effective in the treatment and/or prevention of a disease or syndrome related to a virus infection, preferably a coronavirus infection, more preferably a SARS-CoV-2 infection, in particular in a subject with one or more of the preconditions described herein.

In the present invention, the lower level of endogenous AAT according to i) is preferably a protein level in human blood plasma. The level of endogenous AAT protein in human blood plasma according to i) is preferably less than 200 μM, preferably less than 150 μM, 100 μM, less than 90, μM, less than 80 μM, less than 70 μM, less than 60 μM, less than 50 μM, less than 40 μM, less than 30 μM, less than 25 μM, less than 20 μM, less than 15 μM, less than 11 μM or less than 10 μM. More preferably less than 200 μM, less than 100 μM, less than 25 μM, less than 15 μM, or less than 11 μM. A low level of AAT is not sufficient to successfully inhibit the spike protein priming protease(s). A low level of endogenous AAT can therefore promote virus proliferation, in particular coronavirus proliferation, more particularly SARS-CoV-2 proliferation and/or the development of a disease of syndrome related to a virus infection, preferably a coronavirus infection, more preferably a SARS-CoV-2 infection. A lower level of endogenous AAT prior to a virus infection, preferably a coronavirus infection, more preferably a SARS-CoV-2 infection or during a virus infection, preferably a coronavirus infection, more preferably a SARS-CoV-2 infection can be caused by AAT deficiency, preferably lower level of endogenous AAT prior to a virus infection, preferably a coronavirus infection, more preferably a SARS-CoV-2 infection is caused by AAT deficiency. The AAT-deficiency is a condition that is inherited in an autosomal codominant pattern. Codominance means that two different versions of the gene may be active (expressed), and both versions contribute to the genetic trait. The most common version (allele) of the SERPINA1 gene, called M, produces normal levels of alpha-1 antitrypsin. Most people in the general population have two copies of the M allele (MM) in each cell. Other versions of the SERPINA1 gene lead to reduced levels of alpha-1 antitrypsin. For example, the S allele produces moderately low levels of this protein, and the Z allele produces very little alpha-1 antitrypsin. Individuals with two copies of the Z allele (ZZ) in each cell are likely to have alpha-1 antitrypsin deficiency. Those with the SZ combination have an increased risk of developing lung diseases (such as emphysema), particularly if they smoke. Worldwide, it is estimated that 161 million people have one copy of the S or Z allele and one copy of the M allele in each cell (MS or MZ). Individuals with an MS (or SS) combination usually produce enough alpha-1 antitrypsin to protect the lungs. People with MZ alleles, however, have a slightly increased risk of impaired lung or liver function. Subjects with an AAT deficiency in the present invention preferably have ZZ mutation, SZ mutation, MS mutation, MZ mutation or SS mutation of the SERPINA1 gene, preferably a ZZ mutation. The low levels of AAT secretion in the ZZ mutation of the SERPINA1 gene are due to misfolding of AAT and its subsequent accumulation in the endoplasmic reticulum (ER) of hepatocytes (Crystal 1990), resulting in progressive liver disease as the accumulation of misfolded AAT negatively affects hepatocytes' health leading to their ultimate demise.

A lower level of endogenous AAT during a virus infection, preferably a coronavirus infection, more preferably a SARS-CoV-2 infection can also be caused by the virus infection, the coronavirus infection, or the SARS-CoV-2 infection respectively. That is, the level of endogenous AAT in a reference subject can increase temporarily during the virus infection as healthy hepatocytes attempt to overcompensate for the drop in endogenous AAT levels, while the level of endogenous AAT in a subject with genetic AAT-deficiency is lower due to absence or incomplete virus infection-induced increase (lack of healthy hepatocytes).

A lower level of endogenous AAT may also be caused by a liver disease such as a non-alcoholic fatty liver disease, diabetes mellitus type 1 or 2 (preferably type 1), obesity and cardiovascular conditions. Fatty acid deposit build-up in the liver leads to increased stress (increased IFN-γ), tissue inflammation and subsequent damage to hepatocytes causing lower level of healthy secretion of AAT. As with other types of liver maladies, it is important to note that AAT-deficiency leads to liver disease over time as the accumulation of misfolded AAT in the ER of hepatocytes ultimately causes liver failure.

In the present invention, the spike protein priming protease can be any protease capable of priming the spike protein of a virus, preferably a coronavirus, more preferably SARS-CoV-2. Preferably, the spike protein priming protease is at least one selected from the group consisting of transmembrane protease serine subtype 2 (TMPRSS2), transmembrane protease subtype 6 (TMPRSS6), cathepsin L, cathepsin B, proprotein convertase 1 (PC1), trypsin, elastase, neutrophil elastase, matriptase and furin, more preferably TMPRSS2, cathepsin L and furin, even more preferably cathepsin L or furin. The spike protein priming protease is furin is also referred to as paired basic amino acid cleaving (PACE) enzyme.

The higher level of the at least one spike protein priming protease, preferably Cathepsin L, described herein is preferably a mRNA level in human blood plasma, or a protein level in human blood plasma. The plasma concentration of Cathepsin L in healthy subjects is 0.2 to 1 ng/mL (i.e. 10 to 50 μM given a molecular weight of about 23-24 kDa; (Kirschke 1977 https://febs.onlinelibrary.wiley.com/doi/10.1111/j.1432-1033.1977.tb11393.x). Moreover, immune cells are known to be a major source of extracellular cysteine cathepsins in inflammation, including in the brain (Hayashi et al., 2013, von Bernhardi et al., 2015, Wendt et al., 2008, Wendt et al., 2007). The level of the at least one spike protein priming protease protein described herein is preferably higher than 0.2, 0.5 or 1 ng/ml in human blood plasma. The level of the at least one spike protein priming protein described herein protease is preferably higher than 10, 20, 30, 40, 50, 75 or 100 μM, preferably higher than 10 or 50 μM, even more preferably higher than 50 μM. In this embodiment the at least one spike protein priming protease(s) is preferably cathepsin L. In certain embodiments, the at least one spike protein priming protease(s) described herein are at least two, at least three, at least four, at least five, at least six, at least seven, at least eight, at least nine spike protein priming proteases. In certain embodiments, the at least one spike protein priming protease(s) described herein is at least one protease selected from the group consisting of TMPRSS2, cathepsin B, cathepsin L, trypsin, furin, PC1, matriptase, elastase and neutrophil elastase. In certain embodiments, the at least one spike protein priming protease(s) described herein is at least one protease selected from the group consisting of TMPRSS2, cathepsin B, cathepsin L, trypsin, furin, PC1 and matriptase.

In certain embodiments the invention relates to the composition for use according to the invention, wherein the higher level of at least one spike protein priming protease is caused by age and/or a genetic predisposition.

The higher level of the at least one spike protein priming protease can be caused by age and/or a genetic predisposition. In particular, higher level of cathepsin B and L, preferably of cathepsin B can be caused by age. Higher levels of spike protease protein, in particular cathepsin B and L, more preferably cathepsin B can accumulate in the lysosome. The level of spike protein priming protease is higher in subjects of interest, with an age of more than 50 years, preferably more than 60 years, more preferably more than 70 years, even more preferably more than 80 years, and most preferably more than 90 years. Subjects of Afro-American origin may have a genetic predisposition for higher levels of spike protein priming protease, in particular furin. HeLa cells differ in relative mRNA rates of furin and Cathepsin L compared to A549 cells. HeLa cells are of Afro-American origin. A549 cells are airway epithelial cells of Caucasian origin. HeLa cells are more susceptible to SARS-CoV-2 entry than A549 cells and are less reactive to treatment with AAT. A genetic predisposition can be determined by genetic profiling of individual subjects of interest.

In certain embodiments the invention relates to the composition for use according to the invention, wherein the higher level of ACE2 receptor is caused by at least one selected from the group of an infection, inflammation, age and a genetic predisposition.

The higher level of ACE 2 receptor described herein is preferably an mRNA level in human blood plasma. The higher level of the ACE2 receptor can be caused by at least one selected from the group of an infection, inflammation (e.g. IFN-γ), age and a genetic predisposition. The infection can be a viral infection and/or a bacterial infection, preferably a viral infection, more preferably a coronavirus infection, even more preferably a SARS or SARS-CoV-2 infection. A further example for infection is Leishmaniasis. ACE2 receptor expression is upregulated in response to higher levels of IFN-γ, which levels in turn increase as an immune response to inflammation. The inflammation can be caused, for example, by a bacterial and/or viral infection, cancer, delayed type hypersensitivity; autoimmune diseases (such as autoimmune encephalomyelitis, rheumatoid arthritis, autoimmune insulitis (also referred to as type 1 diabetes mellitus), allograft rejection and graft versus host reaction, nonspecific inflammation and cytokine release. The age is preferably an age of more than 50 years, preferably more than 60 years, more preferably more than 70 years, even more preferably more than 80 years, and most preferably more than 90 years. An example of a genetic predisposition for high levels of IFN-γ is familial Mediterranean fever. A genetic predisposition can be determined by genetic profiling of individual subjects of interest. A higher level of ACE2 receptor described herein can also be present in a tissue selected from the group consisting of lung (Calu3), colon (CaCo2), liver (HEPG2), kidney (HEK-293T), and the brain (SH-SY5Y) as confirmed by qPCR.

In certain embodiments the invention relates to the composition for use according to the invention, wherein the higher level of IFN-γ is caused by at least one selected from the group of an infection, inflammation, age and a genetic predisposition.

The higher level of interferon-gamma (IFN-γ) according to iv) is preferably a protein or mRNA level in human blood plasma, more preferably a protein level in human blood plasma. In healthy humans, the IFN-γ level is below or around the limit of detection of the assays, e.g. less than 30 to 50 pg/mL) (Billau 1996; Kimura 2001). IFN-γ is produced almost exclusively by natural killer (NK) cells, CD4+ and some CD8+ lymphocytes. Production of IFN-γ by either NK or T cells requires cooperation of accessory cells, mostly mononuclear phagocytes, which also need to be in some state of activation (Billiau A. Interferon-gamma: biology and role in pathogenesis. Adv Immunol. 1996; 62:61-130. doi:10.1016/s0065-2776(08)60428-9). Thus, inflammatory conditions leading to NK cells activation and T cells activation lead to increased IFN-γ levels. Inflammation state involving circulating NK or T cells (infection, cancer) are expected to lead to higher plasma levels.

The following Table provides values for plasma levels of IFN-γ in several conditions.

TABLE 1

| | | | IFN-γ- | |
|---|---|---|---|---|
| | Type of | IFN-γ | Control | |
| Condition | condition | (pg/mL) | (pg/mL) | Reference |
| SARS | infection | 456 | 3.3 | Huang et. al., |
| | | (acute | (control) | Huang, K.-J., Su, I.-J., Theron, |
| | | phase) | 0.19 pM | M., Wu, Y.-C., Lai, S.-K., Liu, |
| | | 26.6 pM | | C.-C. and Lei, H.-Y. (2005), An |
| | | | | interferon-γ-related cytokine |
| | | | | storm in SARS patients. J. Med. |
| | | | | Virol., 75: 185-194. |
| Leishmaniasis | infection | 118 | 31 | Hailu et. al., |
| | | (untreated) | (treated) | Hailu A, van der Poll T, Berhe |
| | | 6.9 pM | 1.8 pM | N, Kager PA. Elevated plasma |
| | | | | levels of interferon (IFN)- |
| | | | | gamma, IFN-γ inducing |
| | | | | cytokines, and IFN-γ inducible |
| | | | | CXC chemokines in visceral |
| | | | | leishmaniasis. Am J Trop Med |
| | | | | Hyg. 2004; 71(5): 561-567. |
| Familial | Genetic | 19.4 | 4.8 | Köklü et. al., |
| Mediterranean | disorder | (acute | (attack- | Köklü S, Oztürk MA, Balci M, |
| fever | | attack) | free) | Yüksel O, Ertenli I, Kiraz S. |
| | | 1.1 pM | 0.28 pM | Interferon-gamma levels in |
| | | | | familial Mediterranean |
| | | | | fever. Joint Bone Spine. |
| | | | | 2005; 72(1): 38-40. |
| | | | | doi: 10.1016/j.jbspin.2004.03.011 |

The level of IFN-γ protein in human blood plasma according to iv is preferably high than 1, 5, 10, 15, 20, 25, 30, 40, 50, 75, 100, 125, 150, 200, 300, 400 or 500 pg/ml. The higher level of IFN-γ is preferably caused by at least one selected from the group of an infection, inflammation, age and a genetic predisposition, more preferably by inflammation. The infection can be a viral infection and/or a bacterial infection, preferably a viral infection, more preferably a coronavirus infection, even more preferably a SARS or SARS-CoV-2 infection. A further example for infection is Leishmaniasis. The inflammation can be caused, for example, by a bacterial and/or viral infection, cancer, delayed type hypersensitivity; autoimmune diseases (such as autoimmune encephalomyelitis, rheumatoid arthritis, autoimmune insulitis (also referred to as type 1 diabetes mellitus), allograft rejection and graft versus host reaction, nonspecific inflammation and cytokine release. The age is preferably an age of more than 50 years, preferably more than 60 years, more preferably more than 70 years, even more preferably more than 80 years, and most preferably more than 90 years. An example of a genetic predisposition for high levels of IFN-γ is familial Mediterranean fever. A genetic predisposition can be determined by genetic profiling of individual subjects of interest.

Accordingly, the invention is at least in part based on the surprising finding that compositions comprising AAT as well as rhAAT, variants, isoforms and/or fragments thereof, reduce both virus proliferation and inflammation, in particular IFN-γ associated inflammation.

In one embodiment, “subject in need thereof”, i.e. the subject of interest has two selected from the group consisting of:
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
3. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
4. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

Thus, the “subject in need thereof” can have
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

Preferably, in this embodiment, the AAT protein level in human blood plasma described herein is lower than 52 μM and the cathepsin L protein level in human blood plasma is higher than 10 μM.

In another embodiment, the “subject in need thereof” can have
1. a lower level of endogenous alpha-antitrypsin (AAT) prior to a virus infection, preferably a coronavirus infection, more preferably a SARS-CoV-2 infection or during a SARS-CoV-2 infection compared to at least one subject during a virus infection, preferably a coronavirus infection, more preferably a SARS-CoV-2 infection, which is asymptomatic or has mild symptoms, and
2. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In yet a further embodiment, the “subject in need thereof” can have
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
2. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In these embodiments, 1. and 4. can be at least one disease or condition selected from the group consisting of AAT-deficiency, a liver disease such as a non-alcoholic fatty liver disease, diabetes, obesity and a cardiovascular condition. In some embodiments the invention relates to the composition for use according to the invention, wherein the higher level of IFN-γ is caused by at least one disease or condition selected from the group consisting of AAT-deficiency, a liver disease such as a non-alcoholic fatty liver disease, diabetes, obesity and a cardiovascular condition. In some embodiments the invention relates to the composition for use according to the invention, wherein the lower level of AAT is caused by at least one disease or condition selected from the group consisting of AAT-deficiency, a liver disease such as a non-alcoholic fatty liver disease, diabetes, obesity and a cardiovascular condition. Diabetes can be diabetes mellitus type 1 or type 2. Liver disease can be acetaminophen-induced liver injury, severe chronic hepatitis, alcoholic liver disease (ALD), encompassing a broad spectrum of phenotypes including simple steatosis, steatohepatitis, liver fibrosis and cirrhosis or even HCC (hepatocellular carcinoma). Cardiovascular condition can be any condition brought on by a sudden reduction or blockage of blood flow to the heart, cardiac infraction, acute coronary syndrome (ACS), also in patients with acute myocardial infarction, whereby the left ventricular ejection fraction is inversely correlated with AAT concentrations in the serum, suggesting that systolic dysfunction is associated with an inflammatory response.

Preferably, in this embodiment, the AAT protein level in human blood plasma described herein is lower than 52 μM and the IFN-γ protein level in human blood plasma described herein is higher than 0.19 μM.

In a further embodiment, “subject in need thereof”, i.e. the subject of interest has two selected from the group consisting of:
1. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
2. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
3. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In yet a further embodiment, the “subject in need thereof” can have
1. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
2. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In yet a further embodiment, the “subject in need thereof” can have
1. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
2. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In yet a further embodiment, the “subject in need thereof” can have
1. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
2. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In a further embodiment, the “subject in need thereof”, i.e. the subject of interest has three selected from the group consisting of:
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
3. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
4. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

Thus, the “subject in need thereof” can have
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
3. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In a further embodiment, the “subject in need thereof” can have
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
3. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

Preferably, in this embodiment, the AAT protein level in human blood plasma described herein is lower than 36 μM, the cathepsin L protein level in human blood plasma described herein is higher than 10 μM, the IFN-γ protein level in human blood plasma described herein is higher than 0.19 mM.

More preferably, in this embodiment, the AAT protein level in human blood plasma described herein is lower than 52 μM, the cathepsin L protein level in human blood plasma described herein is higher than 10 μM, the IFN-γ protein level in human blood plasma described herein is higher than 0.19 mM.

In a further embodiment, the “subject in need thereof” can have
1. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
2. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
3. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

In a further embodiment, the “subject in need thereof”, i.e. the subject of interest has
1. a lower level of endogenous alpha-antitrypsin (AAT) prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms
2. a higher level of at least one spike protein priming protease prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms,
3. a higher level of angiotensin converting enzyme 2 (ACE2 receptor) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms, and
4. a higher level of interferon-gamma (IFN-γ) in a subject prior to a virus infection, preferably a coronavirus infection, more preferably a SARS-CoV-2 infection or during a virus infection, preferably a coronavirus infection, more preferably a SARS-CoV-2 infection compared to at least one subject during a virus infection, preferably a coronavirus infection, more preferably a SARS-CoV-2 infection, which is asymptomatic or has mild symptoms.

AAT protein in the composition for use of the present invention can plasma AAT protein, a variant, an isoform and/or a fragment thereof; or recombinant AAT protein, a variant, an isoform and/or a fragment thereof. Preferably, the AAT protein, a variant, an isoform and/or a fragment thereof is recombinant AAT protein, a variant, an isoform and/or a fragment thereof. Plasma AAT protein is preferably derived from blood plasma AAT protein, more preferably from human blood plasma (also referred to as human plasma-extracted AAT). In certain embodiments, the invention relates to the composition for use according to the invention, wherein the alpha1-antitrypsin (AAT) protein, a variant, an isoform and/or a fragment thereof is recombinant alpha1-antitrypsin (also referred to as rhAAT), a variant, an isoform and/or a fragment thereof.

Recombinant AAT protein is produced recombinantly, for example in CHO cells, HEK cells (HEK293 and/or HEK293T) or E. coli cells. Pharmaceutical companies worldwide derive AAT protein from human blood plasma for the treatment of AAT-deficiency, a hereditary disorder. Plasma derived AAT is FDA and EMA approved. Preferably, the AAT protein of the invention has the human amino acid sequence, most preferably as set forth in SEQ ID NO: 1. Recombinant AAT protein a variant, an isoform and/or a fragment thereof is preferably free from an Fc-domain and/or histidine-tag (His-tag).

The inventor(s) found that recombinant AAT (rhAAT produced in CHO) binds to an AAT-Antibody with a different affinity and has a more pronounced biologic effect than a plasma-derived AAT. Particularly, recombinant AAT (rhAAT) inhibits ACE2/Spike protein mediated cell fusion more effectively than plasma derived AAT and has a different enzymatic inhibition profile.

Accordingly, the invention is at least in part based on the surprising finding that recombinant AAT (rhAAT) produced in CHO cells is particularly effective for use in the treatment and/or prevention of a disease or syndrome related to a virus infection, preferably a coronavirus infection, more preferably a SARS-CoV-2 infection.

In certain embodiments, the invention relates to the composition for use according to the invention, wherein the alpha1-antitrypsin (AAT) protein, a variant, an isoform and/or a fragment thereof is recombinant alpha1-antitrypsin produced by in human cells (e.g. HEK293 or HEK293T cells).

The inventor(s) found that recombinant AAT produced in human cells, specifically in HEK293 (rhAAT without a His-tag), is particular effective in the reduction of enzymatic activity of Cathepsin L, Trypsin, Furin and Neutrophil Elastase compared to recombinant AAT (rhAAT) produced in CHO cells as well as plasma-derived AAT.

Accordingly, the invention is at least in part based on the surprising finding that recombinant AAT produced in human cells, specifically in HEK293, is particularly effective for use in the treatment and/or prevention of a disease or syndrome related to a virus infection, preferably a coronavirus infection, more preferably a SARS-CoV-2 infection.

In certain embodiments, the invention relates to the composition for use according to the invention, wherein the alpha1-antitrypsin (AAT) protein, a variant, an isoform and/or a fragment thereof is recombinant alpha1-antitrypsin (rhAAT produced in CHO) having more non-human glycan profile. In certain embodiments, the non-human glycan profile described herein is a mammalian-cell-derived glycan profile and/or a glycoengineered glycan profile. Glycoengineering strategies, e.g., used to reduce fucosylation and/or enhance sialylation of glycoproteins are known to the person skilled in the art. In certain embodiments, the non-human glycan profile described herein is a CHO-cell-derived glycan profile. CHO cells express a different glycosylation machinery than human cells, which results in different composition of glycans at the surface of recombinant proteins (Lalonde, M. E., & Durocher, Y., 2017, Journal of biotechnology, 251, 128-140).

In certain embodiments, the invention relates to the composition for use according to the invention, wherein the AAT protein, a variant, an isoform and/or a fragment thereof is recombinant AAT produced by pXC-17.4 (GS System, Lonza), a variant, an isoform and/or a fragment thereof.

The inventor(s) found that the recombinant AAT produced by pXC-17.4 (GS System, Lonza) in CHO (Recombinant AAT 1) induces a more pronounced inhibitory effect on the activity of Elastase and Neutrophil Elastase than plasma-derived alpha1-proteinase inhibitor (Plasma-derived AAT) and recombinant AAT produced by PL136/PL137 (pCGS3, Merck) in CHO (Recombinant AAT 2).

Accordingly, the invention is at least in part based on the surprising finding, that certain forms of recombinant AAT (rhAAT) are particularly effective for use in the treatment and/or prevention of a disease or syndrome related to a virus infection, preferably a coronavirus infection, more preferably a SARS-CoV-2 infection.

As used herein, a “fragment” of an AAT protein, peptide or polypeptide of the invention refers to a sequence containing less amino acids in length than the AAT protein, peptide or polypeptide of the invention, in particular less amino acids than the sequence of AAT as set forth in SEQ ID NO:1. The fragment is preferably a functional fragment, e.g. a fragment with the same biological activities as the AAT protein as set forth in SEQ ID NO:1. The functional fragment preferably derived from the AAT protein as set forth in SEQ ID NO:1. Any AAT fragment can be used as long as it exhibits the same properties, i.e. is biologically active, as the native AAT sequence from which it derives.

The functional AAT fragment can comprise or consist of a C-terminal fragment of AAT as set forth in SEQ ID NO: 2. The C-terminal fragment of SEQ ID NO: 2 consist of amino acids 374 to 418 of SEQ ID NO: 1.

More preferably, the AAT fragment is a fragment containing less amino acids in length than the C-terminal AAT sequence 374-418 (SEQ ID NO: 2). Alternatively, the AAT fragment consists essentially in SEQ ID NO: 2.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80% or at least about 85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

In some embodiments, the invention relates to the composition for use according to the invention, wherein the alpha1-antitrypsin fragment is a C-terminal sequence fragment, or any combination thereof.

The peptidic variants may be linear peptides or cyclic peptides and may be selected from the group comprising short cyclic peptides derived from the C-terminal sequence as set forth in SEQ ID No. 2. Preferably, the short cyclic peptides derived from the C-terminal sequence of Alpha1-Antitrypsin will be selected from the non-limiting group comprising Cyclo-(CPFVFLM)-SH, Cyclo-(CPFVFLE)-SH, Cyclo-(CPFVFLR)-SH, and Cyclo-(CPEVFLM)-SH, or any combination thereof.

As used herein, an “isoform” of an AAT protein, peptide or polypeptide of the invention refers to a splice variant resulting from alternative splicing of the AAT mRNA.

In some embodiments, the amino acid sequence of AAT, the variant, isoform or fragment thereof, as described herein, is at least 80% identical to the corresponding amino acid sequence in SEQ ID NO: 1. In some embodiments, the amino acid sequence of AAT, the variant, isoform or fragment thereof is 80%, 81%, 82%, 83%, 84%, 85%, 86%, 87%, 88%, 89%, 90%, 91%, 92%, 93%, 94%, 95%, 96%, 97%, 98%, or 99% identical to a corresponding amino acid sequence in SEQ ID NO: 1.

The peptide, isoform, fragment or variant thereof of the invention may preferably be conjugated to an agent that increases the accumulation of the peptide, isoform, fragment or variant thereof in the target cell, preferably a cell of the respiratory tract. Such an agent can be a compound which induces receptor mediated endocytosis such as for example the membrane transferrin receptor mediated endocytosis of transferrin conjugated to therapeutic drugs (Qian Z. M. et al., “Targeted drug delivery via the transferrin receptor-mediated endocytosis pathway” Pharmacological Reviews, 54, 561, 2002) or a cell membrane permeable carrier which can, be selected e. g. among the group of fatty acids such as decanoic acid, myristic acid and stearic acid, which have already been used for intracellular delivery of peptide inhibitors of protein kinase C (Ioannides C. G. et al., “Inhibition of IL-2 receptor induction and IL-2 production in the human leukemic cell line Jurkat by a novel peptide inhibitor of protein kinase C” Cell Immunol., 131, 242, 1990) and protein-tyrosine phosphatase (Kole H. K. et al., “A peptide-based protein-tyrosine phosphatase inhibitor specifically enhances insulin receptor function in intact cells” J. Biol. Chem. 271, 14302, 1996) or among peptides. Preferably, cell membrane permeable carriers are used. More preferably a cell membrane permeable carrier peptide is used.

In case the cell membrane permeable carrier is a peptide then it will preferably be a positively charged amino acid rich peptide.

Preferably such positively charged amino acid rich peptide is an arginine rich peptide. It has been shown in Futaki et al. (Futaki S. et al., “Arginine-rich peptides. An abundant source of membrane-permeable peptides having potential as carriers for intracellular protein delivery” J. Biol. Chem., 276, 5836, 2001), that the number of arginine residues in a cell membrane permeable carrier peptide has a significant influence on the method of internalization and that there seems to be an optimal number of arginine residues for the internalization, preferably they contain more than 6 arginines, more preferably they contain 9 arginines. An arginine rich peptide comprises preferably at least 6 arginines, more preferably at least 9 arginines.

The peptide, isoform, fragment or variant thereof may be conjugated to the cell membrane permeable carrier by a spacer (e.g. two glycine residues). In this case, the cell membrane permeable carrier is preferably a peptide.

Usually arginine rich peptides are selected from the non-limiting group comprising the HIV-TAT 48-57 peptide (GRKKRRQRRR; SEQ ID NO. 5), the FHV-coat 35-49 peptide (RRRRNRTRRNRRRVR; SEQ ID NO. 6), the HTLV-II Rex 4-16 peptide (TRRQRTRRARRNR; SEQ ID NO. 7) and the BMV gag 7-25 peptide (SEQ ID NO. 8). Any cell membrane permeable carrier can be used as determined by the skilled artisan.

Since an inherent problem with native peptides (in L-form) is degradation by natural proteases, the peptide, isoform, fragment or variant thereof as well as the cell membrane permeable peptide, of the invention may be prepared to include D-forms and/or “retro-inverso isomers” of the peptide. In this case, retro-inverso isomers of fragments and variants of the peptide, as well as of the cell membrane permeable peptide, of the invention are prepared.

The peptide, isoform, fragment or variant thereof of the invention, optionally conjugated to an agent which increases the accumulation of the peptide in a cell can be prepared by a variety of methods and techniques known in the art such as for example chemical synthesis or recombinant techniques as described in Maniatis et al. 1982, Molecular Cloning, A laboratory Manual, Cold Spring Harbor Laboratory.

The peptide, isoform, fragment or variant thereof of the invention, optionally conjugated to an agent which increases the accumulation of the peptide in a cell as described herein are preferably produced, recombinantly, in a cell expression system. A wide variety of unicellular host cells are useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, RI. 1, B-W and L-M cells, African Green Monkey kidney cells (e. g., COS 1, COS 7, BSCI, BSC40, and BMTIO), insect cells (e. g., Sf9), and human cells and plant cells in tissue culture.

By “therapeutically effective dose or amount” of an Alpha1-Antitrypsin protein, a variant, an isoform and/or a fragment thereof of the invention is intended an amount that when administered brings about a positive therapeutic or prophylactic response with respect to treatment of a subject for a disease or syndrome related to a coronavirus infection.

The term “coronavirus infection” can refer to an infection caused by a coronavirus selected from group comprising MERS-CoV, SARS-CoV and SARS-CoV-2 as well as any variant thereof. In some embodiments, the SARS-CoV-2 variant described herein is a SARS-CoV-2 variant selected from the group of Lineage B.1.1.207, Lineage B.1.1.7, Cluster 5, 501.V2 variant, Lineage P.1, Lineage B.1.429/CAL.20C, Lineage B.1.427, Lineage B.1.526, Lineage B.1.525, Lineage B.1.1.317, Lineage B.1.1.318, Lineage B.1.351, Lineage B.1.617 and Lineage P.3. In some embodiments, the SARS-CoV-2 variant described herein is a SARS-CoV-2 variant described by a Nextstrain clade selected from the group 19A, 20A, 20C, 20G, 20H, 20B, 20D, 20F, 20I, and 20E. In some embodiments, the SARS-CoV-2 virus described herein is a SARS-CoV-2 variant comprising at least one mutation selected from the group of D614G, E484K, N501Y, S477G/N, P681H, E484Q, L452R and P614R. In some embodiments, the SARS-CoV-2 variant described herein is a SARS-CoV-2 variant derived from the variants described herein. In some embodiments, the SARS-CoV-2 virus described herein is a SARS-CoV-2 variant having an at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% sequence identity to the viral genome sequence of at last one SARS-CoV-2 variant described herein.

The coronavirus infection can cause a respiratory tract infection resulting in a disease or syndrome that is a respiratory syndrome. The respiratory syndrome can be a severe acute respiratory syndrome (SARS). In some embodiments, the SARS-CoV-2 infection is at least one of the three clinical courses of infections can be distinguished: (1) mild illness with upper respiratory tract manifestations, (2) non-life-threatening pneumonia and, (3) severe condition with pneumonia, acute respiratory distress syndrome (ARDS), severe systemic inflammation, organ failures, cardiovascular complications.

The composition for use of the invention may further comprise one or more pharmaceutically acceptable diluent or carrier.

“Pharmaceutically acceptable diluent or carrier” means a carrier or diluent that is useful in preparing a pharmaceutical composition that is generally safe, non-toxic, and desirable, and includes carriers or diluents that are acceptable for human pharmaceutical use.

Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable diluent or carrier include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyl cellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

Alternatively, the pharmaceutical compositions of the invention further comprises one or more additional therapeutic agent. Preferably, the one or more therapeutic agent comprises a therapeutically effective amount of one or more nucleoside analog, protease inhibitor, immune-suppressor (e.g. sarilumab or tocilizumab), chloroquine, hydroxychloroquine antibiotic, an antibody directed against structural components of the virus, or fragment thereof (e.g. passive immunotherapy), interferon beta (e.g. interferon beta-1a) and/or a vaccine.

In certain other embodiments, the pharmaceutical compositions of the invention and the one or more additional therapeutic agent will be administered substantially simultaneously or concurrently. For example, a subject may be given a pharmaceutical composition for use of the invention while undergoing a course of treatment with the one or more additional therapeutic agent. In addition, it is contemplated that the subject has already or may be concurrently receiving other forms of antiviral therapy/ies.

In some embodiments, the additional therapeutic agent may be useful to reduce the possible side-effect(s) associated with the administration of an antibody, or an antigen-binding fragment thereof, of the invention.

In some embodiments, the additional therapeutic agent may be useful to support the effect associated with the administration of an antibody, or an antigen-binding fragment thereof, of the invention.

In some embodiments, administration of the additional therapeutic and an antibody, or an antigen-binding fragment thereof, of the invention results in a synergistic effect regarding desired effect and/or side effect.

In some embodiments, the additional therapeutic agent described herein is at least one agent selected from the group of nucleoside analog, protease inhibitor and immune modulators such as immune-suppressors.

In some embodiments, the additional therapeutic agent described herein is at least one agent selected from the group of nucleoside analog, protease inhibitor, immune-suppressor (e.g. sarilumab or tocilizumab), chloroquine, hydroxychloroquine antibiotic, an antibody directed against structural components of the virus, or fragment thereof (e.g. passive immunotherapy), interferon beta (e.g. interferon beta-1a) and/or a vaccine.

Non-limiting examples of a nucleoside analog comprise Ribavirin, Remdesivir, β-d-N4-hydroxycytidine, BCX4430, Gemcitabine hydrochloride, 6-Azauridine, Mizoribine, Acyclovir fleximer, and a combination of one or more thereof.

Non-limiting examples of protease inhibitor comprise HIV and/or HCV protease inhibitor.

In some embodiments, the immune modulator described herein is interferon beta. In some embodiments, the immune modulator described herein is interferon beta-1a. Non-limiting examples of immune-suppressor comprise interleukin inhibitors, such as for example IL-6 (e.g. sarilumab or tocilizumab), IL-1, IL-12, IL-18 and TNF-alpha inhibitors.

The additional therapeutic agents may improve or complement the therapeutic effect of compositions and methods described herein.

Accordingly, the invention is at least in part based on the finding that certain combinations (such as IFN-beta-1a with AAT) improve the effect of AAT on viral entry and inflammation.

The present invention also contemplates a gene delivery vector and pharmaceutical compositions containing the same. Preferably, the gene delivery vector is in the form of a plasmid or a vector that comprises one or more nucleic acid encoding the AAT protein, a variant, an isoform and/or a fragment thereof of the invention. Examples of gene delivery vectors comprise e.g., viral vectors, non-viral vectors, particulate carriers, and liposomes. The gene deliver is preferably performed in vitro or ex vivo.

Accordingly, the invention is at least in part based on the finding that AAT gene delivery (gene therapy) reduce the effect of inflammation.

In an embodiment, said viral vector is a vector suited for ex-vivo and in-vivo gene delivery, preferably for ex vivo gene delivery. More preferably, the viral vector is selected from the group comprising an adeno-associated virus (AAV) and a lentivirus, e.g. Lentivirus of 1st, 2nd, and 3rd generation, not excluding other viral vectors such as adenoviral vector, herpes virus vectors, etc. Other means of delivery or vehicles are known (such as yeast systems, microvesicles, gene guns/means of attaching vectors to gold nanoparticles) and are provided, in some embodiments, one or more of the viral or plasmid vectors may be delivered via liposomes, nanoparticles, exosomes, microvesicles, or a gene-gun.

In other embodiments of the invention, the pharmaceutical composition(s) of the invention is/are a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions of the present invention may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For human use, the composition may be administered as a suitably acceptable formulation in accordance with normal human practice. The skilled artisan will readily determine the dosing regimen and route of administration that is most appropriate for a particular patient. The compositions of the invention may be administered by traditional syringes, needleless injection devices, “microprojectile bombardment gone guns”, or other physical methods such as electroporation (“EP”), “hydrodynamic method”, or ultrasound. The composition can also be administered by intravenous injection, intravenous infusion, infusion with a dosator pump, inhalation nasal-spray, eye-drops, skin-patches, slow release formulations, ex vivo gene therapy or ex vivo cell-therapy, preferably by intravenous injection.

The compositions may be injected intra venously or locally injected in the lung or respiratory tract or electroporated in the tissue of interest.

The present invention further provides methods of treatment and/or prevention of a disease or syndrome related to a coronavirus infection in a subject in need thereof, the method comprising administering a therapeutically effective amount of i) an Alpha1-Antitrypsin protein, a variant, an isoform and/or a fragment thereof as described herein, or of ii) a pharmaceutical composition for use of the invention as described herein.

Further provided are methods of modulating onset of coronavirus infection in a subject exposed or suspected of being exposed to coronavirus comprising, administering to the subject in need of such a treatment a therapeutically effective amount of i) an Alpha1-Antitrypsin protein, a variant, an isoform and/or a fragment thereof as described herein, or of ii) a pharmaceutical composition for use of the invention as described herein.

The present invention also related to a for determining the susceptibility of a subject of interest for treatment and/or prevention of a disease or syndrome related to a virus infection, preferably a coronavirus infection, more preferably a SARS-CoV-2 infection using a composition comprising a therapeutically effective amount of an alpha1-antitrypsin (AAT) protein, a variant, an isoform and/or a fragment thereof as defined in herein, comprising the steps of:
a) determining the level of at least one of the group consisting of endogenous alpha1-antitrypsin, at least one spike protein priming protease, ACE2 receptor and interferon-gamma in the subject of interest prior to a virus infection, preferably a coronavirus infection, more preferably a SARS-CoV-2 infection or during a virus infection, preferably a coronavirus infection, more preferably a SARS-CoV-2 infection,
b) determining the level of at least one of the group consisting of endogenous alpha1-antitrypsin, at least one spike protein priming protease, ACE2 receptor and interferon-gamma in at least one reference subject during a virus infection, preferably a coronavirus infection, more preferably a SARS-CoV-2 infection, wherein the reference subject is asymptomatic or has mild symptoms,
c) comparing the level of interest determined in step a) to the reference level determined in step b),
   wherein the subject of interest is more susceptible for treatment and/or prevention of a disease or syndrome related to a virus infection, preferably a coronavirus infection, more preferably a SARS-CoV-2 infection if the subject of interest has at least one selected from the group consisting of:
1. a lower level of interest of endogenous alpha-antitrypsin (AAT) compared to the reference level of endogenous AAT,
2. a higher level of interest of at least one spike protein priming protease compared to the reference level of at least one spike protein priming protease,
3. a higher level of interest of angiotensin converting enzyme 2 (ACE2 receptor) compared to the reference level of the ACE2 receptor and
4. a higher level of interest of interferon-gamma (IFN-γ) compared to the reference level of IFN-γ.

All definitions and combinations provided herein apply to this embodiment, if applicable and unless indicated otherwise.

The present invention further relates to a method for determining the therapeutically effective amount of alpha1-antitrypsin (AAT) for an effective treatment and/or prevention of a disease or syndrome related to a virus infection, preferably a coronavirus infection, more preferably a SARS-CoV-2 infection using the composition for use of the present invention comprising the steps of:
a) determining the level of endogenous alpha1-antitrypsin in a subject of interest prior to a virus infection, preferably a coronavirus infection, more preferably a SARS-CoV-2 infection or during a virus infection, preferably a coronavirus infection, more preferably a SARS-CoV-2 infection,
b) determining the amount of AAT in the composition, which is required to achieve a level of AAT in the subject of at least 10 μM, preferably at least 20 μM, more preferably at least 50 μM, even more preferably at least 100 μM, and most preferably at least 200 μM.

All definitions and combinations provided herein apply to this embodiment, if applicable and unless indicated otherwise.

In some embodiments, the invention relates to the method according to the invention wherein the virus is a coronavirus. In some embodiments, the invention relates to the method according to the invention, wherein the virus is a SARS-CoV-2. In some embodiments, the invention relates to the method according to the invention, wherein the disease or syndrome is a respiratory syndrome or a severe acute respiratory syndrome. In some embodiments, the invention relates to the method according to the invention, wherein the disease or syndrome is an inflammatory disease or syndrome of the nervous system. In some embodiments, the invention relates to the method according to the invention, wherein the inflammatory disease or syndrome of the nervous system is a disease or syndrome selected from the group of multiple sclerosis, amyotrophic lateral sclerosis, Alzheimer's disease, Parkinson's disease and Huntington's disease.

All definitions and combinations provided herein apply to these embodiments, if applicable and unless indicated otherwise.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the singular form “a”, “an” and “the” include plural references unless the context clearly dictates otherwise.

As used herein, “at least one” means “one or more”, “two or more”, “three or more”, etc.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: IFNγ-mediated microglial activation

FIG. 2: AAT decreases IFNγ-mediated microglial activation

FIG. 3: Validation of AAT anti-inflammatory effect for extraction of RNA samples

FIG. 4: Validation of microglial activation and AAT anti-inflammatory effect by GSEA analysis

FIG. 5: AAT (Sigma Aldrich, batch A6150) inhibits TACE activity in a cell-free assay shown as relative fluorescence unit (A) or as percentage of the control activity (B) with an IC₅₀ of 15.3 μM (C)

FIG. 6: Results of the sciatic nerve electrophysiology (EMG) (A) Amplitude (B) conduction velocity

FIG. 7: Results of the grip strength test (A) absolute values, (B) % of 6 week timepoint

FIG. 8: Results of the rotarod test (A) absolute values, (B) % of 6 week timepoint

FIG. 9: DNAJB9 and PLA2G4B gene expression in response to AAT treatment

FIG. 10: Number of axons

FIG. 11: Axonal diameter

FIG. 12: g ratio

FIG. 13: Individual histology images of sciatic nerve semithin cross sections. Scale bar 10 μm A) Group 1: WT control, B) CMT1A+vehicle, C) CMT1A+hAAT

FIG. 14: Plasma IL-6 concentration

FIG. 15: Plasma TNFα concentration

FIG. 16: Study scheme for CMT1A mouse model and AAT administration

FIG. 17: Cell Morphology and count after treatments: SH-SY5Y morphological analysis and cell count after 6-OHDA administration and AAT treatment. Brightfield pictures (20×) and cell count (DO vs D4 of culture) showing the effect of treatments on SH-SY5Y cell phenotype and proliferation and AAT positive action. A) Example images B) Quantification

FIG. 18: Cell Viability: Graph represents cell viability measured through absorbance (450 nm) for control and samples

FIG. 19: IL-6 quantification in cell supernatant

### EXAMPLES

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all embodiments illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### Example 1

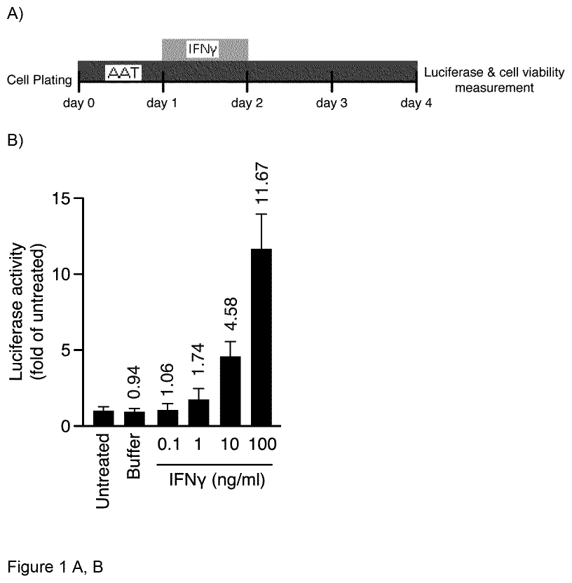

A) Human microglial cells HMC3-MHCII^{Luc} cells were plated at day 0, activated with IFNγ at day 1 until day 2 and measurement of the luciferase activity and cell viability were done at day 4.
B) Activation was measured by the activity of MHCII-driven luciferase and normalized to cell viability. Luciferase activity for all conditions is represented as fold of the untreated control. A potential effect of the highest concentration of the buffer used for drug presentation (IFNγ, AATs) was precluded. (FIG. 1). All conditions were performed in triplicates, error bars represent standard deviation.

### Example 2

A) Human microglial HMC3-MHCII^{Luc} and HMC3-MHCII^{Luc};Ubi^{AAT} cells were plated at day 0, presented IFNγ at day 1 until day 2 and measurement of the luciferase activity and cell viability were done at day 4.
B) AATs were applied from day 0 to day 4 on HMC3-MHCII^{Luc} cells. Activation was measured by the activity of MHCII-driven luciferase and normalized to cell viability. Luciferase activity for all conditions is represented as percentage of IFNγ control. All conditions were performed in triplicates, error bars represent standard deviation.

### Example 3

A) Human microglial HMC3-MHCII^{Luc} cells were plated at day 0, presented IFNγ at day 1 until day 2 and measurement of the luciferase activity and cell viability were done at day 4.
B) AATs were applied from day 0 to day 4 on HMC3-MHCII^{Luc} cells. Activation was measured by the activity of MHCII-driven luciferase and represented as percentage of IFNγ control for all conditions. All conditions were performed in triplicates, error bars represent standard deviation.
C) Bulk RNA extraction was performed on the same HMC3-MHCII^{Luc} cultures. Quality control (QC) were applied to RNA before sequencing. QC of the sequencing was done prior to mapping on the human genome. Mapped reads were counted, and differential gene expression was measured between the conditions (see Table 2-11).

### Example 4

RNAseq data from plasma-derived and recombinant AATs were pooled, normalized and analyzed through Gene Set Enrichment Analysis (GSEA) (https://www.gsea-msigdb.org/gsea/index.jsp; Subramanian, A., et al. Proc. Natl. Acad. Sci. USA, 102 (43): 15545-15550, 2005.). Upregulated gene families (grey bars) and downregulated ones (black bars) are shown according to normalized enrichment score.
A) The inflammatory profile induced by IFNγ was confirmed by upregulation of several processes related to inflammation (bold italic).
B-C) AAT treatment, in absence (B) or presence (C) of IFNγ was able to significantly downregulate some of these pathways (bold italic). Importantly, both the IFNγ and inflammatory responses were dampened by AAT. However, the downregulation of the inflammatory response genes in (C) was just under significance.

Regarding other gene families, KRAS signaling is of importance since it has been involved in oncogenic process and immunomodulation (Dias Carvalho et al. 2018). As well, p53 pathway is of note as a mediator of response to stress.

### Example 5

### AAT Treatment Counterbalances the Expression of Inflammatory Genes

Tables of genes related to antigen presentation (Table 2), cytokine signaling (Table 3), interferon signaling (Table 4) and complement activation (Table 5). Inflammatory top gene (FC>2; p-value<0.05; left column), were defined by differential expression between untreated and IFNγ-treated cells (inflammation; middle column). Top genes that were significantly and oppositely regulated by AAT treatment are highlighted (right column; bold underline).

A substantial number (˜35%) of inflammatory top genes were found affected by AAT treatment (6 of 23 genes related to antigen presentation; 14 of 33 genes related to cytokine signaling; 1 of 7 genes related to complement activation; 5/13 genes related to interferon signaling) showing its anti-inflammatory potential. For example, the promoter of HLA-DRA gene which was used as a driver for luciferase expression in the HMC3-MHCII^{Luc} line was consistently up- and down-regulated in inflammation and with AAT treatment, respectively (bold italic). Of note, FCs shown in AAT-treated inflammation condition should not be directly compared to those found in inflammation since for the former, a FC=2 represents already a 50% counterbalance on inflammation induced/repressed genes.

Second order inflammatory genes (p-value<0.05; FC<2) significantly and oppositely regulated by AAT treatment in inflammatory condition or resting condition (regulated in AAT) are presented at the bottom of the table.

TABLE 2
Antigen presentation

| | p-value | FC | p-value | FC |
|---|---|---|---|---|
| | UP in inflammation | | DOWN in inflammation + AAT | |
| Top Gene | | | | |
| CD74 | 4.15E−14 | 75.81 | | |
| LAMP3 | 9.39E−12 | 7.94 | | |
| TAP1 | 5.99E−11 | 6.12 | | |
| PSMB9 | 5.73E−11 | 5.96 | 3.11E−02 | −1.23 |
| HLA-DOB | 5.11E−04 | 4.48 | 6.82E−03 | −4.49 |
| PSMB8 | 5.18E−11 | 4.22 | | |
| HLA-DRA | 4.99E−10 | 4.76 | 4.68E−02 | −2.22 |
| SOCS1 | 1.21E−05 | 3.99 | | |
| TAP2 | 8.97E−13 | 3.54 | | |
| NCF2 | 2.57E−05 | 2.88 | | |
| CTSS | 2.29E−05 | 3.71 | | |
| HLA-DPA1 | 8.31E−09 | 3.22 | | |
| BATF3 | 1.45E−06 | 2.55 | | |
| NFKB1 | 6.21E−10 | 2.41 | | |
| IL1B | 5.54E−04 | 2.08 | 1.11E−03 | −2.07 |
| FYN | 2.18E−07 | 1.80 | | |
| BCL10 | 3.21E−05 | 1.65 | | |
| MALT1 | 1.75E−04 | 1.62 | | |
| | DOWN in inflammation | | UP in inflammation + AAT | |
| TUBA1A | 2.29E−12 | −5.00 | | |
| KIF20A | 7.29E−12 | −3.75 | | |
| CALR | 6.15E−11 | −3.53 | 4.74E−04 | 1.35 |
| PYCARD | 6.92E−03 | −2.86 | 1.53E−02 | 2.20 |
| CDC20 | 1.05E−08 | −2.80 | | |
| | DOWN in inflammation | | UP in inflammation + AAT | |
| Other Gene | | | | |
| NFATC2 | <0.05 | −1.37 | <0.05 | 1.63 |
| MRC2 | <0.05 | −1.61 | <0.05 | 1.47 |
| CTSD | <0.05 | −1.34 | <0.05 | 1.47 |
| HLA-DPB1 | <0.05 | −1.23 | <0.05 | 1.31 |
| TAPBP | <0.05 | −1.90 | <0.05 | 1.23 |
| SOCS3 | <0.05 | −1.99 | <0.05 | 1.20 |
| CTSA | <0.05 | −1.31 | <0.05 | 1.17 |
| | Regulated in inflammation | | Regulated in AAT | |
| NFATC1 | <0.05 | −1.29 | <0.05 | 1.44 |
| TRAF6 | <0.05 | 1.08 | <0.05 | −1.26 |

TABLE 3
Cytokine signalling

| | p-value | FC | p-value | FC |
|---|---|---|---|---|
| | UP in inflammation | | DOWN in inflammation + AAT | |
| Top Gene | | | | |
| IL6 | 3.53E−10 | 17.139 | | |
| TNFSF10 | 2.53E−07 | 10.595 | | |
| TRAF1 | 4.39E−07 | 7.789 | | |
| CCL5 | 5.17E−09 | 7.540 | | |
| ATF3 | 6.86E−13 | 7.218 | 1.85E−03 | −1.26 |
| PSMB9 | 6.09E−10 | 5.963 | 3.11E−02 | −1.23 |
| CEBPB | 2.57E−10 | 5.808 | | |
| CSF1 | 3.52E−09 | 5.455 | | |
| PSMB8 | 1.63E−10 | 4.219 | | |
| IL23A | 6.49E−03 | 4.218 | | |
| STAT1 | 2.04E−09 | 4.110 | | |
| IL15RA | 4.63E−07 | 3.660 | | |
| FGF2 | 1.66E−06 | 3.608 | 9.13E−03 | −1.57 |
| CXCL2 | 6.42E−05 | 3.467 | | |
| IL21R | 1.16E−05 | 3.235 | | |
| CEBPG | 3.19E−09 | 2.909 | | |
| CXCL1 | 1.36E−04 | 2.82 | 7.22E−03 | −2.09 |
| CASP1 | 1.07E−05 | 2.740 | | |
| TNFRSF9 | 2.39E−04 | 2.434 | 1.23E−03 | −2.35 |
| CXCL5 | 5.07E−05 | 2.36 | 5.98E−03 | −2.15 |
| IL1B | 2.14E−03 | 2.08 | 1.11E−03 | −2.07 |
| | DOWN in inflammation | | UP in inflammation + AAT | |
| FSCN1 | 9.42E−08 | −6.312 | 8.09E−04 | 1.48 |
| CCL2 | 2.95E−03 | −6.086 | | |
| IL4I1 | 3.22E−03 | −4.19 | 3.22E−03 | 1.95 |
| IL27RA | 2.22E−07 | −3.848 | 2.67E−02 | 1.36 |
| STAT5A | 7.66E−04 | −3.53 | 9.78E−03 | 1.45 |
| CEBPD | 5.07E−05 | −3.102 | | |
| EGF | 6.13E−03 | −2.973 | | |
| FGF18 | 2.11E−05 | −2.754 | | |
| IL6R | 6.00E−08 | −2.630 | | |
| IL1R1 | 1.27E−07 | −2.566 | 3.94E−02 | 1.18 |
| JAK3 | 3.03E−08 | −2.43 | 3.20E−03 | 1.43 |
| RASAL1 | 1.36E−06 | −2.34 | 4.65E−03 | 1.52 |
| | UP in inflammation | | DOWN in inflammation + AAT | |
| Other Gene | | | | |
| IL18 | <0.05 | 1.83 | <0.05 | −1.75 |
| NFKBIZ | <0.05 | 1.94 | <0.05 | −1.37 |
| ANXA1 | <0.05 | 1.17 | <0.05 | −1.33 |
| HBEGF | <0.05 | 1.36 | <0.05 | −1.30 |
| DUSP6 | <0.05 | 1.96 | <0.05 | −1.26 |
| | DOWN in inflammation | | UP in inflammation + AAT | |
| CX3CL1 | <0.05 | −1.38 | <0.05 | 1.61 |
| MAP2K2 | <0.05 | −1.28 | <0.05 | 1.51 |
| MAPK11 | <0.05 | −1.22 | <0.05 | 1.44 |
| TRAF2 | <0.05 | −1.66 | <0.05 | 1.42 |
| MAP2K3 | <0.05 | −1.56 | <0.05 | 1.37 |
| MAPK12 | <0.05 | −1.14 | <0.05 | 1.33 |
| MAPK13 | <0.05 | −1.27 | <0.05 | 1.23 |
| SOCS3 | <0.05 | −1.99 | <0.05 | 1.20 |
| | Regulated in inflammation | | Regulated in AAT | |
| BIRC2 | <0.05 | 1.53 | | −1.53 |
| STAT1 | <0.05 | 4.11 | | −1.47 |
| RASGRF2 | <0.05 | 1.32 | | −1.45 |
| MYD88 | <0.05 | 1.09 | | −1.35 |
| CEBPG | <0.05 | 2.91 | | −1.31 |
| RIPK2 | <0.05 | 2.03 | | −1.30 |
| TRAF6 | <0.05 | 1.14 | | −1.26 |
| SPTBN1 | <0.05 | 1.13 | | −1.21 |
| TM7SF3 | <0.05 | 1.15 | | −1.18 |
| TNFSF9 | <0.05 | −1.92 | | 1.26 |

TABLE 4
Interferon signaling

| | p-value | FC | p-value | FC |
|---|---|---|---|---|
| | UP in inflammation | | DOWN in inflammation + AAT | |
| Top Gene | | | | |
| MX2 | 7.26E−09 | 21.98 | 3.69E−03 | −2.14 |
| MX1 | 1.09E−09 | 19.39 | 3.90E−03 | −2.00 |
| IRF1 | 8.56E−12 | 8.63 | | |
| IFIT1 | 9.65E−11 | 6.73 | 3.11E−03 | −1.78 |
| ISG15 | 4.97E−06 | 5.27 | | |
| HLA-DRA | 5.04E−07 | 4.76 | 4.68E−02 | −2.22 |
| USP18 | 5.44E−08 | 4.68 | 3.59E−03 | −1.76 |
| PSMB8 | 1.63E−10 | 4.22 | | |
| STAT1 | 2.04E−09 | 4.11 | | |
| SOCS1 | 8.94E−06 | 3.99 | | |
| HLA-DPA1 | 7.92E−07 | 3.22 | | |
| IRF7 | 1.84E−04 | 2.83 | | |
| IRF5 | 2.36E−04 | 2.11 | | |
| | UP in inflammation | | DOWN in inflammation + AAT | |
| Other Gene | | | | |
| IFNAR1 | <0.05 | 1.47 | <0.05 | −1.63 |
| FLNB | <0.05 | 1.25 | <0.05 | −1.20 |
| | DOWN in inflammation | | UP in inflammation + AAT | |
| IRF3 | <0.05 | −1.45 | <0.05 | 1.56 |
| HLA-DPB1 | <0.05 | −1.23 | <0.05 | 1.31 |
| SOCS3 | <0.05 | −1.99 | <0.05 | 1.20 |
| CD44 | <0.05 | −1.14 | <0.05 | 1.14 |
| | Regulated in inflammation | | Regulated in AAT | |
| STAT1 | <0.05 | 4.11 | <0.05 | −1.47 |
| IRF2 | <0.05 | 1.24 | <0.05 | −1.17 |

TABLE 5
Complement activation

| | p-value | FC | p-value | FC |
|---|---|---|---|---|
| | UP in inflammation | | DOWN in inflammation + AAT | |
| Top Gene | | | | |
| SERPINB2 | 1.47E−05 | 25.49 | 6.63E−06 | −4.63 |
| C5AR1 | 1.78E−10 | 22.55 | | |
| C5 | 2.05E−07 | 4.22 | | |
| THBD | 1.89E−04 | 2.81 | | |
| C3 | 1.97E−06 | 2.34 | | |
| PLAU | 5.48E−09 | 2.17 | | |
| | DOWN in inflammation | | UP in inflammation + AAT | |
| PROS1 | 2.84E−08 | −3.43 | | |
| | UP in inflammation | | DOWN in inflammation + AAT | |
| Other Gene | | | | |
| C3AR1 | <0.05 | 2.44 | <0.05 | −1.50 |
| | DOWN in inflammation | | UP in inflammation + AAT | |
| PLAUR | <0.05 | −1.84 | <0.05 | 1.32 |
| ITGAX | <0.05 | −1.60 | <0.05 | 1.41 |
| | Regulated in inflammation | | Regulated in AAT | |
| PLAU | <0.05 | 2.17 | <0.05 | −1.27 |

### Example 6—AAT Treatment Enhances the Expression of Hallmark Genes Related to M2 Anti-Inflammatory Microglia

M2 microglia gene expression is promoted following a M2-type induction (FC study; Satoh 2017). AAT treatment in resting microglia (FC AAT) and in activated microglia (FC inflammation+AAT) was able to similarly enhance expression of M2 genes, while at lower magnitude. ˜60% of the modified genes were common among AAT treatment conditions (bold underline).

TABLE 6

| | | | | | FC |
|---|---|---|---|---|---|
| | | | | | inflammation + |
| M2 Gene | FC study | FC AAT | M2 Gene | FC study | AAT |
| MMP1 | 7.36 | 2.51 | HLA-DRB5 | 53.99 | 2.09 |
| ITGA11 | 4.78 | 1.65 | ANPEP | 8.90 | 1.25 |
| COL1A1 | 4.38 | 1.58 | THBS1 | 8.16 | 1.34 |
| TAGLN | 2.79 | 1.37 | MMP1 | 7.36 | 2.39 |
| GUK1 | 2.76 | 1.57 | BGN | 6.68 | 1.27 |
| PAPPA | 2.74 | 1.24 | ITGA11 | 4.78 | 1.54 |
| PLTP | 2.52 | 1.28 | OLFML2B | 4.78 | 1.44 |
| NTN4 | 2.38 | 1.25 | GUK1 | 2.76 | 1.31 |
| FOXC1 | 2.37 | 1.26 | FAT1 | 2.75 | 1.34 |
| PTPRS | 2.34 | 1.27 | PAPPA | 2.74 | 1.31 |
| COL11A1 | 2.30 | 1.14 | KDM2B | 2.69 | 1.17 |
| STC2 | 2.29 | 1.42 | CLMP | 2.46 | 1.22 |
| MXRA8 | 2.28 | 1.40 | PTPRS | 2.34 | 1.23 |
| TEKT4P2 | 2.17 | 2.46 | COL11A1 | 2.30 | 1.16 |
| SART1 | 2.11 | 1.44 | STC2 | 2.29 | 1.32 |
| FZD2 | 2.09 | 1.39 | MXRA8 | 2.28 | 1.49 |
| AP2A2 | 2.04 | 1.26 | TGFBI | 2.19 | 1.18 |
| FEZ1 | 1.99 | 1.45 | LOXL2 | 2.10 | 1.25 |
| ACAN | 1.98 | 1.37 | FZD2 | 2.09 | 1.37 |
| TGFB1I1 | 1.94 | 1.31 | FEZ1 | 1.99 | 1.34 |
| NID1 | 1.90 | 1.16 | AHNAK2 | 1.96 | 1.40 |
| RASA4B | 1.87 | 1.46 | TGFB1I1 | 1.94 | 1.26 |
| GREM1 | 1.82 | 1.69 | CLIP3 | 1.89 | 1.25 |
| FGD3 | 1.82 | 1.76 | SH3PXD2B | 1.88 | 1.19 |
| GADD45GIP1 | 1.70 | 1.55 | IL1R1 | 1.84 | 1.18 |
| MOSPD3 | 1.69 | 1.51 | GREM1 | 1.82 | 1.56 |
| APRT | 1.69 | 1.16 | FGD3 | 1.82 | 2.31 |
| RAB3IL1 | 1.68 | 1.55 | ARHGDIB | 1.79 | 1.28 |
| TNFRSF1B | 1.67 | 1.46 | GATA6 | 1.78 | 1.47 |
| SLC22A5 | 1.66 | 1.22 | PRSS23 | 1.78 | 1.16 |
| ENG | 1.66 | 1.29 | KANK2 | 1.74 | 1.25 |
| SEMA3B | 1.65 | 1.50 | GADD45GIP1 | 1.70 | 1.51 |
| DBN1 | 1.65 | 1.42 | LDHA | 1.67 | 1.21 |
| IGFBP2 | 1.63 | 1.63 | TNFRSF1B | 1.67 | 1.23 |
| TBCD | 1.63 | 1.26 | ENG | 1.66 | 1.35 |
| METRN | 1.63 | 2.23 | SEMA3B | 1.65 | 1.39 |
| SERPINH1 | 1.62 | 1.21 | DBN1 | 1.65 | 1.31 |
| MBOAT7 | 1.61 | 1.44 | IGFBP2 | 1.63 | 1.34 |
| PREX1 | 1.61 | 1.25 | KIAA1549L | 1.63 | 1.44 |
| TTC9 | 1.61 | 1.65 | TBCD | 1.63 | 1.26 |
| RASAL1 | 1.60 | 1.52 | METRN | 1.63 | 1.64 |
| ZIC2 | 1.60 | 2.42 | PLLP | 1.62 | 1.46 |
| CHPF | 1.58 | 1.80 | SERPINH1 | 1.62 | 1.20 |
| PLEC | 1.58 | 1.69 | MBOAT7 | 1.61 | 1.37 |
| APLP1 | 1.58 | 1.41 | PREX1 | 1.61 | 1.28 |
| HSPG2 | 1.57 | 1.58 | RASAL1 | 1.60 | 1.45 |
| BAIAP2 | 1.55 | 1.45 | ZIC2 | 1.60 | 2.77 |
| MIF | 1.55 | 1.83 | ZNF319 | 1.59 | 1.25 |
| FZD7 | 1.55 | 1.24 | CHPF | 1.58 | 1.58 |
| MARCH4 | 1.54 | 1.28 | PLEC | 1.58 | 1.54 |
| PRSS36 | 1.52 | 1.58 | HSPG2 | 1.57 | 1.41 |
| C16orf45 | 1.51 | 1.31 | TCEAL2 | 1.56 | 1.30 |
| EXTL1 | 1.50 | 2.19 | BAIAP2 | 1.55 | 1.24 |
| | | | MIF | 1.55 | 1.72 |
| | | | FZD7 | 1.55 | 1.36 |
| | | | FADS2 | 1.54 | 1.20 |
| | | | HCK | 1.51 | 1.44 |
| | | | EXTL1 | 1.50 | 1.78 |
| | | | SLC16A3 | 1.50 | 1.65 |

### Example 7—AAT Treatment Affect the Expression of Neurodegenerative Diseases Risk Genes

Risk genes related to PD (21 genes), AD (15 genes), MS (53 genes), MCT (50 genes) PN (88 genes) and GBS (30 genes; FC) were extracted from public libraries (Timmerman, Strickland, and Zuchner 2014; Parnell and Booth 2017; Nikolac Perkovic and Pivac 2019; Blauwendraat, Nails, and Singleton 2020; Chang et al. 2012) and their expression in AAT-treated resting microglia (FC AAT) and activated microglia (FC inflammation+AAT) was assessed. Common modified genes among AAT treatment conditions are highlighted (bold underline). Of note, the expression of several risk genes in all abovementioned diseases were modified by AAT.

TABLE 7

| Risk gene | AAT | | Risk gene | Inflammation + AAT | |
|---|---|---|---|---|---|
| PD | p-value | FC | PD | p-value | FC |
| PLA2G6 | 9.48E−03 | 1.55 | ATP13A2 | 6.94E−03 | 1.49 |
| ATP13A2 | 1.92E−02 | 1.42 | PLA2G6 | 4.87E−02 | 1.37 |
| LRP10 | 4.49E−02 | 1.29 | LRP10 | 3.15E−02 | 1.23 |
| DNAJC6 | 1.23E−02 | −1.14 | PINK1 | 4.12E−02 | 1.20 |
| GIGYF2 | 1.29E−02 | −1.16 | GBA | 4.81E−02 | 1.15 |
| SYNJ1 | 1.08E−02 | −1.33 | UCHL1 | 3.66E−02 | −1.10 |
| DNAJC13 | 3.07E−03 | −1.37 | DNAJC6 | 3.43E−02 | −1.12 |
| VPS13C | 1.84E−02 | −1.41 | SYNJ1 | 4.94E−02 | −1.23 |
| TMEM230 | 3.43E−03 | −1.42 | | | |
| VPS35 | 2.46E−03 | −1.42 | | | |
| LRRK2 | 1.98E−02 | −3.98 | | | |

TABLE 8

| Risk gene | AAT | | Risk gene | Inflammation + AAT | |
|---|---|---|---|---|---|
| AD | p-value | FC | AD | p-value | FC |
| TREM2 | 2.50E−03 | 2.22 | APOE | 3.13E−02 | 1.55 |
| ABCA7 | 3.77E−04 | 1.75 | TREM2 | 4.38E−02 | 1.47 |
| APOE | 1.05E−02 | 1.74 | BIN1 | 4.26E−03 | 1.43 |
| PSEN2 | 3.51E−03 | 1.45 | ABCA7 | 2.26E−02 | 1.36 |
| BIN1 | 3.90E−02 | 1.33 | PSEN2 | 1.00E−02 | 1.28 |
| CLU | 5.24E−03 | −1.30 | PICALM | 3.38E−02 | −1.29 |
| CD2AP | 1.25E−02 | −1.39 | | | |
| PICALM | 4.42E−03 | −1.48 | | | |

TABLE 9

| Risk gene | AAT | | Risk gene | Inflammation + AAT | |
|---|---|---|---|---|---|
| MS | p-value | FC | MS | p-value | FC |
| CYP24A1 | 8.25E−06 | 2.55 | DKKL1 | 4.05E−02 | 2.12 |
| HLA-A | 1.88E−03 | 1.48 | HLA-A | 2.42E−05 | 1.57 |
| MPV17L2 | 3.93E−02 | 1.31 | HLA-DRB1 | 1.41E−02 | 1.51 |
| AGAP2 | 3.00E−02 | 1.30 | ZMIZ1 | 8.06E−04 | 1.47 |
| CDC37 | 4.54E−02 | 1.26 | AGAP2 | 1.92E−02 | 1.45 |
| METTL1 | 3.26E−02 | 1.22 | CYP24A1 | 1.66E−02 | 1.34 |
| CD40 | 2.67E−02 | 1.17 | MPHOSPH9 | 2.92E−02 | −1.19 |
| RPS6KB1 | 4.07E−02 | −1.23 | NFKBIZ | 4.75E−02 | −1.37 |
| CBLB | 8.31E−03 | −1.24 | SLC30A7 | 3.20E−02 | −1.56 |
| PTGER4 | 3.38E−02 | −1.29 | | | |
| PTPRK | 1.93E−04 | −1.38 | | | |
| AHI1 | 6.58E−03 | −1.46 | | | |
| HLA-DRB1 | 3.40E−02 | −1.47 | | | |
| CD58 | 4.85E−02 | −1.61 | | | |
| EXTL2 | 2.52E−03 | −1.76 | | | |
| SLC30A7 | 9.06E−03 | −1.77 | | | |

TABLE 10

| Risk gene | AAT | | Risk gene | Inflammation + AAT | |
|---|---|---|---|---|---|
| MCT*/PN | p-value | FC | MCT*/PN | p-value | FC |
| SCN11A | 2.65E−02 | 4.49 | TRPV4* | 2.02E−02 | 1.72 |
| KIF1A | 1.99E−02 | 2.76 | HSPB1* | 1.06E−02 | 1.56 |
| TRPV4* | 1.14E−03 | 1.92 | TUBB3 | 2.33E−02 | 1.44 |
| HSPB1* | 2.22E−02 | 1.67 | TRPV4* | 4.70E−02 | 1.40 |
| PLEKHG5* | 4.11E−03 | 1.49 | LMNA* | 9.07E−03 | 1.39 |
| IGHMBP2* | 9.58E−03 | 1.39 | MED25* | 2.22E−02 | 1.38 |
| DNAJB2* | 9.15E−03 | 1.39 | INF2 | 4.80E−02 | 1.35 |
| LRSAM1* | 9.19E−03 | 1.36 | NDRG1* | 3.75E−03 | 1.30 |
| IFRD1 | 2.59E−02 | 1.23 | DST | 4.71E−02 | 1.30 |
| ARHGEF10 | 3.57E−03 | 1.19 | SEPT9 | 1.36E−02 | 1.29 |
| WNK1 | 4.34E−02 | −1.14 | IGHMBP2* | 3.80E−02 | 1.21 |
| CCT5 | 1.18E−02 | −1.17 | DNAJB2* | 2.76E−02 | 1.20 |
| SBF2 | 3.60E−03 | −1.20 | CCT5 | 1.91E−02 | −1.13 |
| KIF1B* | 5.00E−03 | −1.21 | KARS* | 2.35E−02 | −1.13 |
| GARS* | 1.91E−03 | −1.21 | TFG* | 1.91E−02 | −1.19 |
| GAN* | 2.14E−02 | −1.21 | GARS* | 1.12E−03 | −1.24 |
| SETX | 2.68E−02 | −1.23 | GDAP1* | 1.32E−02 | −1.24 |
| SPTLC1 | 3.73E−02 | −1.23 | SCN9A | 1.99E−03 | −1.66 |
| TFG* | 2.73E−03 | −1.28 | | | |
| DST | 2.99E−02 | −1.35 | | | |
| MTMR2* | 3.72E−03 | −1.36 | | | |
| TRIM2* | 2.09E−03 | −1.39 | | | |
| DYNC1H1* | 1.10E−02 | −1.40 | | | |
| FIG4* | 1.84E−03 | −1.42 | | | |
| SCN9A | 1.08E−02 | −1.50 | | | |
| GDAP1* | 5.03E−05 | −1.54 | | | |
| GNB4* | 6.11E−03 | −1.56 | | | |

TABLE 11

| | | AAT | | | | AAT | |
|---|---|---|---|---|---|---|---|
| Risk gene | FC | | FC | Risk gene | FC | | FC |
| GBS | GBS | p-value | AAT | GBS | GBS | p-value | AAT |
| GUK1 | −2.13 | 1.38E−02 | 1.57 | GUK1 | −2.13 | 3.31E−02 | 1.31 |
| HAGH | −2.14 | 1.07E−02 | 1.40 | NFIL3 | 2.83 | 6.38E−03 | −1.22 |
| GLRX5 | −2.48 | 4.32E−02 | 1.12 | ZNF12 | 2.79 | 4.52E−02 | −1.23 |
| HMGB2 | 3.63 | 7.91E−03 | −1.20 | FOS | 4.02 | 1.19E−02 | −1.54 |
| ZNF12 | 2.79 | 3.05E−02 | −1.26 | MARCKS | 2.81 | 2.06E−03 | −1.84 |
| CDC42 | 2.88 | 8.15E−03 | −1.35 | LY96 | 3.27 | 1.72E−02 | −2.31 |
| SMCHD1 | 2.81 | 3.33E−02 | −1.39 | | | | |
| PKN2 | 2.77 | 1.66E−02 | −1.47 | | | | |
| SENP6 | 2.76 | 1.91E−02 | −1.47 | — | — | — | — |
| MARCKS | 2.81 | 5.40E−04 | −2.06 | | | | |
| FOS | 4.02 | 6.60E−04 | −2.31 | | | | |

### Example 8

Further experiments include CSF-1 treatment for (Mϕ) to M1 macrophage transition optimization, Dose dependence of IFNγ for M1 macrophage activation, AAT treatment on resting and IFNγ-activated macrophage and RNA extraction and RNAseq and analysis. Whereby the cells are human primary resting (Mϕ) or M1-differentiated macrophages. The treatment includes pre-treatment for 24 h with AATs, followed by cell activation (or not) with CSF-1 or IFN in presence of AAT for 24 h. Cells are further treated 48 h in AAT and finally tested for pro-inflammatory cytokine release (IL-6, TNFα, IL-13, IL-8; multiplexing readout) using the culture supernatant or alternatively test for Luciferase activity (MHCII^{Luc} line) and concomitantly extract RNA for microarray analysis. Cultures with consistent readouts for the cytokine release or the luciferase assay will be used as samples for RNA extraction and microarray analysis.

Experimental conditions: every condition is performed in triplicate,
Untreated (no AAT, no CSF-1) Resting macrophage control
IFNγ: activated macrophage control
AAT (Plasma-derived; Sigma or recombinant; Lonza): AAT effect on resting macrophage
IFNγ and AAT (Plasma-derived; Sigma or Lonza): AAT anti-inflammatory effect on activated macrophage

### Output:

Resting state macrophage gene expression
Pro-inflammatory differentially-regulated genes (fold of untreated; significant p-value; fold up/down-regulation threshold)
AAT-driven gene expression change on resting and activated macrophage
Difference between recombinant and plasma-derived AAT gene regulation

### Methods

### Human Microglial Cell Line Culture

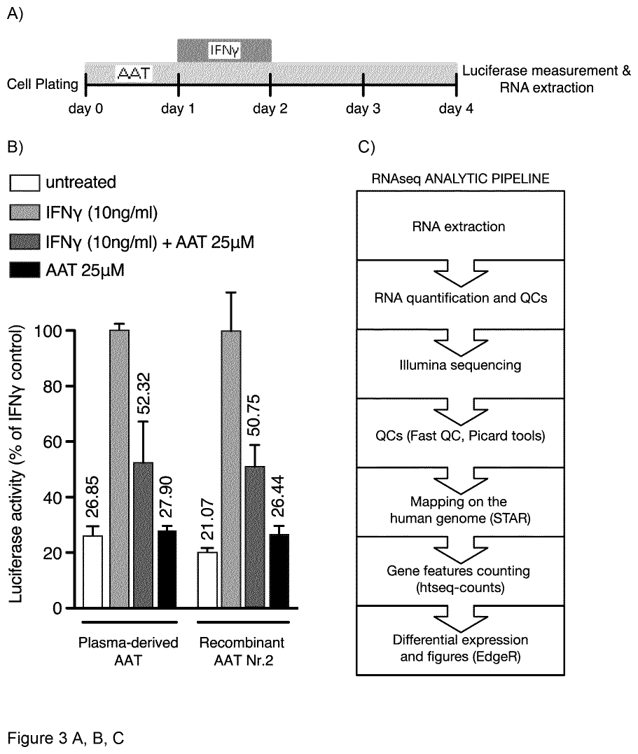

HMC3-MHCII^{Luc} cell line coding for Renilla luciferase under major histocompatibility complex II promoter (HLA-DRA) has been described as a valuable tool to study human microglial activation by and was obtained from Prof. Karl-Heinz Krause, University of Geneva. It was transduced with a lentiviral vector to obtain the HMC3-MHCII^{Luc}; Ubi^{AAT} cell line (See FIG. 3). Both HMC3-MHCII^{Luc} and HMC3-MHCII^{Luc}; Ubi^{AAT} cell lines were cultured on TC treated cell culture dishes (CELLSTAR®, Greiner, 7.664160) in DMEM high glucose+glutamine (Gibco, 41965039) supplemented with 10% (v/v) fetal bovine serum (FBS, Gibco, 10270106) and 100 pg/ml penicillin/streptomycin (Pen/Strep, ThermoFisher, 15070063). Cultures were maintained at 37° C. in a 5% C02 atmosphere. Passage was done by quickly rinsing the cells in PBS 1×, 3 min trypsinization at RT (Tryple Express, ThermoFisher, 12604021) followed by centrifugation (5 min, 1000 RPM) and resuspension in the abovementioned supplemented DMEM. Cells were counted and plated at desired concentration.

### Human Microglial Cell Line Transduction

The lentivirus coding for human AAT under ubiquitin promoter and GFP under human PGK promoter was obtained according to the protocol described in Marc Giry-Laterrière, Els Verhoeyen, and Patrick Salmon, 2011, Methods in molecular biology. In brief, 4.5×10⁶ HEK cells were plated in a 0100 mm dish and transfected 16 h later with 15 pg of pCWXPG-UBI-SP::AAT, 10 pg of packaging plasmid (psPAX2, gift from Didier Trono [Addgene plasmid 12260]), and 5 pg of envelope (pMD2G, gift from Didier Trono [Addgene plasmid 12259]). The medium was changed 8 h post-transfection. After 48 h, viral supernatant was collected and filtered using 45 μm PVDF filters and stored at −80° C. Titer of the virus was done and HMC3-MHCII^{Luc}; Ubi^{AAT} cell lines with approximately 100% and 50% of cells expressing the AAT were selected for experimental conditions.

### IFNγ-Mediated Human Microglial Activation

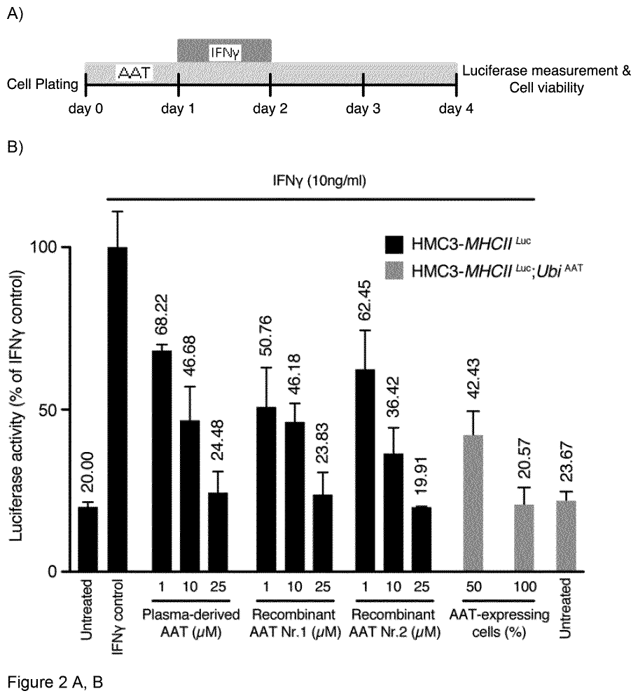

HMC3-MHCII^{Luc} cell line was seeded into 96-well plates at a density of approximately 2500 cells/well. 24 h after, their activation was induced with a 24 h-long IFNγ (Sigma, SRP3058) presentation at ranged concentrations (0.1; 1; 10 or 100 ng/ml). IFNγ was then removed and cells cultured for 48 h before being assessed for cell viability and activation (see FIG. 2).

### Exogenous/Endogenous AATs Treatment on IFNγ-Activated Human Microglia

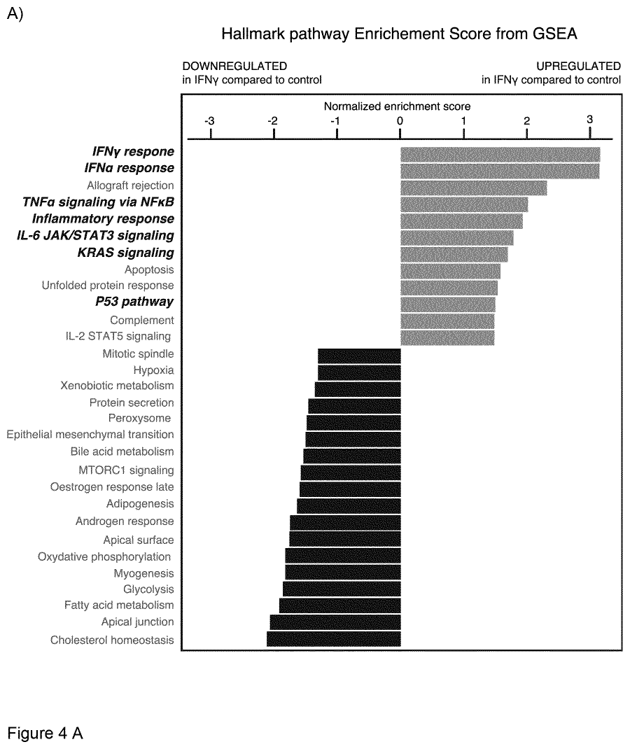

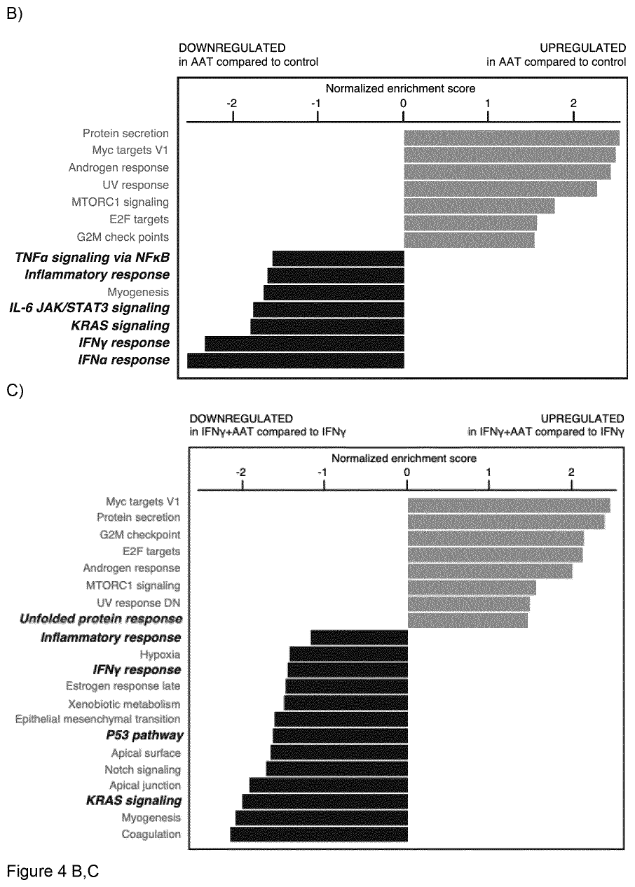

HMC3-MHCII^{Luc} and HMC3-MHCII^{Luc}; Ubi^{AAT} (endogenous AAT) cell lines were seeded into 96-well plates at a density of approximately 2500 cells/well. HMC3-MHCII^{Luc} cell line was plated and was added plasma-derived AAT and recombinant AATs (produced in CHO cells, AAT 1 and AAT 2) 3 h later and at ranged concentration (1; 10 or 25 μM). 24 h after, still in presence of exogenous or endogenous AAT, microglial activation was induced with a 24 h-long IFNγ presentation (10 ng/ml). IFNγ was then removed and both HMC3-MHCII^{Luc} and HMC3-MHCII^{Luc}; Ubi^{AAT} cells were cultured for 48 h in exogenous or endogenous presence of AAT before cell cultures were being assessed for cell viability and activation (See FIGS. 3 and 4).

### Human Microglia Cell Viability and Activation Measurement

Viability (Cell Counting Kit-8, Sigma, 96992) and activation (Renilla-Glo® Luciferase Assay System, Promega, E2710) of HMC3-MHCII^{Luc} and HMC3-MHCII^{Luc}; Ubi^{AAT} cell cultures were measured according to the manufacturers' protocols.

### RNA Collection, Sequencing and Differential Expression Analysis

RNA extraction was achieved with RNeasy Mini kit (Qiagen) according to manufacturer's protocol. RNA samples from plasma-derived AAT and recombinant AAT Nr. 2 were checked for quality (2100 Bioanalyzer, Agilent) and libraries prepared with Truseq RNA Library Kit (Illumina, RS-122-2001). Libraries were sequenced (HiSeq 4000, Illumina) controlled for the quality of sequencing (FastQC), mapped on the human genome (STAR v.2.7.0f; UCSC hg38), reads were counted (HTSeq v0.9.1) and the differential expression analysis was performed with the R/Bioconductor package (edgeR 1.30.1.).

### RNA Collection, Sequencing and Differential Expression Analysis

Human monocyte-derived M1 macrophage (GM-CSF, PromoCell, C-12916) were cultured on fibronectin-coated cell culture dishes in M1-Macrophage Generation Medium XF and activated with CSF-1 (50 ng/ml, Sigma, SRP3058) according to the manufacturer's protocol, Cultures were maintained at 37° C. in a 5% C02 atmosphere.

### Cytokine Multiplex Assay

Cell culture supernatant was collected and measure for IL-6, TNFα, IL-1β and IL-8 with bead based Luminex assay according to the manufacturer's protocol.

### Cell Free TACE/ADAM17 Activity

TACE activity and its inhibition by human AAT (AAT) was performed with Recombinant Human TACE/ADAM17 kit (930-ADB and ES003, R&D Systems) in black 96-well immuno plates (437111, ThermoFisher Scientific). The enzymatic activity of TACE/ADAM17 was measured by mixing 0.005 μg of rhTACE with 10 μM of Mca-PLAQAV-Dpa-RSSSR-NH2 fluorogenic peptide substrate III in assay buffer (25 mM Tris, 2.5 μM ZnCl2, 0.005% Brij-35 (w/v), pH 9.0) to a final volume of 100 μl. AAT (Sigma Aldrich, batch A6150) was resuspended in water (vehicle), control TACE/ADAM17 activity was assessed in presence of the vehicle (amount used for AAT 100 μM). AAT was added at different concentration (0, 6.25, 12.5, 25, 50 and 100 μM) to assess its dose-dependent inhibition of TACE/ADAM17. All conditions were performed in triplicates. Activity was measured as relative fluorescent unit (RFU) in a kinetic mode (9 time points over 5 min) with a SpectraMax iD3 Microplate Reader (low PMT gain, is exposition, top read at 1 mm, wavelength: excitation 320 nm, emission 405 nm). Bar graph as percentage of control activity was obtained by averaging the values obtained over the 5 min for each of the condition.

### Animals

As a murine model of CMT1A we used C3-PMP22 transgenic mice (B6.Cg-Tg(PMP22)C3Fbas/J, The Jackson Laboratory) which express three copies of a wild-type human peripheral myelin protein 22 (PMP22) gene (Verhamme, King et al. 2011). Mice were housed in macrolon cages with filter hoods, in a continuously air-filtered room, thereby avoiding contamination. During experiments, paired animals will be caged at a constant temperature with a day/night cycle of 12/12 hours. Animals were fed (control tap water and nutrition) ad libitum. Animal protocol is approved by the Animal Studies Committee of Languedoc Roussillon. This protocol and our laboratory procedures comply with French legislation, which implements the European Directives (Reference Number: D3417223, APAFIS #23920-2020020320279696 v3). Animal health is followed on a daily basis to ensure that only animals in good health enter the testing procedures and follow up the study.

### In Vivo Study Paradigm

Animals were split in 3 groups (wild-type control (subcutaneous 0.9% NaCl), CMT1A-vehicle (subcutaneous 0.9% NaCl), CMT1A-human alpha-1 antitrypsin (subcutaneous, twice daily, 50 mg/kg per injection)) of 3 mice each (3 weeks old males of 18±2.5 g at the beginning of study) and all went through the following protocol after 7 days of acclimation on site.

Starting from the age of 4 weeks, animals were subjected to blood samplings for the determination of interleukin-6 (IL-6) and tumor necrosing factor alpha (TNFα) levels, as described in FIG. 16.

Plasma levels of hAAT were evaluated every 5 days from the first day of treatment to the last treatment day.

On the first and last day of treatment, the neuromuscular performance of animals was tested with a rotarod test, grip test and sciatic nerve electrophysiology test. After the last treatment at 8 weeks, these tests were repeated, and animals were sacrificed and the left sciatic nerve was sampled for histological evaluation of the number and size of neurons.

### Example 9

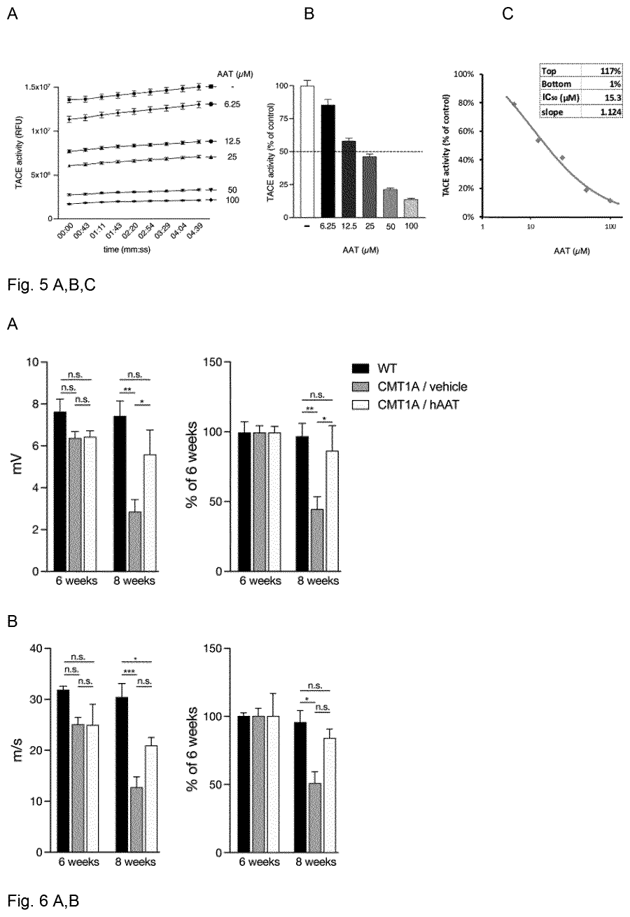

TACE activity is assessed according to the manufacturer's instruction (Recombinant Human TACE/ADAM17 kit, 930-ADB, R&D Systems) in kinetic mode, without or with different AAT concentration. All conditions were done in triplicates and are shown as mean±SD (FIG. 5).

### Example 10

The most common type of CMT is CMT1A, characterized by a duplication of the PMP22 gene leading to an accumulation of the pmp22 protein in the Schwann cell and progressive demyelination. PMP22 is a tetraspan glycoprotein contained in compact myelin of the peripheral nervous system. Duplication of PMP22 has been associated with the onset of Charcot-Marie-Tooth disease type 1A (CMT1A). The C3-PMP22 transgenic mice (B6.Cg-Tg(PMP22)C3Fbas/J) express three copies of a wildtype human peripheral myelin protein 22 (PMP22) gene. The cause and effect between the additional PMP22 gene and CMT1A are still not well understood and remain elusive to this day. Several plausible hypotheses are, nevertheless, available to link the genetic abnormality, that is the duplication of the PMP22 gene, to the pathology's manifestation. Without being bound to theory, PMP22 overexpression may exert a negative effect on the formation of myelin sheaths in the peripheral nervous system (PNS). These mice present an age-dependent demyelinating neuropathy characterized by predominantly distal loss of strength and sensation. C3-PMP mice show no overt clinical signs at 3 weeks and develop progressive and observable neuromuscular impairment after 4 weeks. The mice have stable, low nerve conduction velocities the same way as in adults with human CMT1A. Myelination is delayed in these mice, and they contain reduced numbers of myelinated fibers at 3 weeks of age. This mouse model was used to study the effect of AAT in different paradigms.

Positive efficacy of AAT administration was observed after two weeks in the CMT1A mice by increasing rotarod latency, grip strength and nerve conduction performances compared to an untreated control group. Moreover, there is no observable body weight loss in the AAT treated group compared to the vehicle group suggesting the absence of systemic toxicology of the compound at these experimental conditions.

TABLE 12
Body weight
WT control

| | 4 weeks old | 5 weeks old | 6 weeks old | 7 weeks old | 8 weeks old |
|---|---|---|---|---|---|
| Mouse 1.1 | 18.6 | 19.5 | 19.8 | 20.2 | 21.1 |
| Mouse 1.2 | 19.1 | 19.7 | 19.8 | 20.3 | 20.9 |
| Mouse 1.3 | 18.4 | 18.9 | 19.4 | 19.8 | 20.6 |
| MEAN | 18.70 | 19.37 | 19.66 | 20.12 | 20.90 |
| SD | 0.36 | 0.40 | 0.20 | 0.25 | 0.25 |
| SEM | 0.21 | 0.23 | 0.12 | 0.14 | 0.14 |
| | 6 weeks old | 8 weeks old | 6 weeks old | 8 weeks old | 6 weeks old |

CMT1A + vehicle

| | | | | | |
|---|---|---|---|---|---|
| Mouse 2.1 | 19.5 | 20.4 | 20.8 | 21.3 | 21.4 |
| Mouse 2.2 | 19.2 | 19.8 | 20.4 | 20.6 | 21.2 |
| Mouse 2.3 | 18.1 | 18.4 | 19.0 | 19.7 | 20.5 |
| MEAN | 18.93 | 19.57 | 20.08 | 20.51 | 21.05 |
| SD | 0.74 | 1.03 | 0.98 | 0.79 | 0.49 |
| SEM | 0.43 | 0.60 | 0.57 | 0.46 | 0.29 |

CMT1A + hAAT

| | | | | | |
|---|---|---|---|---|---|
| Mouse 3.1 | 18.7 | 19.2 | 19.6 | 20.1 | 20.8 |
| Mouse 3.2 | 19.2 | 20.1 | 20.5 | 21.1 | 21.8 |
| Mouse 3.3 | 18.6 | 18.6 | 19.1 | 19.9 | 20.6 |
| MEAN | 18.83 | 19.31 | 19.73 | 20.33 | 21.06 |
| SD | 0.32 | 0.78 | 0.74 | 0.63 | 0.62 |
| SEM | 0.19 | 0.45 | 0.43 | 0.36 | 0.36 |

Sciatic nerve electrophysiology (EMG) provides sensitive and quantitative approach to measure compound muscle action potential and nerve conduction velocity amplitude in the animals and was done by stimulation of the sciatic nerve. Similar compound muscle action potential (CMAP) amplitudes were observed between groups at the baseline (6 weeks old). As expected, a strong and significant decrease of CMAP amplitude was observed in the CMT1A+vehicle group compared to the wild type control group at 8 weeks old. Results show improvement of the EMG parameters for CMT1A mice treated with AAT compared to the control. (FIG. 6) suggesting a positive efficacy of hAAT on axonal degeneration induced by CMT1A disorder.

Lower nerve conduction velocities (NCV) were observed in both CMT1A groups compared to the wild type control group at the baseline (6 weeks old). At the baseline, the differences of NCVs between groups were not statistically significant. As expected, a strong and significant decrease of NCV was observed in the CMT1A+vehicle group compared to the wild type control group at 8 weeks old. The CMT1A+hAAT treated group presented an increase of the NCV compared to the vehicle treated group. Because the nerve conduction velocity depends on the myelin sheath integrity, these data also suggest a positive efficacy of hAAT on the Schwann cell demyelination induced by CMT disorder.

TABLE 13
Sciatic nerve electrophysiology
WT control

| | Amplitude (mV) | | T1 distal (s) | | T2 proximal (s) | | Conduction velocity (m/s) | |
|---|---|---|---|---|---|---|---|---|
| | 6 weeks old | 8 weeks old | 6 weeks old | 8 weeks old | 6 weeks old | 8 weeks old | 6 weeks old | 8 weeks old |
| Mouse 1.1 | 8.881 | 7.462 | 0.00251 | 0.00456 | 0.00298 | 0.00508 | 31.501 | 28.846 |
| Mouse 1.2 | 6.982 | 6.204 | 0.00331 | 0.00426 | 0.00376 | 0.00468 | 33.523 | 35.937 |
| Mouse 1.3 | 7.140 | 8.721 | 0.00271 | 0.00739 | 0.00319 | 0.00795 | 31.071 | 26.986 |
| MEAN | 7.668 | 7.462 | | | | | 32.032 | 30.590 |
| SD | 1.054 | 1.259 | | | | | 1.309 | 4.723 |
| SEM | 0.608 | 0.727 | | | | | 0.756 | 2.727 |

CMT1A + vehicle

| | Amplitude (mV) | | T1 distal (s) | | T2 proximal (s) | | Conduction velocity (m/s) | |
|---|---|---|---|---|---|---|---|---|
| | 6 weeks old | 8 weeks old | 6 weeks old | 8 weeks old | 6 weeks old | 8 weeks old | 6 weeks old | 8 weeks old |
| Mouse 2.1 | 6.942 | 1.785 | 0.00228 | 0.00215 | 0.00292 | 0.00354 | 23.644 | 10.720 |
| Mouse 2.2 | 6.452 | 3.162 | 0.00293 | 0.00235 | 0.00357 | 0.00379 | 23.593 | 10.392 |
| Mouse 2.3 | 5.835 | 3.735 | 0.00137 | 0.00213 | 0.00191 | 0.00302 | 27.863 | 16.923 |
| MEAN | 6.410 | 2.894 | | | | | 25.034 | 12.678 |
| SD | 0.555 | 1.002 | | | | | 2.451 | 3.680 |
| SEM | 0.320 | 0.579 | | | | | 1.415 | 2.124 |

CMT1A + hAAT

| | Amplitude (mV) | | T1 distal (s) | | T2 proximal (s) | | Conduction velocity (m/s) | |
|---|---|---|---|---|---|---|---|---|
| | 6 weeks old | 8 weeks old | 6 weeks old | 8 weeks old | 6 weeks old | 8 weeks old | 6 weeks old | 8 weeks old |
| Mouse 3.1 | 7.0295 | 3.7994 | 0.00202 | 0.00251 | 0.00250 | 0.00313 | 31.277 | 23.904 |
| Mouse 3.2 | 6.0254 | 5.2600 | 0.00207 | 0.00236 | 0.00295 | 0.00318 | 17.182 | 18.310 |
| Mouse 3.3 | 6.3586 | 7.8216 | 0.00160 | 0.00228 | 0.00217 | 0.00302 | 26.268 | 20.464 |
| MEAN | 6.4712 | 5.6270 | | | | | 24.909 | 20.892 |
| SD | 0.5114 | 2.0360 | | | | | 7.145 | 2.821 |
| SEM | 0.295 | 1.176 | | | | | 4.125 | 1.629 |

TABLE 14
Mean compound muscle action potential
Compound muscle action potential, mV (Mean ± SEM)

| | 6 weeks old | 8 weeks old |
|---|---|---|
| WT control | 7.67 ± 0.61 | 7.46 ± 0.73 |
| CMT1A + vehicle | 6.41 ± 0.32 | 2.89 ± 0.58*** |
| CMT1A + AAT | 6.47 ± 0.30 | 5.63 ± 1.18^{†} |

2-way ANOVA with repeated measures and Bonferroni t-test
***p < 0.001 vs WT control;
^{†}p < 0.05 vs CMT1A + vehicle

TABLE 15
Mean nerve conduction velocity
Nerve conduction velocity, m/s (Mean ± SEM)

| | 6 weeks old | 8 weeks old |
|---|---|---|
| WT control | 32.03 ± 0.76 | 30.59 ± 2.73 |
| CMT1A + vehicle | 25.03 ± 1.42 | 12.68 ± 2.12*** |
| CMT1A + AAT | 24.91 ± 4.13 | 20.89 ± 1.63* |

2-way ANOVA with repeated measures and Bonferroni t-test
**; ***p < 0.01; p < 0.001 vs WT control

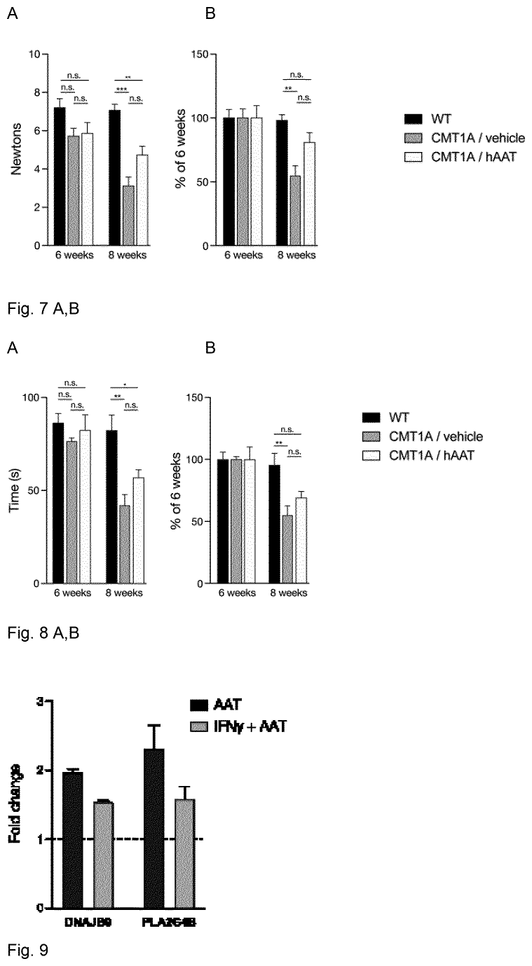

Grip strength test measures neuromuscular strength by assessing the animal's grasp of a metal grid. Lower grip strength was observed in the CMT groups compared to the wild type control group at the baseline (6 weeks old). At the baseline, the differences of grip strengths between groups were not statistically significant. As expected, a strong and significant decrease of grip strength was observed in the CMT1A+vehicle group compared to the wild type control group at 8 weeks old. Results show improvement of the grip strength for CMT1A mice treated with AAT compared to the control group (FIG. 7).

TABLE 16
Grip strength
Grip strenght at 6 weeks old (baseline) (Newtons)

| | | | | |
|---|---|---|---|---|
| Animal number | set 1 | set 2 | set 3 | Mean/animal |

WT control

| | | | | |
|---|---|---|---|---|
| 1.1 | 5.45 | 6.96 | 8.64 | 7.02 |
| 1.2 | 3.51 | 6.73 | 9.20 | 6.48 |
| 1.3 | 8.84 | 6.86 | 8.61 | 8.10 |

CMT1A + vehicle

| | | | | |
|---|---|---|---|---|
| 2.1 | 1.71 | 6.57 | 7.76 | 5.35 |
| 2.2 | 4.74 | 5.76 | 5.29 | 5.26 |
| 2.3 | 6.90 | 5.09 | 7.59 | 6.53 |

CMT1A + AAT

| | | | | |
|---|---|---|---|---|
| 3.1 | 5.84 | 7.62 | 1.82 | 5.10 |
| 3.2 | 7.10 | 7.19 | 6.62 | 6.97 |
| 3.3 | 5.94 | 3.08 | 7.52 | 5.51 |

Grip strenght at 8 weeks old (Newtons)

| | | | | |
|---|---|---|---|---|
| Animal number | set 1 | set 2 | set 3 | Mean/animal |

WT control

| | | | | |
|---|---|---|---|---|
| 1.1 | 6.31 | 4.90 | 8.83 | 6.68 |
| 1.2 | 5.46 | 7.51 | 7.41 | 6.79 |
| 1.3 | 6.46 | 9.38 | 7.29 | 7.71 |

CMT1A + vehicle

| | | | | |
|---|---|---|---|---|
| 2.1 | 4.29 | 4.76 | 2.90 | 3.98 |
| 2.2 | 5.58 | 1.27 | 2.05 | 2.96 |
| 2.3 | 0.61 | 3.43 | 3.16 | 2.40 |

CMT1A + AAT

| | | | | |
|---|---|---|---|---|
| 3.3 | 6.55 | 4.38 | 5.43 | 5.45 |
| 3.2 | 4.01 | 6.93 | 3.63 | 4.86 |
| 3.3 | 2.76 | 3.67 | 5.21 | 3.88 |

TABLE 17
mean grip strength
Grip strength, newtons (Mean ± SEM)

| | 6 weeks old | 8 weeks old |
|---|---|---|
| WT control | 7.20 ± 0.48 | 7.06 ± 0.33 |
| CMT1A + vehicle | 5.71 ± 0.41 | 3.12 ± 0.46*** |
| CMT1A + AAT | 5.86 ± 0.57 | 4.73 ± 0.46** |

2-way ANOVA with repeated measures and Bonferroni t-test
**; ***p < 0.01; p < 0.001 vs WT control

The rotarod test measures neuromuscular coordination by assessing the capacity of the animals to stay in balance on a rotating cylinder. Similar rotarod latency was observed between groups at the baseline (6 weeks old). As expected, a strong and significant decrease of rotarod latency was observed in the CMT1A+vehicle group compared to the wild type control group at 8 weeks old. Results show improvement of the rotarod latency for CMT1A mice treated with AAT compared to the control group (FIG. 8).

TABLE 18
rotarod latency
Rotarod latency at 6 weeks old (baseline) (seconds)

| | | | | |
|---|---|---|---|---|
| Animal number | set 1 | set 2 | set 3 | Mean/animal |

WT control

| | | | | |
|---|---|---|---|---|
| 1.1 | 64.7 | 94.3 | 88.4 | 82.46 |
| 1.2 | 105.8 | 98.8 | 84.9 | 96.49 |
| 1.3 | 77.2 | 115.0 | 46.3 | 79.50 |

CMT1A + vehicle

| | | | | |
|---|---|---|---|---|
| 2.1 | 69.5 | 82.5 | 65.8 | 72.62 |
| 2.2 | 56.6 | 77.8 | 101.8 | 78.77 |
| 2.3 | 61.2 | 72.3 | 84.3 | 72.62 |

CMT1A + AAT

| | | | | |
|---|---|---|---|---|
| 3.1 | 85.0 | 102.6 | 65.5 | 84.36 |
| 3.2 | 51.4 | 93.4 | 68.8 | 71.19 |
| 3.3 | 59.6 | 92.1 | 67.1 | 72.91 |

Rotarod latency at 8 weeks old (seconds)

| | | | | |
|---|---|---|---|---|
| Animal number | set 1 | set 2 | set 3 | Mean/animal |

WT control

| | | | | |
|---|---|---|---|---|
| 1.1 | 61.9 | 106.2 | 84.0 | 84.03 |
| 1.2 | 60.0 | 72.9 | 67.4 | 66.77 |
| 1.3 | 110.8 | 70.2 | 106.2 | 95.69 |

CMT1A + vehicle

| | | | | |
|---|---|---|---|---|
| 2.1 | 30.6 | 78.2 | 51.0 | 53.26 |
| 2.2 | 40.8 | 51.0 | 26.3 | 39.36 |
| 2.3 | 35.7 | 38.3 | 24.9 | 32.95 |

CMT1A + AAT

| | | | | |
|---|---|---|---|---|
| 3.1 | 62.1 | 35.7 | 51.5 | 49.76 |
| 3.2 | 83.2 | 39.5 | 71.3 | 64.65 |
| 3.3 | 46.0 | 46.0 | 76.4 | 56.12 |

TABLE 19
mean rotarod latency
Rotarod latency, seconds (Mean ± SEM)

| | 6 weeks old | 8 weeks old |
|---|---|---|
| WT control | 86.15 ± 5.24 | 82.16 ± 8.40 |
| CMT1A + vehicle | 74.67 ± 2.05 | 41.86 ± 5.99*** |
| CMT1A + AAT | 76.15 ± 4.13 | 56.84 ± 4.31* |

2-way ANOVA with repeated measures and Bonferroni t-test
*; ***p < 0.05; p < 0.001 vs WT control

### Example 11

It appears that PMP22 protein is particularly important in protecting nerves from physical pressure, helping them restore their structure after being pinched or squeezed (compressed). Compression can interrupt nerve signaling, leading to the sensation commonly referred to as a limb “falling asleep.” The ability of nerves to recover from normal, day-to-day compression, for example when sitting for long periods, keeps the limbs from constantly losing sensation. In CMT1A patients the myelination process is not properly complete, and the pathological symptoms associated with the disease become apparent most often after the second decade of life.

The PMP22 gene also plays a role in the growth of Schwann cells and the process by which cells mature to carry out specific functions (differentiation). Before they become part of myelin, newly produced PMP22 proteins are processed and packaged in specialized cell structures called the endoplasmic reticulum and the Golgi apparatus. Completion of these processing and packaging steps is critical for proper myelin function. CMT1A's pathomechanics is characterized by the absence of myelin sheaths due to an extra PMP22 gene, which is responsible for the abnormally high concentration of the peripheral myelin protein 22 (PMP22) in Schwann cells. GSEA analysis done on human microglial cells, AAT treatment has shown upregulation of genes related to the unfolded protein response (UPR) pathway and cell-survival (anti-apoptotic (FIG. 4C, FIG. 9).

### Example 12

ADAM17, also known as TACE, is a transmembrane protein that includes an extracellular zinc-dependent protease domain. In the context of CMT1A, ADAM17 is known for its inhibitory effect on SCs mediated myelination through neuregulin 1 type III (NRG1-III). It is postulated that AAT was able to cross the blood nerve barrier (BNB) and interact with ADAM17 to successfully inhibit its activity and by doing so allowing SCs to “manually” overcome the distress signal that an overloaded ER with PMP22 generates and facilitating the formation of myelin sheaths around axons.

### Example 13 Plasma hAAT Levels

hAAT was not detected in plasma of the wild type control and CMT1A mice treated with vehicle at the analyzed time points (day 14, day 19, day 24 and day 29). The hAAT was detected in plasma of CMT1A+hAAT group at a mean of 6.07 μg/mL, 6.99 μg/mL, 8.14 μg/mL and 5.22 μg/mL at day 14, day 19, day 24 and day 29 respectively.

### Example 14 Sciatic Nerve Histology

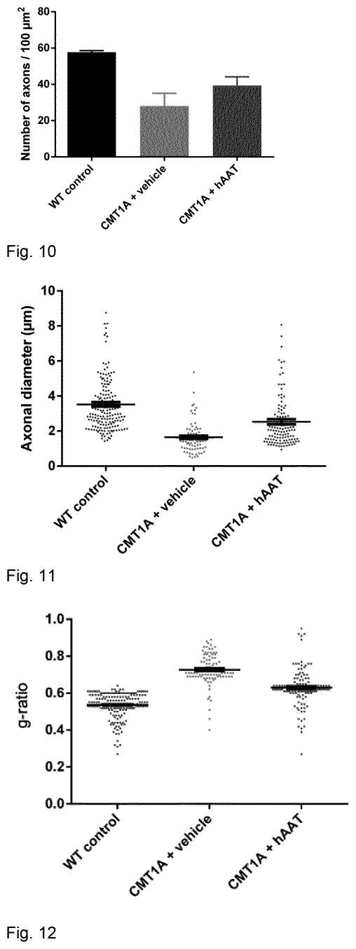

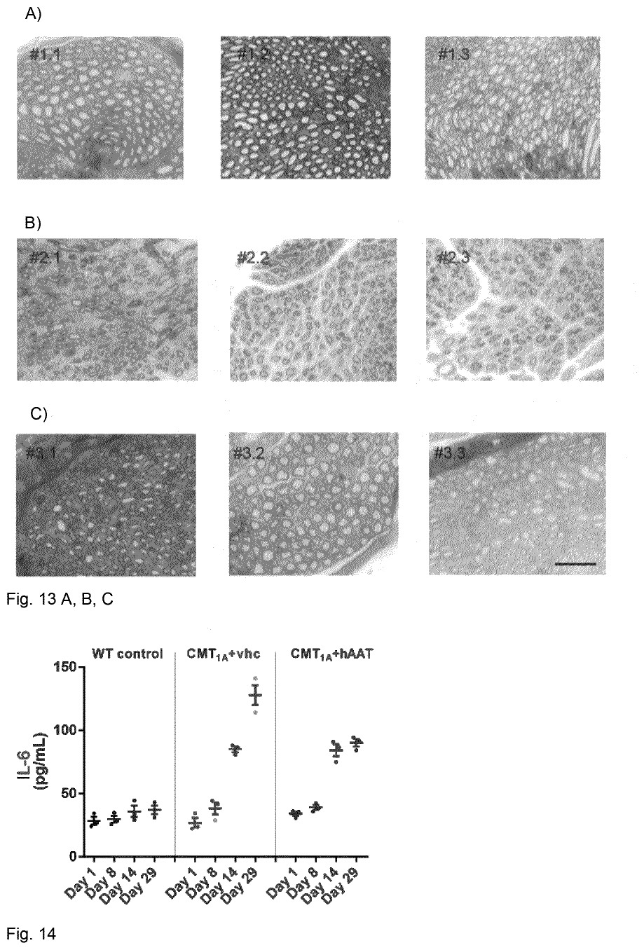

As expected a decrease of the total number of axons per surface, the axonal diameter and a significant increase of the g-ratio was observed in the CMT1A+vehicle group compared to the wild type control group at 8 weeks old (Table 20, FIG. 10 to FIG. 13).

Slight increase of the total number of axons per surface was observed in the CMT1A+hAAT treated group compared to the vehicle group. Moreover, significant increase of the axonal diameter and decrease of the g-ratio (equal to the ratio of the inner-to-outer diameter of a myelinated axon) were observed in the CMT1A animals treated with hAAT compared to the vehicle treated group. Even if, the CMT1A+hAAT animals also presented a number of axons, axonal diameter and g-ration statistically different than the wild type control group (Table 20, FIGS. 10 to 13). Taken together these data suggest a positive but partial efficacy of hAAT on the histopathology induced by the CMT1A disorder when administrated at 50 mg/kg twice daily by subcutaneous route.

TABLE 20
Sciatic nerve histology
Sciatic nerve histology (Mean ± SEM)

| | Axons/100 μm² | Axonal diameter (μm) | g-ratio |
|---|---|---|---|
| WT control | 57.67 ± 0.88 | 3.52 ± 0.13 | 0.54 ± 0.005 |
| CMT1A + vehicle | 28.00 ± 7.02* | 1.65 ± 0.09*** | 0.73 ± 0.010*** |
| CMT1A + AAT | 39.33 ± 4.81 | 2.53 ± 0.15***^{†††} | 0.63 ± 0.009***^{†††} |

1-way ANOVA and Tukey test
***p < 0.001 vs WT control;
^{†††}p < 0.001 CMT1A + vehicle

### Example 14—IL-6

Similar plasma IL-6 concentrations were observed between groups at the baseline (day 1) and day 8.

As expected, significant increase of plasma IL-6 concentration was observed in the CMT+vehicle group at day 14 and day 29.

The CMT+hAAT treated group also presented a significant increase of plasma IL-6 concentration compared to the baseline concentration. However, the plasma IL-6 concentration of animals treated with hAAT was lower than animals treated with vehicle at day 29 (Table 21 and FIG. 14) suggesting a direct or indirect effect of hAAT on this inflammatory cytokine.

TABLE 21
Plasma IL-6 levels
Plasma IL-6 concentration, pg/mL (Mean ± SD)

| | Day 1 | Day 8 | Day 14 | Day 29 |
|---|---|---|---|---|
| WT control | 28.50 ± 3.04 | 29.74 ± 2.67 | 35.90 ± 4.516 | 37.20 ± 3.391 |
| CMT1A + vehicle | 26.90 ± 3.86 | 38.32 ± 4.78 | 85.04 ± 2.296*** | 127.84 ± 7.810*** |
| CMT1A + AAT | 34.19 ± 1.67 | 39.10 ± 1.91* | 84.21 ± 4.727** | 90.20 ± 3.085***^{†} |

Student t-test
*; **; ***p < 0.05; p < 0.01; p < 0.001 vs WT control at this timepoint;
^{†}p < 0.05 vs CMT1A + vehicle at this timepoint

### Example 15—TNFα

As expected, significant increase of plasma TNF-α concentration was observed in the CMT1A+vehicle group at day 14 and day 29.

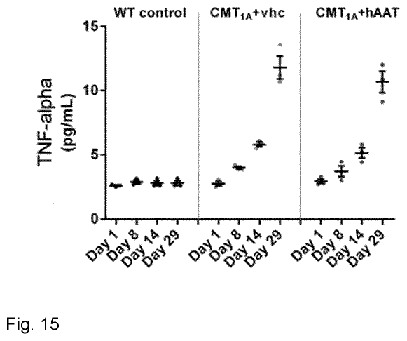

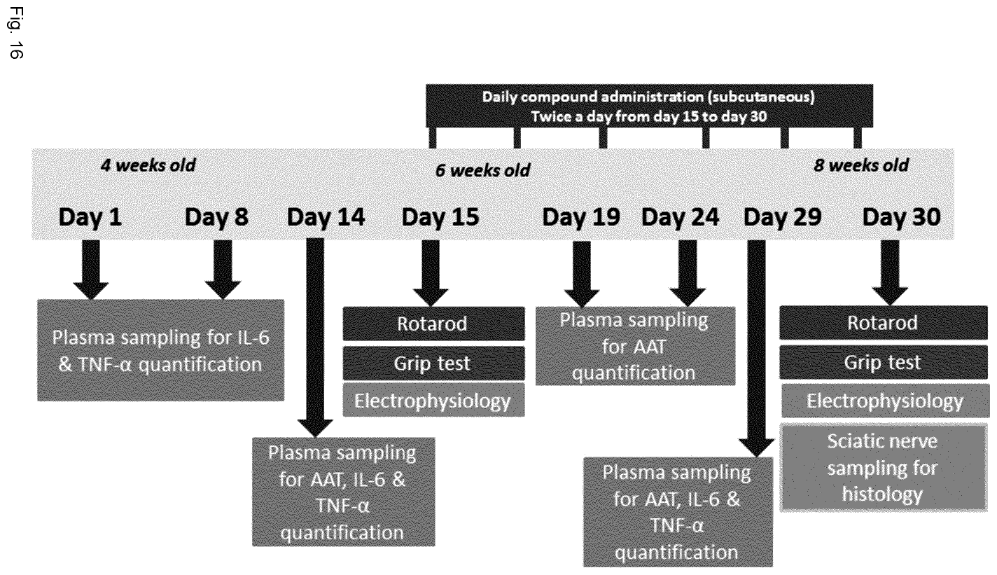

The CMT1A+hAAT treated group also presented a significant increase of plasma TNF-α concentration at day 14 and day 29 compared to the baseline concentration (Table 22 and FIG. 15) suggesting that hAAT has no effect on the levels of this inflammatory cytokine in this model of CMT1A.

TABLE 22
Plasma TNFα levels
Plasma TNFα concentration, pg/mL (Mean ± SEM)

| | Day 1 | Day 8 | Day 14 | Day 29 |
|---|---|---|---|---|
| WT control | 2.60 ± 0.05 | 2.89 ± 0.14 | 2.80 ± 0.187 | 2.80 ± 0.187 |
| CMT1A + vehicle | 2.76 ± 0.17 | 3.99 ± 0.11** | 5.80 ± 0.175*** | 11.81 ± 0.897*** |
| CMT1A + AAT | 2.94 ± 0.17 | 3.70 ± 0.41 | 5.15 ± 0.393** | 10.67 ± 0.833*** |

Student t-test
*; **; ***p < 0.05; p < 0.01; p < 0.001 vs WT control at this timepoint.

### Example 16

AAT's effect on SH-SY5Y treated with 6-OHDA was evaluated by cell morphology and cell proliferation followed by cell viability quantification.

### Cells and Treatments

SH-SY5Y cells, which are commonly used to model neurodegenerative disorders, were used to produce an in vitro Parkinson's model (Que R et al., Front. Immunol 2021). Cells were cultured in DMEMF12/Glutamax supplemented with 10% FBS.

Cells were treated for 24 hours with the neurotoxin 6-hydroxydopamine (6-OHDA, Sigma Aldrich 162957) at 50, 100 μM concentrations alone or in combination with AAT 25 μM (AAT plasma derived, Sigma Aldrich batch A9024). Cells treated with AAT alone or in combination with 60HDA for 24 hours, were then incubated with fresh AAT for additional 24 hours. Control cells were treated with PBS.

### Cell Growth and Viability Assay

At day 0 cells were plated in equally number in 24-well plate, the day after they were treated as described above, and final total cell count was performed at day 4 of culture (cell growth). Cell viability was assessed with Cell Counting Kit-8 (CCK-8; Sigma Aldrich 96992). After treatments, cells were incubated with 10 μl of CCK-8 solution for 2 hours in the incubator. Absorbance was measured at 450 nm using SpectraMax iD3 Microplate Reader. Experiments were performed in triplicate.

### Human IL-6 Immunoassay

Human 11-6 immunoassay (R&D D6050) was performed on cells supernatant. Briefly, 40000 cells were plated in 24-well plate and treated the day after as described in paragraph “Cells and treatment”. At the end of the treatment, cells were washed with PBS and incubated with 2% FBS medium for 24 hours, then cell culture supernatants were collected and centrifuged to remove particulates. Assay procedure was performed as described by manufactured instructions, on standard and samples duplicates. Absorbance was measured at 450 nm using SpectraMax iD3 Microplate Reader. A standard curve was prepared from seven IL-6 standard dilutions and IL-6 sample concentrations determined.

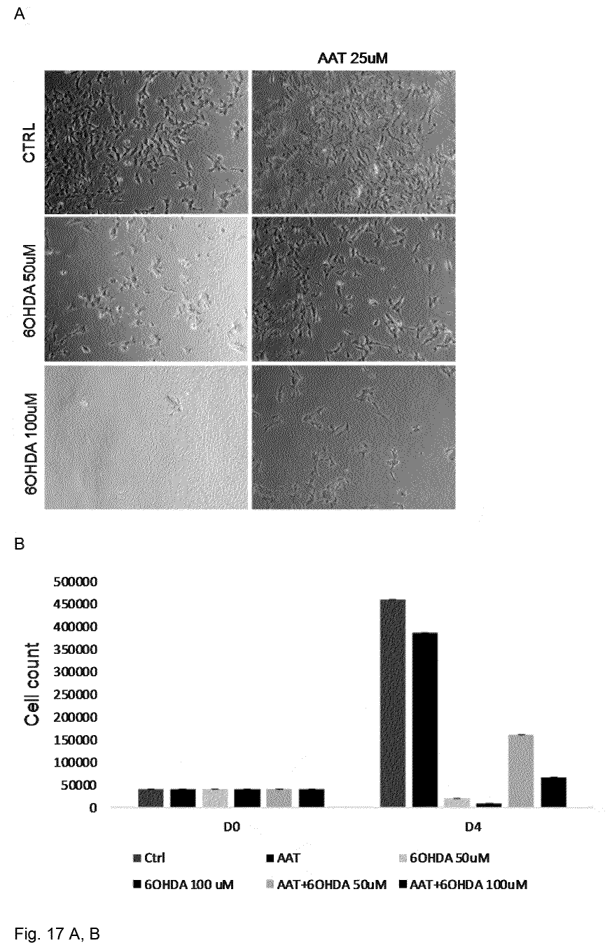

6-OHDA at 50-100 μM for 24 hours induced cells proliferation impairment by inducing cell death and reducing the number of cells after 4 days of culture (FIG. 17). In contrast combined treatment with AAT significantly increased the number of cells counted compared to 6-OHDA alone (FIG. 17), indicating a positive effect of AAT on cell survival/proliferation. T-test p-values are significative for: ctrl vs 6ohda p=0.001; 6ohda vs AAT+6ohda p=0.003. Ctrl vs AAT is not significant. Error bar are S.E.M.

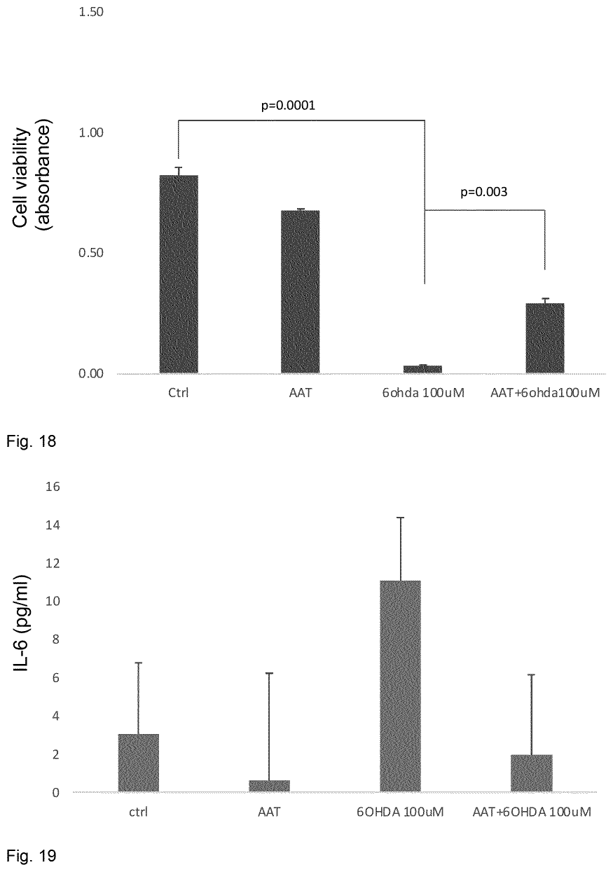

Treated cells were then challenged in a cell viability assay. SH-SY5Y treated with 6-OHDA alone showed strong reduction of cell viability compared to control cells and cells treated with AAT only (FIG. 18).

Cell count as well as the viability of the cells were significantly enhanced when 6-OHDA treatment was combined with AAT, compared to 6-OHDA treatment alone (FIG. 18). P-values are calculated with t-test, not significant differences were observed between control and AAT alone. All conditions were performed in triplicates. Based on these results we can conclude that AAT administration in a PD cell model (SH-SY5Y induced by 6-OHDA) has favorable effect on cell growth and viability, possibly protecting cells from 6-OHDA induced death.

In addition to explore the role of AAT on pro-inflammatory cytokines we performed IL-6 quantification in cells supernatant. Medium from cells induced by 6-OHDA had increased level of IL-6 compared to control medium, on the contrary the medium collected from cells treated with AAT displayed less concentration of IL-6 (FIG. 19). T-test p-value are significant for ctrl vs 6-OHDA p=0.05 and not significant for the other comparison.

### REFERENCES

1. Wrapp, D., et al., Cryo-EM structure of the 2019-nCoV spike in the prefusion conformation. Science, 2020. 367(6483): p. 1260-1263.
2. Jaimes, J. A., J. K. Millet, and G. R. Whittaker, Proteolytic Cleavage of the SARS-CoV-2 Spike Protein and the Role of the Novel S1/S2 Site. iScience, 2020. 23(6): p. 101212.
3. Korber, B., et al., Spike mutation pipeline reveals the emergence of a more transmissible form of SARS-CoV-2. bioRxiv, 2020: p. 2020.04.29.069054.
4. Becerra-Flores, M. and T. Cardozo, SARS-CoV-2 viral spike G614 mutation exhibits higher case fatality rate. Int J Clin Pract, 2020.
5. Li, Q., et al., The Impact of Mutations in SARS-CoV-2 Spike on Viral Infectivity and Antigenicity. Cell, 2020.
6. Ogawa, J., et al., The D614G mutation in the SARS-CoV-2 Spike protein increases infectivity in an ACE2 receptor dependent manner. bioRxiv, 2020.
7. Goodfellow, I. Goodfellow, and S. I. Taube, Calicivirus Replication and Reverse Genetics Viral Gastroenteritis: Molecular Epidemiology and Pathogenesis. 2016: p. 355-378.
8. Yin, W., et al., Structural basis for inhibition of the RNA-dependent RNA polymerase from SARS-CoV-2 by remdesivir. Science, 2020.
9. Gao, Y., et al., Structure of the RNA-dependent RNA polymerase from COVID-19 virus. Science, 2020. 368(6492): p. 779-782.
10. Pachetti, M., et al., Emerging SARS-CoV-2 mutation hot spots include a novel RNA-dependent-RNA polymerase variant. J Transl Med, 2020. 18(1): p. 179.
11. Baecher-Allan, C., B. J. Kaskow, and H. L. Weiner. 2018. ‘Multiple Sclerosis: Mechanisms and Immunotherapy’, Neuron, 97: 742-68.
12. Bergin, D. A., K. Hurley, N. G. McElvaney, and E. P. Reeves. 2012. ‘Alpha-1 antitrypsin: a potent anti-inflammatory and potential novel therapeutic agent’, Arch Immunol TherExp (Warsz), 60: 81-97.
13. Blauwendraat, C., M. A. Nails, and A. B. Singleton. 2020. ‘The genetic architecture of Parkinson's disease’, Lancet Neurol, 19: 170-78.
14. Chang, K. H., T. J. Chuang, R. K. Lyu, L. S. Ro, Y. R. Wu, H. S. Chang, C. C. Huang, H. C. Kuo, W. C. Hsu, C. C. Chu, and C. M. Chen. 2012. ‘Identification of gene networks and pathways associated with Guillain-Barre syndrome’, PLoS One, 7: e29506.
15. Dias Carvalho, P., C. F. Guimaraes, A. P. Cardoso, S. Mendonca, A. M. Costa, M. J. Oliveira, and S. Velho. 2018. ‘KRAS Oncogenic Signaling Extends beyond Cancer Cells to Orchestrate the Microenvironment’, Cancer Res, 78: 7-14.
16. Hampel, H., F. Caraci, A. C. Cuello, G. Caruso, R. Nistico, M. Corbo, F. Baldacci, N. Toschi, F. Garaci, P. A. Chiesa, S. R. Verdooner, L. Akman-Anderson, F. Hernandez, J. Avila, E. Emanuele, P. L. Valenzuela, A. Lucia, M. Watling, B. P. Imbimbo, A. Vergallo, and S. Lista. 2020. ‘A Path Toward Precision Medicine for Neuroinflammatory Mechanisms in Alzheimer's Disease’, Front Immunol, 11: 456.
17. Hoyle, J. C., M. C. Isfort, J. Roggenbuck, and W. D. Arnold. 2015. ‘The genetics of Charcot-Marie-Tooth disease: current trends and future implications for diagnosis and management’, Appl Clin Genet, 8: 235-43.
18. Marogianni, C., M. Sokratous, E. Dardiotis, G. M. Hadjigeorgiou, D. Bogdanos, and G. Xiromerisiou. 2020. ‘Neurodegeneration and Inflammation—An Interesting Interplay in Parkinson's Disease’, Int J Mol Sci, 21.
19. Martin-Aguilar, L., E. Pascual-Goni, and L. Querol. 2019. ‘Autoantibodies in immune-mediated inflammatory neuropathies’, Med Clin (Barc), 153: 360-67.
20. Matthews, P. M. 2019. ‘Chronic inflammation in multiple sclerosis—seeing what was always there’, Nat Rev Neurol, 15: 582-93.
21. Nikolac Perkovic, M., and N. Pivac. 2019. ‘Genetic Markers of Alzheimer's Disease’, Adv Exp Med Biol, 1192: 27-52.
22. Parnell, G. P., and D. R. Booth. 2017. ‘The Multiple Sclerosis (MS) Genetic Risk Factors Indicate both Acquired and Innate Immune Cell Subsets Contribute to MS Pathogenesis and Identify Novel Therapeutic Opportunities’, Front Immunol, 8: 425.
23. Ransohoff, R. M. 2016. ‘A polarizing question: do M1 and M2 microglia exist?’, Nat Neurosci, 19: 987-91.
24. Satoh, J. I. 2017. ‘Gene Expression Profiles of M1 and M2 Microglia Characterized by Comparative Analysis of Public Datasets’, Clinical and Experimental Neuroimmunology, 9(2): 124-138.
25. Skaper, S. D., L. Facci, M. Zusso, and P. Giusti. 2018. ‘An Inflammation-Centric View of Neurological Disease: Beyond the Neuron’, Front Cell Neurosci, 12: 72.
26. Timmerman, V., A. V. Strickland, and S. Zuchner. 2014. ‘Genetics of Charcot-Marie-Tooth (CMT) Disease within the Frame of the Human Genome Project Success’, Genes (Basel), 5: 13-32.
27. Zhou, T., Z. Huang, X. Zhu, X. Sun, Y. Liu, B. Cheng, M. Li, Y. Liu, C. He, and X. Liu. 2018. ‘Alpha-1 Antitrypsin Attenuates M1 Microglia-Mediated Neuroinflammation in Retinal Degeneration’, Front Immunol, 9: 1202.
28. Que R, Zheng J, Chang Z, Zhang W, Li H, Xie Z, Huang Z, Wang H-T, Xu J, Jin D, Yang W, Tan E-Kand Wang Q DI-3-n-Butylphthalide Rescues Dopaminergic Neurons in Parkinson's Disease Models by Inhibiting the NLRP3 In “ammasome and Ameliorating Mitochondrial Impairment. Front. Immunol. 12 (2021).

1. A method for treating a disease or disorder of the nervous system in a patient, the method comprising administering to the patient a composition comprising a therapeutically effective amount of an alpha1-antitrypsin (AAT) protein, a variant, an isoform and/or a fragment thereof.

2. A method of treating a disease or disorder of the nervous system in a patient, the method comprising administering to the patient a vector comprising a nucleic acid sequence encoding an AAT protein.

3. A method of treating a disease or disorder of the nervous system in a patient, the method comprising administering to the patient a genetically modified cell comprising a nucleic acid sequence encoding an AAT protein.

4. The method of claim 1, wherein the disease or disorder of the nervous system is a disease or disorder of the peripheral nervous system.

5. The method of claim 4, wherein the disease or disorder of the peripheral nervous system is motor and sensory neuropathy of the peripheral nervous system.

6. The method of claim 5, wherein the sensory neuropathy of the peripheral nervous system is a hereditary motor and sensory neuropathy of the peripheral nervous system.

7. The method of claim 6, wherein the hereditary motor and sensory neuropathy of the peripheral nervous system is Charcot-Marie-Tooth disease or a symptom thereof, preferably at least one symptom selected from the group consisting of weakness in legs, ankles and/or feet, loss of muscle bulk in legs and/or feet, high foot arches, curled toes, decreased ability to run, difficulty lifting foot at the ankle, abnormal gait, frequent tripping or falling and decreased sensation or a loss of feeling in legs and/or feet.

8. The method of claim 1, wherein the disease or disorder of the nervous system is an inflammatory disease or disorder of the nervous system.

9. The method of claim 8, wherein the inflammatory disease or disorder of the nervous system is a myeloid cell-mediated disease or disorder of the nervous system.

10. The method of claim 1, wherein the disease or syndrome of the nervous system is a disease or syndrome selected from the group of Parkinson's disease, dementia, multiple sclerosis, amyotrophic lateral sclerosis, Alzheimer's disease, and Huntington's disease.

11. The method of claim 1, wherein the disease or disorder of the nervous system is at least one symptom of a disease or disorder of the nervous system selected from the group consisting of: tremor, memory loss, slurred speech, dizziness, change in vision and headache.

12. The method of claim 1, wherein the AAT protein, a variant, an isoform and/or a fragment thereof is human plasma-extracted.

13. The method of claim 1, wherein the alpha1-antitrypsin (AAT) protein, a variant, an isoform and/or a fragment thereof is recombinant alpha1-antitrypsin (rhAAT), a variant, an isoform and/or a fragment thereof.

14. The method of claim 1, wherein the composition comprises at least one pharmaceutical carrier.

15. The method of claim 1, wherein the pharmaceutical carrier is a blood-brain barrier permeability enhancer.

16. The method of claim 1, wherein the composition is formulated for intracerebral administration, intravenous injection, intravenous infusion, infusion with a dosator pump, inhalation nasal-spray, eye-drops, skin-patches, slow release formulations, ex vivo gene therapy or ex vivo cell-therapy.


SEQUENCE LISTING (8 sequences)

SEQ ID NO: 1 - protein, 418 aa, Homo sapiens
  AAT
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 44 aa, Artificial Sequence
  C-terminal AAT sequence 374-418
MFLEAIPMSIPPEVKFNKPFVFLMIEQNTKSPLFMGKVVNPTQK

SEQ ID NO: 3 - protein, 11 aa, Artificial Sequence
  Fluorogenic peptides derived from SARS-CoV-2 spike
HTVSLLRSTSQ

SEQ ID NO: 4 - protein, 11 aa, Artificial Sequence
  Fluorogenic peptides derived from SARS-CoV-2 spike
TNSPRRARSVA

SEQ ID NO: 5 - protein, 10 aa, Artificial Sequence
  HIV-TAT 48-57 peptide
GRKKRRQRRR

SEQ ID NO: 6 - protein, 15 aa, Artificial Sequence
  FHV-coat 35-49 peptide
RRRRNRTRRNRRRVR

SEQ ID NO: 7 - protein, 13 aa, Artificial Sequence
  HTLV-II Rex 4-16 peptide
TRRQRTRRARRNR

SEQ ID NO: 8 - protein, 19 aa, Artificial Sequence
  BMV gag 7-25 peptide
KMTRAQRRAAARRNRWTAR